(19) United States
(12) Patent Application Publication
(10) Pub. No.: US 2015/0165243 A1
(43) Pub. Date: Jun. 18, 2015

(54) System and Method for Treating Cartilage and Injuries to Joints and Connective Tissue

(71) Applicant: Guided Therapy Systems, LLC, Mesa, AZ (US)

(72) Inventors: Michael H. Slayton, Tempe, AZ (US); Peter G. Barthe, Phoenix, AZ (US)

(73) Assignee: GUIDED THERAPY SYSTEMS, LLC, Mesa, AZ (US)

(21) Appl. No.: 14/628,198

(22) Filed: Feb. 20, 2015

Related U.S. Application Data
(63) Continuation-in-part of application No. 13/136,542, filed on Aug. 2, 2011.
Application No. 13/136,542 is a continuation-in-part of application No. 13/545,945, filed on Jul. 10, 2012.
Application No. 13/545,945 is a continuation-in-part of application No. 13/136,538, filed on Aug. 2, 2011.
Application No. 13/136,538 is a continuation-in-part of application No. 13/965,741, filed on Aug. 13, 2013.
Application No. 13/965,741 is a continuation of application No. 13/835,635, filed on Mar. 15, 2013, now Pat. No. 8,915,853.
Application No. 13/835,635 is a continuation of application No. 13/494,856, filed on Jun. 12, 2012, now Pat. No. 8,444,562.
Application No. 13/494,856 is a continuation-in-part of application No. 11/857,989, filed on Sep. 19, 2007, now abandoned.
Application No. 13/494,856 is a continuation-in-part of application No. 12/028,636, filed on Feb. 8, 2008, now Pat. No. 8,535,228.
Application No. 12/028,636 is a continuation-in-part of application No. 11/163,151, filed on Oct. 6, 2005, now abandoned.
Application No. 12/028,636 is a continuation-in-part of application No. 11/163,148, filed on Oct. 6, 2005, now abandoned.
Application No. 13/965,741 is a continuation-in-part of application No. 12/437,726, filed on May 8, 2009.
Application No. 12/437,726 is a continuation of application No. 10/950,112, filed on Sep. 24, 2004, now Pat. No. 7,530,958.
(60) Provisional application No. 61/369,782, filed on Aug. 2, 2010.
(60) Provisional application No. 61/369,793, filed on Aug. 2, 2010.
(60) Provisional application No. 61/369,806, filed on Aug. 2, 2010.
(60) Provisional application No. 61/370,095, filed on Aug. 2, 2010.
(60) Provisional application No. 61/506,125, filed on Jul. 10, 2011.
(60) Provisional application No. 61/506,127, filed on Jul. 10, 2011.
(60) Provisional application No. 61/506,126, filed on Jul. 10, 2011.
(60) Provisional application No. 61/506,160, filed on Jul. 10, 2011.
(60) Provisional application No. 61/506,163, filed on Jul. 10, 2011.
(60) Provisional application No. 61/369,782, filed on Aug. 2, 2010.
(60) Provisional application No. 61/369,793, filed on Aug. 2, 2010.
(60) Provisional application No. 61/369,806, filed on Aug. 2, 2010.
(60) Provisional application No. 61/370,095, filed on Aug. 2, 2010.
(60) Provisional application No. 60/826,199, filed on Sep. 19, 2006.
(60) Provisional application No. 60/616,755, filed on Oct. 6, 2004.
(60) Provisional application No. 60/616,754, filed on Oct. 6, 2004.

(51) Int. Cl. A61N 7/02; A61N 7/00; A61M 37/00

(52) U.S. Cl. CPC A61N 7/02; A61M 37/0092; A61N 7/00; A61N 2007/0013

(57) ABSTRACT

Various embodiments provide systems and methods of treating damaged cartilage injuries to joints and connective tissue. Methods and systems useful for permanent relief of pain in joints are also provided herein. Various embodiments provide for combining therapeutic ultrasound energy directed to a joint with a medicant injected into the joint.

### CROSS-REFERENCE TO RELATED APPLICATIONS

This application is a continuation-in-part and claims the benefit of co-pending U.S. patent application Ser. No. 13/136,542 filed on Aug. 2, 2011, which claims the benefit of U.S. Provisional Patent Application Ser. No. 61/369,782, entitled “Systems and Methods for Ultrasound Treatment”, filed Aug. 2, 2010; U.S. Provisional Patent Application Ser. No. 61/369,793, entitled “System and Method for Treating Sports Related Injuries”, filed Aug. 2, 2010; U.S. Provisional Patent Application Ser. No. 61/369,806, entitled “System and Method for Treating Sports Related Injuries”, filed Aug. 2, 2010; U.S. Provisional Patent Application Ser. No. 61/370,095, entitled “System and Method for Treating Cartilage”, filed Aug. 2, 2010; all of which are incorporated by reference herein.

This continuation-in-part also claims benefit of co-pending U.S. patent application Ser. No. 13/545,945 which is a continuation-in-part of U.S. patent application Ser. No. 13/136,538 entitled “Systems and Methods for Treating Acute and/or Chronic Injuries in Soft Tissue,” filed Aug. 2, 2011, which claims priority to and the benefit of U.S. Provisional Patent Application Ser. No. 61/369,782, entitled “Systems and Methods for Ultrasound Treatment”, filed Aug. 2, 2010; U.S. Provisional Patent Application Ser. No. 61/369,793, entitled “System and Method for Treating Sports Related Injuries”, filed Aug. 2, 2010; U.S. Provisional Patent Application Ser. No. 61/369,806, entitled “System and Method for Treating Sports Related Injuries”, filed Aug. 2, 2010; U.S. Provisional Patent Application Ser. No. 61/370,095, entitled “System and Method for Treating Cartilage”, filed Aug. 2, 2010; all of which are incorporated by reference herein.

This continuation-in-part claims priority to and the benefit of U.S. Provisional Patent Application Ser. No. 61/506,125, entitled “Systems and Methods for Creating Shaped Lesions” filed Jul. 10, 2011; U.S. Provisional Patent Application Ser. No. 61/506,127, entitled “Systems and Methods for Treating Injuries to Joints and Connective Tissue,” filed Jul. 10, 2011; U.S. Provisional Patent Application Ser. No. 61/506,126, entitled “System and Methods for Accelerating Healing of Implanted Materials and/or Native Tissue,” filed Jul. 10, 2011; US Provisional Patent Application Ser. No, 61/506,160, entitled “Systems and Methods for Cosmetic Rejuvenation,” filed Jul. 10, 2011; U.S. Provisional Patent Application Ser. No. 61/506,163, entitled “Methods and Systems for Ultrasound Treatment,” filed Jul. 10, 2011; all of which are incorporated by reference herein.

This continuation-in-part also claims priority to co-pending U.S. patent application Ser. No. 13/965,741 filed Aug. 13, 2013 which is a continuation of Ser. No. 13/835,635 filed Mar. 15, 2013 (now U.S. Pat. No. 8,915,853), which is a continuation of U.S. application Ser. No. 13/494,856 filed Jun. 12, 2012, now patented as U.S. Pat. No. 8,444,562, which is a continuation-in-part of U.S. application Ser. No. 11/857,989 filed Sep. 19, 2007, now abandoned, which claims the benefit of priority from U.S. Provisional No. 60/826,199 filed Sep. 19, 2006, each of which are incorporated in its entirety by reference, herein. U.S. application Ser. No. 13/494,856 is also a continuation-in-part of U.S. application Ser. No. 12/028,636 filed Feb. 8, 2008, which is a continuation-in-part of U.S. application Ser. No. 11/163,151 filed on Oct. 6, 2005, now abandoned, which in turn claims priority to U.S. Provisional Application No. 60/616,755 filed on Oct. 6, 2004, each of which are incorporated in its entirety by reference, herein. Further, U.S. application Ser. No. 12/028,636 is a continuation-in-part of U.S. application Ser. No. 11/163,148 filed on Oct. 6, 2005, now abandoned, which in turn claims priority to U.S. Provisional Application No. 60/616,754 filed on Oct. 6, 2004, each of which are incorporated in its entirety by reference, herein. U.S. application Ser. No. 13/494,856 is also a continuation-in-part of U.S. application Ser. No. 12/437,726 filed May 8, 2009, which is a continuation of U.S. application Ser. No. 10/950,112 filed Sep. 24, 2004 now patented as U.S. Pat. No. 7,530,958.

### STATEMENT REGARDING FEDERALLY SPONSORED RESEARCH

N/A

### BACKGROUND OF THE INVENTION

The matrix of cartilage is comprised of collagens, proteoglycans, and noncollagenous proteins and serves as the cushion and shock absorber within the joint as it lines the ends of the two bones that form the joint. For example, cartilage damage can be caused by several conditions including: joint injury, avascular necrosis, osteoarthritis, and rheumatoid arthritis. This damaged cartilage causes pain and can limit the motion of the joint. In order to fix the damaged cartilage, surgeon will have to cut into the joint to gain access to the damaged cartilage. What is needed, are new approaches to treating cartilage that employ non-invasive techniques.

Subcutaneous tissues such as, muscles, tendons, ligaments and cartilage, are important connective tissues that provide force and motion, non-voluntary motion, anchoring, stability, and support among other functions. These tissues are prone to wear and injury due to participation in sports or other daily activities which put stress on these tissues.

Inflammation is a response of a tissue to injury and is characterized by increased blood flow to the tissue causing increased temperature, redness, swelling, and pain. Inflammation can be classified as either acute or chronic. Acme inflammation is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process.

What is needed, are new approaches to treating injuries to joints. In addition, new approaches to managing pain are needed.

### SUMMARY OF THE INVENTION

Accordingly, methods of treating a damaged cartilage are provided. Such a method can include targeting the damaged cartilage in region of interest, directing therapeutic ultrasound energy to the damaged cartilage, ablating at least a portion of the damaged cartilage and improving the damaged cartilage. The method can include focusing therapeutic ultrasound energy to create at least one lesion in a portion of the damaged cartilage. The method can also include imaging the damaged cartilage. The method can include increasing blood perfusion to the region of interest. The method can include welding together the damaged cartilage with therapeutic ultrasound energy. The method can include cutting the damaged cartilage and removing it from the joint with therapeutic ultrasound energy. The method can include smoothing the cartilage with therapeutic ultrasound energy. The method can include regenerating cartilage. In one embodiment, the damaged cartilage is torn cartilage. Various embodiments provide a system for treating an injury to cartilage in a joint. In some embodiments, the system can include an arthroscopic probe having a housing on a distal end of the probe and a controller controlling the probe. The housing can contain an ultrasound transducer, a position sensor, a communication interface and a rechargeable power supply.

In some embodiments, the ultrasound transducer can be configured to focus a conformal distribution of ultrasound energy to ablate and fracture at least one of cartilage and surrounding tissue in an injury location. In some embodiments, the position sensor can be configured to communicate a position of the housing and a speed of movement of the housing. In some embodiments, the communication interface can be configured for wireless communication and communicates with the ultrasound transducer, and the position sensor. In some embodiments, the rechargeable power supply can supply power to the ultrasound transducer, the position sensor, and the communication interface.

Various embodiments provide a method of non-invasive micro-fraction surgery. The method can include the steps of identifying an injury location comprising cartilage; directing a conformal distribution of ultrasound energy to at least one of cartilage and surrounding subcutaneous tissue in the injury location; ablating the at least one of cartilage and surrounding subcutaneous tissue in the injury location; fracturing a portion of the cartilage in the injury location; initiating regrowth of the cartilage at the injury location; and sparing intervening tissue between a surface of skin above the injury location and the at least one of cartilage and surrounding subcutaneous tissue in the injury location.

In some embodiments, the method can include the step of welding a portion of the cartilage at the injury location with the conformal distribution of ultrasound energy. In some embodiments, the method can include the step of creating a plurality of micro ablations in at least one of the cartilage and the surrounding subcutaneous tissue in the injury location. In some embodiments, the method can include the step of increasing blood perfusion to the injury location. In some embodiments, the surrounding subcutaneous tissue is bone. In some embodiments, the method includes the step of fracturing a portion of the bone with the conformal distribution of ultrasound energy to initiate re-growth of the cartilage onto the bone.

Various embodiments described herein provide methods and systems for ultrasound treatment of tissue are provided, Accordingly, tissue such as muscle, tendon, ligament and/or cartilage, are treated with ultrasound energy. The ultrasound energy can be focused, unfocused or defocused and can be applied to a region of interest containing a joint to achieve a therapeutic effect.

Various embodiments described herein, provide a method for treating an injury in a joint of a body. In some embodiments the method comprises targeting a region of interest comprising the injury in the joint and tissue surrounding the joint and imaging the injury in the region of interest In addition, the method can comprise delivering ultrasound energy to the joint, creating a conformal region of elevated temperature in the joint, and initiating at least one thermally induced biological effect in the joint.

Various embodiments provide methods of treating an injury in a joint. In some embodiments, the method can comprise targeting injured fibrous son tissue located in at least one of at and proximate to an injury location comprising a portion of a joint and directing therapeutic ultrasound energy to the injured fibrous soft tissue. In some embodiments, the method can comprise creating a conformal region of elevated temperature in the injured fibrous soft tissue, and creating at least one thermally induced biological effect in the injured fibrous soft tissue.

Various embodiments provide a method of providing pain relief in a joint. In some embodiments, the method can comprise identifying a location of pain in a joint; imaging the location in the joint; and identifying a nerve ending responsible for the pain in the joint. In some embodiments, the method can further comprise focusing ultrasound energy onto the nerve ending responsible for the pain in the joint; ablating the nerve ending with the ultrasound energy; disabling function of the nerve ending; and eliminating the pain in the joint.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 illustrates a method of treatment, according to various embodiments.

FIG. 2 illustrates a cross sectional view of tissue layers and ultrasound energy directed to a muscle and connective tissue layer, according to various embodiments.

FIG. 3 illustrates a cross sectional view of tissue layers and ultrasound energy directed to at least one of cartilage and ligament tissues, according to various embodiments.

FIG. 4 illustrates various shapes of lesions, according to various embodiments.

FIG. 5 illustrates a treatment system, according to various embodiments.

FIG. 6 illustrates a treatment system comprising a position sensor, according to various embodiments.

FIG. 7 illustrates a ultrasound probe comprising a transducer and a motion mechanism, according to various embodiments.

FIG. 8 illustrates a ultrasound probe comprising a transducer, according to various embodiments.

FIG. 9 illustrates a hand held ultrasound probe, according to various embodiments.

FIG. 10 illustrates a plurality of exemplary transducer configurations, according to various embodiments.

FIG. 11 illustrates methods of treating a meniscus tear, according to various embodiments.

FIG. 12 illustrates methods of treating damaged cartilage, according to various embodiments.

FIG. 13 is a flow chart illustrating various methods, according to various non-limiting embodiments.

FIG. 14 is a cross sectional view illustrating ultrasound energy directed to a muscle and connective tissue layer, according to various non-limiting embodiments.

FIG. 15 is a cross sectional view illustrating ultrasound energy directed to at least one of muscle and tendon tissues, according to various non-limiting embodiments.

FIG. 16 is a cross sectional view illustrating ultrasound energy directed to at least one of cartilage and ligament tissues, according to various non-limiting embodiments.

FIG. 17 is a cross sectional view illustrating ultrasound energy directed to a joint, according to various non-limiting embodiments.

FIG. 18A illustrates an initial step of a method, according to various non-limiting embodiments.

FIG. 18B illustrates a subsequent step of a method, according to various non-limiting embodiments.

FIG. 18C illustrates a subsequent step of a method, according to various non-limiting embodiments.

FIG. 19A illustrates an initial step of a method, according to various non-limiting embodiments.

FIG. 19B illustrates a subsequent step of a method, according to various non-limiting embodiments.

FIG. 20A illustrates an initial step of a method, according to various embodiments.

FIG. 20B illustrates a subsequent step of a method, according to various non-limiting embodiments.

FIG. 20C illustrates a subsequent step of a method, according to various non-limiting embodiments.

FIG. 20D illustrates a subsequent step of a method, according to various non-limiting embodiments.

FIG. 21 is a flow chart illustrating method, according to various embodiments; and

FIG. 22 A illustrates an initial step of a method, according to various embodiments.

FIG. 22B illustrates a subsequent step of a method, according to various non-limiting embodiments.

FIG. 22C illustrates a subsequent step of a method, according to various non-limiting embodiments.

FIG. 22D illustrates a subsequent step of a method, according to various non-limiting embodiments.

### DETAILED DESCRIPTION OF THE INVENTION

The following description is merely exemplary in nature and is in no way intended to limit the various embodiments, their application, or uses. As used herein, the phrase “at least one of A, B, and C” should be construed to mean a logical (A or B or C), using a non-exclusive logical or. As used herein, the phrase “A, B and/or C” should be construed to mean (A, B, and C) or alternatively (A or B or C), using a non-exclusive logical or. It should be understood that steps within a method may be executed in different order without altering the principles of the present disclosure.

The drawings described herein are for illustrative purposes only of selected embodiments and not all possible implementations, and are not intended to limit the scope of any of the various embodiments disclosed herein or any equivalents thereof. It is understood that the drawings are not drawn to scale. For purposes of clarity, the same reference numbers will be used in the drawings to identify similar elements. The various embodiments may be described herein in terms of various functional components and processing steps. It should be appreciated that such components and steps may be realized by any number of hardware components configured to perform the specified functions. For example, various embodiments may employ various medical treatment devices, visual imaging and display devices, input terminals and the like, which may carry out a variety of functions under the control of one or more control systems or other control devices. In addition, the embodiments may be practiced in any number of medical contexts and that the various embodiments relating to a method and system for acoustic tissue treatment as described herein are merely indicative of exemplary applications. for the invention. For example, the principles, features and methods discussed may be applied to any medical application.

According to various embodiments, methods and systems useful for treating cartilage are provided herein. The methods and systems provided herein can be noninvasive, for example, no cutting or injecting into the skin is required. Treating damaged or injured cartilage using the methods and systems provided herein minimize recover time and may in some cases eliminate downtime for recovery. Further treating damaged or injured cartilage using the methods and systems provided herein minimize discomfort to a patient having such a procedure.

Various embodiments, described herein, provide methods of treating injured cartilage. Such a method can include targeting the damaged cartilage in region of interest, directing therapeutic ultrasound energy to the damaged cartilage, ablating at least a portion of the damaged cartilage and improving the damaged cartilage. The method can include focusing therapeutic ultrasound energy to create at least one lesion in a portion of the damaged cartilage. The method can also include imaging the damaged cartilage. The method can include increasing blood perfusion to the region of interest. The method can include welding together the damaged cartilage with therapeutic ultrasound energy. The method can include cutting the damaged cartilage and removing it from the joint with therapeutic ultrasound energy. The method can include smoothing the cartilage with therapeutic ultrasound energy. The method can include regenerating cartilage.

In some embodiments, damaged cartilage can be from a joint injury, avascular necrosis, osteoarthritis, and rheumatoid arthritis. In one embodiment, the damaged cartilage can be torn cartilage. In one embodiment, the damaged cartilage can be a torn meniscus. In one embodiment, the damaged cartilage is a partial tear in cartilage. In some embodiments, the damaged cartilage is not in a joint, but rather in a nose, an ear, in a face, or any other such location in a body. In various embodiments, the damaged cartilage is in a joint.

Various embodiments provide a system for treating damaged cartilage in a joint. In some embodiments, the system can include an arthroscopic probe having a housing on a distal end of the probe, such as for example an endoscope, and a controller controlling the probe. The housing can contain an ultrasound transducer, a position sensor, a communication interface and a rechargeable power supply.

In some embodiments, the ultrasound transducer can be configured to focus a conformal distribution of ultrasound energy to ablate and fracture at least one of cartilage and surrounding tissue in an injury location. In some embodiments, the position sensor. can be configured to communicate a position of the housing and a speed of movement of the housing. In some embodiments, the communication interface can be configured for wireless communication and communicates with the ultrasound transducer, and the position sensor. In some embodiments, the rechargeable power supply can supply power to the ultrasound transducer, the position sensor, and the communication interface.

In some embodiments, the controller communicates with the communication interface. In some embodiments, the controller can be configured to control a spatial parameter and a temporal parameter of the ultrasound transducer to emit the conformal distribution of ultrasound energy to ablate and fracture at least one of cartilage and surrounding tissue. In some embodiments, the controller can be configured to receive the position of the housing and the speed of movement of the housing, and can be configured to control the timing and location of conformal distribution of ultrasound energy based on the position and the speed.

In some embodiments, the ultrasound transducer can be a dual mode imaging and therapeutic ultrasound transducer, which is configured to provide an image of the injury location and ablate and fracture the at least one of cartilage and surrounding tissue in an injury location. In some embodiment, the controller has a display, which can be configured to display the image of the injury location.

In some embodiments, the system includes an optic source contained within the housing. In one embodiment, the optic source can be configured to provide a plurality of images of the injury location to a display. In one embodiment, the optic source can be configured to provide a video of the injury location to a display. In some embodiments, the system can include a monitoring system contained within the housing. In one embodiment, the monitoring system can be configured to monitor a temperature of the cartilage and/or the surrounding tissue in an injury location.

Various embodiments provide a method of non-invasive micro-fraction surgery. The method can include the steps of identifying an injury location comprising cartilage; directing a conformal distribution of ultrasound energy to at least one of cartilage and surrounding subcutaneous tissue in the injury location; ablating the at least one of cartilage and surrounding subcutaneous tissue in the injury location; fracturing a portion of the cartilage in the injury location; initiating regrowth of the cartilage at the injury location; and sparing intervening tissue between a surface of skin above the injury location and the at least one of cartilage and surrounding subcutaneous tissue in the injury location.

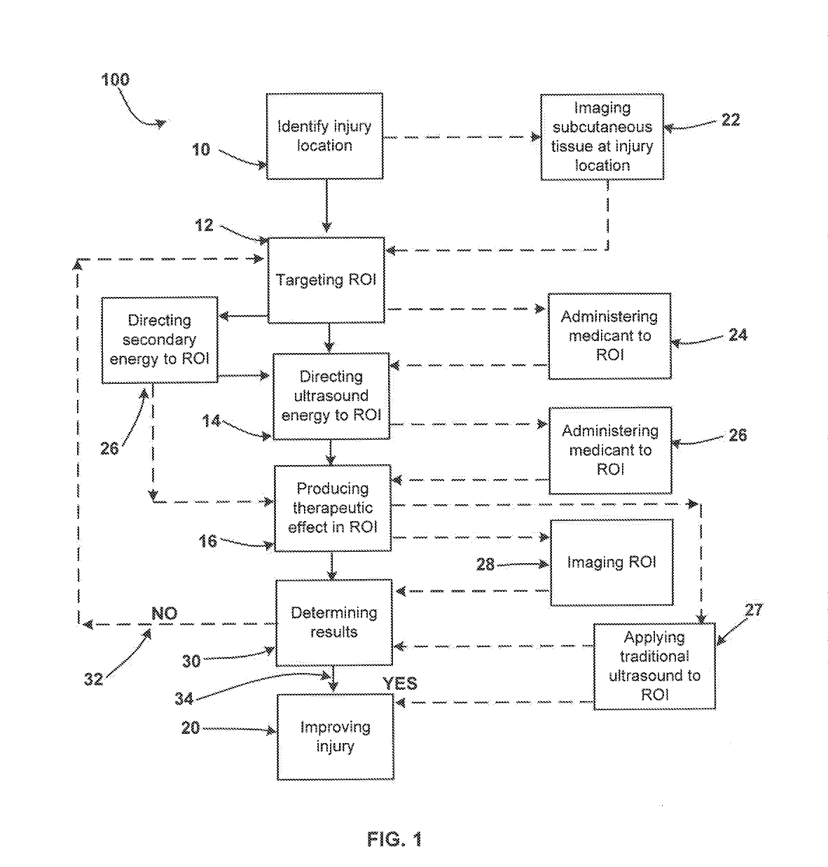

In some embodiments, the method can include the step of welding a portion of the cartilage at the injury location with the conformal distribution of ultrasound energy. In some embodiments, the method can include the step of creating a plurality of micro ablations in at least one of the cartilage and the surrounding subcutaneous tissue in the injury location. In some embodiments, the method can include the step of increasing blood perfusion to the injury location. In some embodiments, the surrounding subcutaneous tissue is bone. In some embodiments, the method includes the step of fracturing a portion of the bone with the conformal distribution of ultrasound energy to initiate re-growth of the cartilage onto the bone. With reference to FIG. 1, a method of treatment is illustrated according to various embodiments. Step 10 is identifying the injury location. The injury location maybe anywhere in the body that contains cartilage, such as, for example, a joint in any of the following: leg, arm, wrist, hand, ankle, knee, foot, hip, shoulder, back, spine, neck, chest, abdomen, and combinations thereof. Next, Step 12 is targeting a region of interest (“ROI”). ROI can be located in subcutaneous tissue below the skin surface of the injury location, which can be anywhere in the body that contains cartilage, such as, those listed previously. In various embodiments, ROI includes cartilage. The muscle and connective layer can comprise cartilage and ROI may also comprise any or all of the following tissues: muscle, tendon, bone, and ligament.

Optionally, step 22 is imaging subcutaneous tissue at the injury location and can be between steps 10 and 12 or can be substantially simultaneous with or be part of step 12. Additionally, imaging may include information from other modalities, such as x-ray, or MRI, which can be imported, linked, or fused. Typically, when the target tissue is cartilage, imaging is used to identify the injury and to direct therapeutic ultrasound energy to precise locations on the cartilage without damaging surrounding tissue.

After step 12, step 14 is directing therapeutic ultrasound energy to ROI. The therapeutic ultrasound energy may be focused or unfocused. The therapeutic ultrasound energy can be focused to the muscle and connective tissue layer. The therapeutic ultrasound energy may ablate a portion of cartilage in the muscle and connective tissue layer. The therapeutic ultrasound energy may coagulate a portion of cartilage in the muscle and connective tissue layer. The therapeutic ultrasound energy can produce at least one lesion in cartilage in the muscle and connective tissue layer. The therapeutic ultrasound energy may micro-score a portion of cartilage in the muscle and connective tissue layer. The therapeutic ultrasound energy may be streaming. The therapeutic ultrasound energy may be directed to a first depth and then directed to a second depth. The therapeutic ultrasound. energy may force a pressure gradient in cartilage in the muscle and connective tissue layer. The therapeutic ultrasound energy may be cavitation. The therapeutic ultrasound energy may be a first ultrasound energy effect, which comprises an ablative or a hemostatic effect, and a second ultrasound energy effect, which comprises at least one of non-thermal streaming, hydrodynamic, diathermic, and resonance induced tissue effects. Directing therapeutic ultrasound energy to ROI is a non-invasive technique. As such, the layers above the muscle and connective tissue layer are spared from injury. In various embodiments, the layers above the targeted cartilage are spared from injury. Such treatment does not require an incision in order to reach the cartilage to perform treatment for the injury. In various embodiments, the therapeutic ultrasound energy level for ablating cartilage in a joint is in the range of about 0.1 joules to about 500 joules in order to create an ablative lesion. However, the therapeutic ultrasound energy 108 level can be in the range of from about 0.1 joules to about 100 joules, or from about 1 joules to about 50 joules, or from about 0.1 joules to about 10 joules, or from about 50 joules to about 100 joules, or from about 100 joules to about 500 joules, or from about 50 joules to about 250 joules. Further, the amount of time therapeutic ultrasound energy is applied at these levels to create a lesion varies in the range from approximately 1 millisecond to several minutes. However, the ranges can be from about 1 millisecond to about 5 minutes, or from about 1 millisecond to about 1 minute, or from about 1 millisecond to about 30 seconds, or from about 1 millisecond to about 10 seconds, or from about 1 millisecond to about 1 second, or from about 1 millisecond to about 0.1 seconds, or about 0.1 seconds to about 10 seconds, or about 0.1 seconds to about 1 second, or from about 1 millisecond to about 200 milliseconds, or from about 1 millisecond to about 0.5 seconds.

The frequency of the ultrasound energy can be in a range from about 0.1 MHz to about 100 MHz, or from about 0.1 MHz to about 50 MHz, or from about 1 MHz to about 50 MHz or about 0.1 MHz to about 30 MHz, or from about 10 MHz to about 30 MHz, or from about 0.1 MHz to about 20 MHz, or from about 1 MHz to about 20 MHz, or from about 20 MHz to about 30 MHz.

The frequency of the ultrasound energy can be in a range from about 1 MHz to about 12 MHz, or from about 5 MHz to about 15 MHz, or from about 2 MHz to about 12 MHz or from about 3 MHz to about 7 MHz.

In some embodiments, the ultrasound energy can be emitted to depths at or below a skin surface in a range from about 0 mm to about 150 mm, or from about 0 mm to about 100 mm, or from about 0 mm to about 50 mm, or from about 0 mm to about 30 mm, or from about 0 mm to about 20 mm, or from about 0 mm to about 10 mm, or from about 0 mm to about 5 mm. In some embodiments, the ultrasound energy can be emitted to depths below a skin surface in a range from about 5 mm to about 150 mm, or from about 5 mm to about 100 mm, or from about 5 mm to about 50 mm, or from about 5 mm to about 30 mm, or from about 5 mm to about 20 mm, or from about 5 mm to about 10 mm. In some embodiments, the ultrasound energy can be emitted to depths below a skin surface in a range from about 10 mm to about 150 mm, or from about 10 mm to about 100 mm, or from about 10 mm to about 50 mm, or from about 10 mm to about 30 mm, or from about 10 mm to about 20 mm, or from about 0 mm to about 10 mm.

In some embodiments, the ultrasound energy can be emitted to depths at or below a skin surface in the range from about 20 mm to about 150 mm, or from about 20 mm to about 100 mm, or from about 20 mm to about 50 mm, or from about 20 mm to about 30 mm. In some embodiments, the ultrasound energy can be emitted to depths at or below a skin surface in a range from about 30 mm to about 150 mm, or from about 30 mm to about 100 mm, or from about 30 mm to about 50 mm. In some embodiments, the ultrasound energy can be emitted to depths at or below a skin surface in a range from about 50 mm to about 150 mm, or from about 50 mm to about 100 mm. In some embodiments, the ultrasound energy can be emitted to depths at or below a skin surface in a range from about 20 mm to about 60 mm, or from about 40 mm to about 80 mm, or from about 10 mm to about 40 mm, or from about 5 mm to about 40 mm, or from about 0 mm to about 40 mm, or from about 10 mm to about 30 mm, or from about 5 mm to about 30 mm, or from about 0 mm to about 30 mm.

In various embodiments, a temperature of tissue receiving the ultrasound energy can be in a range from 30° C. to about 100° C., or from 43° C. to about 60° C., or from 50° C. to about 70° C., or from 30° C. to about 50° C., or from 43° C. to about 100° C., or from 33° C. to about 100° C., or from 30° C. to about 65° C., or from 33° C. to about 70° C., as well as variations thereof. Alternatively, the targeted skin surface and the layers above a target point in the subcutaneous layer are heated to a 10° C. to 15° C. above the tissue's natural state.

In various embodiments, the ultrasound energy may be emitted at various energy levels, such as for example, the energy levels described herein. Further, the amount of time ultrasound energy is applied at these levels for various time ranges, such as for example, the ranges of time described herein. The frequency of the ultrasound energy is in various frequency ranges, such as for example, the frequency ranges described herein. The ultrasound energy can be emitted to various depths below a targeted skin surface, such as for example, the depths described herein.

Optionally, step 24, which is administering a medicant to ROI, can be between steps 12 and 14. The medicant can be any chemical or naturally occurring substance that can assist in treating the injury. For example the medicant can be but not limited to a pharmaceutical, a drug, a medication, a nutriceutical, an herb, a vitamin, a cosmetic, an amino acid, a collagen derivative, a holistic mixture, an anti-inflammant, a steroid, a blood vessel dilator or combinations thereof.

The medicant can be administered by applying it to the skin above ROI. The medicant can be administered to the circulatory system. For example, the medicant can be in the blood stream and can be activated or moved to ROI by the ultrasound energy. The medicant can be administered by injection into or near ROI. Any naturally occurring proteins, stem cells, growth factors and the like can be used as medicant in accordance to various embodiments. A medicant can be mixed in a coupling gel or can be used as a coupling gel.

Step 16 is producing a therapeutic effect in ROI. A therapeutic effect can be cauterizing and repairing a portion of cartilage in the muscle and connective tissue layer. A therapeutic effect can be stimulating or increase an amount of heat shock proteins. Such a therapeutic effect can cause white blood cells to promote healing of a portion of cartilage in the muscle and connective layer in the ROI. A therapeutic effect can be peaking inflammation in a portion of the ROI to decrease pain at the injury location. A therapeutic effect can be creating lesion to restart or increase the wound healing cascade at the injury location. A therapeutic effect can be increasing the blood perfusion to the injury location. Such a therapeutic effect would not require ablative ultrasound energy. A therapeutic effect can be encouraging collagen growth. A therapeutic effect can be relieving pain. A therapeutic effect may increase the “wound healing” response through the liberation of cytokines and may produce reactive changes within the tendon and muscle itself, helping to limit surrounding tissue edema and decrease an inflammatory response to an injury to a joint.

A therapeutic effect can be synergetic with the medicant administered to ROI in steps 24 and/or 26. A therapeutic effect may be an enhanced delivery of a medicant administered to ROI in steps 24 and/or 26. A therapeutic effect may increase an amount of a medicant administered to ROI in steps 24 and/or 26. A therapeutic effect may be stimulation of a medicant administered to ROI in steps 24 and/or 26. A therapeutic effect may be initiation of a medicant administered to ROI in steps 24 and/or 26. A therapeutic effect may be potentiation of a medicant administered to ROI in steps 24 and/or 26.

A therapeutic effect can be healing an injury to a muscle. A therapeutic effect can be repairing a tendon. A therapeutic effect can be repairing a ligament. A therapeutic effect can be regenerating cartilage. A therapeutic effect can be removing damaged cartilage. A therapeutic effect can be repairing cartilage in a joint. Therapeutic effects can be combined.

A therapeutic effect can be produced by a biological effect that initiated or stimulated by the ultrasound energy. A biological effect can be stimulating or increase an amount of heat shock proteins. Such a biological effect can cause white blood cells to promote healing of a portion of cartilage in the muscle and connective tissue layer. A biological effect can be to restart or increase the wound healing cascade at the injury location. A biological effect can be increasing the blood perfusion to the injury location. A biological effect can be encouraging collagen growth at the injury location. A biological effect may increase the liberation of cytokines and may produce reactive changes within a portion of cartilage in the muscle and connective tissue layer. A biological effect may by peaking inflammation in a portion of cartilage in the muscle and connective tissue layer. A biological effect may at least partially shrinking collagen in a portion of cartilage in the muscle and connective tissue layer. A biological effect may be denaturing of proteins in ROI.

A biological effect may be creating immediate or delayed cell death (apoptosis) in the injury location. A biological effect may be collagen remodeling in the injury location. A biological effect may be the disruption or modification of biochemical cascades in the injury location. A biological effect may be the production of new collagen in the injury location. A biological effect may a stimulation of cell growth in the injury location. A biological effect may be angiogenesis in the injury location. A biological effect may a cell permeability response in the injury location.

A biological effect may be an enhanced delivery of a medicant to the injury location. A biological effect may increase an amount of a medicant in the injury location. A biological effect may be stimulation of a medicant in the injury location. A biological effect may be initiation of a medicant in the injury location. A biological effect may be potentiation of a medicant in the injury location.

Optionally, step 26, which is administering medicant to ROI, can be between steps 14 and 16 or can be substantially simultaneous with or be part of step 16. The medicants useful in step 26 are essentially the same as those discussed for step 24.

In various embodiments, ultrasound energy is deposited, which can stimulate a change in at least one of concentration and activity in the injury location of one or more of the following: Adrenomedullin (AM), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colonystimulating factor (0-CSF), Granulocyte macrophage colony-stimulating factor (OMCSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TPO), Transforming growth factor alpha(TGF-α), Transforming growth factor beta(TGF-β), Tumor necrosis factor-alpha(TNF-α), Vascular endothelial growth factor (VEGF), Wnt Signaling Pathway, placental growth factor (PIGF), [(Foetal Bovine Somatotrophin)] (FBS), IL-1-Cofactor for IL-3 and IL-6, which can activate T cells, IL-2-T-cell growth factor, which can stimulate IL-1 synthesis and can activate B cells and NK cells, IL-3, which can stimulate production of all non-lymphoid cells, IL-4-Growth factor for activating B cells, resting T cells, and mast cells, IL-5, which can induce differentiation of activated B cells and eosinophils, IL-6, which can stimulate Ig synthesis and growth factor for plasma cells, IL-7 growth factor for pre-B cells, and/or any other growth factor not listed herein, and combinations thereof.

Further, medicants, as described above, can include a drug, a medicine, or a protein, and combinations thereof. Medicants can also include adsorbent chemicals, such as zeolites, and other hemostatic agents are used in sealing severe injuries quickly. Thrombin and fibrin glue are used surgically to treat bleeding and to thrombose aneurysms. Medicants can include Desmopressin is used to improve platelet function by activating arginine vasopressin receptor 1A. Medicants can include coagulation factor concentrates are used to treat hemophilia, to reverse the effects of anticoagulants, and to treat bleeding in patients with impaired coagulation factor synthesis or increased consumption. Prothrombin complex concentrate, cryoprecipitate and fresh frozen plasma are commonly-used coagulation factor products. Recombinant activated human factor VII can be used in the treatment of major bleeding. Medicants can include tranexamic acid and aminocaproic acid, can inhibit fibrinolysis, and lead to a de facto reduced bleeding rate. In addition, medicants can include steroids like the glucocorticoid cortisol.

Optionally, after step 12, step 25, which is directing secondary energy to ROI, can be substantially simultaneous with or be part of step 16. However, step 25 can be administered at least one of before and after step 16. Step 25 can be alternated with step 16, which can create a pulse of two different energy emissions to ROI. Secondary energy cart be provided by a laser source, or an intense pulsed light source, or a light emitting diode, or a radio frequency, or a plasma source, or a magnetic resonance source, or a mechanical energy source, or any other photon-based energy source. Secondary energy can be provided by any appropriate energy source now known or created in the future. More than one secondary energy source may be used for step 25.

Furthermore, various embodiments provide energy, which may be a first energy and a second energy. For example, a first energy may be followed by a second energy, either immediately or after a delay period. In another example, a first energy and a second energy can be delivered simultaneously. In one embodiment, the first energy and the second energy is ultrasound energy. In some embodiments, the first energy is ultrasound and the second energy is generated by one of a laser, an intense pulsed light, a light emitting diode, a radiofrequency generator, photon-based energy source, plasma source, a magnetic resonance source, or a mechanical energy source, such as for example, pressure, either positive or negative. In other embodiments, energy may be a first energy, a second energy, and a third energy, emitted simultaneously or with a time delay or a combination thereof. In one embodiment, energy may be a first energy, a second energy, a third energy, and an nth energy, emitted simultaneously or with a time delay or a combination thereof.

Any of the a first energy, a second energy, a third energy, and an nth energy may be generated by at least one of a laser, an intense pulsed light, a light emitting diode, a radiofrequency generator, an acoustic source, photon-based energy source, plasma source, a magnetic resonance source, and/or a mechanical energy source.

Step 20 is improving the injury. Optionally, between steps 16 and 20 is step 30, which is determining results. Results may be repairing cartilage. Results may be completing a micro-fracture procedure. Results may be regenerating cartilage. Between steps 16 and 30 is option step 28, which is imaging ROI. The images of ROI from step 28 can be useful for the determining results of step 30. If the results of step 30 are acceptable within the parameters of the treatment then Yes direction 34 is followed to step 20. If the results of step 30 are not acceptable within the parameters of the treatment then No direction 32 is followed back to step 12. After step 16, optionally traditional ultrasound heating can be applied to ROI in step 27. This application of traditional ultrasound heating to ROI can be useful in keeping a medicant active or providing heat to support blood perfusion to ROI after step 16. Further examples and variations of treatment method 100 are discussed herein.

In addition, various different subcutaneous tissues, including for example, cartilage, may be treated by method 100 to produce different bio-effects, according to some embodiments of the present disclosure. Furthermore, any of portion of a joint may be treated by method 100 to produce one or more bio-effects, as described herein, in accordance to various embodiments. In order to treat a specific injury location and to achieve a desired bio-effect, therapeutic ultrasound energy may be directed to a specific depth within ROI to reach the targeted subcutaneous tissue, such as, for example, cartilage. For example, if it is desired to cut cartilage by applying therapeutic ultrasound energy at ablative levels, which may be approximately 5 mm to 15 mm below skin surface or at other depths as described herein. An example of ablating cartilage can include a series of lesions ablated into muscle. Besides ablating a portion of cartilage in the joint, other bio-effects may comprise incapacitating, partially incapacitating, severing, rejuvenating, removing, ablating, micro-ablating, shortening, manipulating, or removing, tissue either instantly or over time, and combinations thereof.

Depending at least in part upon the desired bio-effect and the subcutaneous tissue being treated, method 100 may be used with an extracorporeal, non-invasive procedure. Also, depending at least in part upon the specific bio-effect and tissue targeted, temperature may increase within ROI may range from approximately 30° C. to about 60° C., or in a range from about 30° C. to about 100° C., or in other appropriate temperature ranges that are described herein. In order to treat a specific injury location and to achieve a desired bio-effect, therapeutic ultrasound energy may be directed to a specific depth within ROI to reach the targeted cartilage. Depending at least in part upon the desired bio-effect and the subcutaneous tissue being treated, method 100 may be used with an extracorporeal, non-invasive procedure. Also, depending at least in part upon the specific bio-effect and tissue targeted, temperature may increase within ROI may range from approximately 30° C. to about 60° C., or in a range from about 30° C. to about 100° C., or in other appropriate temperature ranges that are described herein. Also, depending at least in part upon the specific bio-effect and tissue targeted, temperature may increase within ROI may range from approximately 1 10° C. to about 15° C.

Other bio-effects to target tissue, such as, a portion of tissue in the joint, can include heating, cavitation, streaming, or vibro-accoustic stimulation, and combinations thereof. In various embodiments, therapeutic ultrasound energy is deposited in a matrices of micro-coagulative zones to an already injured tendon, muscle, and/or cartilage can increase the “wound healing” response through the liberation of cytokines and may produce reactive changes within the tendon, muscle, and/or cartilage itself, helping to limit surrounding tissue edema and decrease the inflammatory response to an injury to a joint. In various embodiments, therapeutic ultrasound energy is deposited in a matrices of micro-coagulative zones to an already injured tendon, muscle, and/or cartilage changes at least one of concentration and activity of inflammatory mediators (such as but not limited to TNF-A, IL-1) as well as growth factors (such as but not limited to TGF-B1, TGF-B3) at the site of the injured tendon, muscle, and/or cartilage.

In various embodiments, therapeutic ultrasound energy is deposited in a matrices of micro-coagulative zones to an already injured tendon, muscle, and/or cartilage which can stimulate a change in at least one of concentration and activity of one or more of the following: Adrenomedullin (AM), Autocrine motility factor, Bone morphogenetic proteins (BMPs), Brain-derived. neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GMCSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TP0), Transforming growth factor alpha(TGF-α), Transforming growth factor beta(TGF-β), Tumour necrosis factor-alpha(TNF-α), Vascular endothelial growth factor (VEGF), Wnt Signaling Pathway, placental growth factor (PIGF), [(Foetal Bovine Somatotrophin)] (FBS), IL-1-Cofactor for IL-3 and IL-6, which can activate T cells, IL-2-T-cell growth factor, which can stimulate IL-1 synthesis and can activate B cells and NK cells, IL-3, which can stimulate production of all non-lymphoid cells, IL-4-Growth factor for activating B cells, resting T cells, and mast cells, IL-5, which can induce differentiation of activated B cells and eosinophils, IL-6, which can stimulate Ig synthesis and growth factor for plasma cells, IL-7 growth factor for pre-B cells, and/or any other growth factor not listed herein, and combinations thereof.

Further, medicants, as described above, can include a drug, a medicine, or a protein, and combinations thereof. Medicants can also include adsorbent chemicals, such as zeolites, and other hemostatic agents are used in sealing severe injuries quickly. Thrombin and fibrin glue are used surgically to treat bleeding and to thrombose aneurysms. Medicants can include Desmopressin is used to improve platelet function by activating arginine vasopressin receptor 1A. Medicants can include coagulation factor concentrates are used to treat hemophilia, to reverse the effects of anticoagulants, and to treat bleeding in patients with impaired coagulation factor synthesis or increased consumption. Prothrombin complex concentrate, cryoprecipitate and fresh frozen plasma are commonly-used coagulation factor products. Recombinant activated humari factor VII can be used in the treatment of major bleeding. Medicants can include tranexamic acid and aminocaproic acid, can inhibit fibrinolysis, and lead to a de facto reduced bleeding rate. In addition, medicant can include steroids, (anabolic steroids and/or cortisol steroids), for example glucocorticoid cortisol or prednisone. Medicant can include compounds as alpha lipoic acid, DMAE, vitamin C ester, tocotrienols, and phospholipids.

Medicant can be a pharmaceutical compound such as for example, cortisone, Etanercept, Abatacept, Adalimumab, or Infliximab. Medicant can include plateletrich plasma (PRP), mesenchymal stem cells, or growth factors. For example, PRP is typically a fraction of blood that has been centrifuged. The PRP is then used for stimulating healing of the injury. The PRP typically contains thrombocytes (platelets) and cytokines (growth factors). The PRP may also contain thrombin and may contain fibenogen, which when combined can form fibrin glue. Medicant can be a prothrombin complex concentrate, cryoprecipitate and fresh frozen plasma, which are commonly-used coagulation factor products. Medicant can be a recombinant activated human factor VII, which can be used in the treatment of major bleeding. Medicant can include tranexamic acid and aminocaproic acid, can inhibit fibrinolysis, and lead to a de facto reduced bleeding rate. In some embodiments, medicant can be Botox.

According to various embodiments of method 100, ultrasound probe is coupled directly to ROI, as opposed to skin surface, to treat targeted tissue. For example, ultrasound probe can be integrated to or attached to a tool, such as, for example, an arthroscopic tool, laparoscopic tool, or an endoscopic tool that may be inserted into a patient's body with minimal invasiveness. In various embodiments, method 100 can treat either recent or older injuries, or combinations thereof. Inflammation can be classified as either acute or chronic. Acute inflammation is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process. In various embodiments, method 100 can treat chronic inflammation. In various embodiments, method 100 can treat acute inflammation. In some embodiments, method 100 can treat a combination of acute and chronic inflammation.

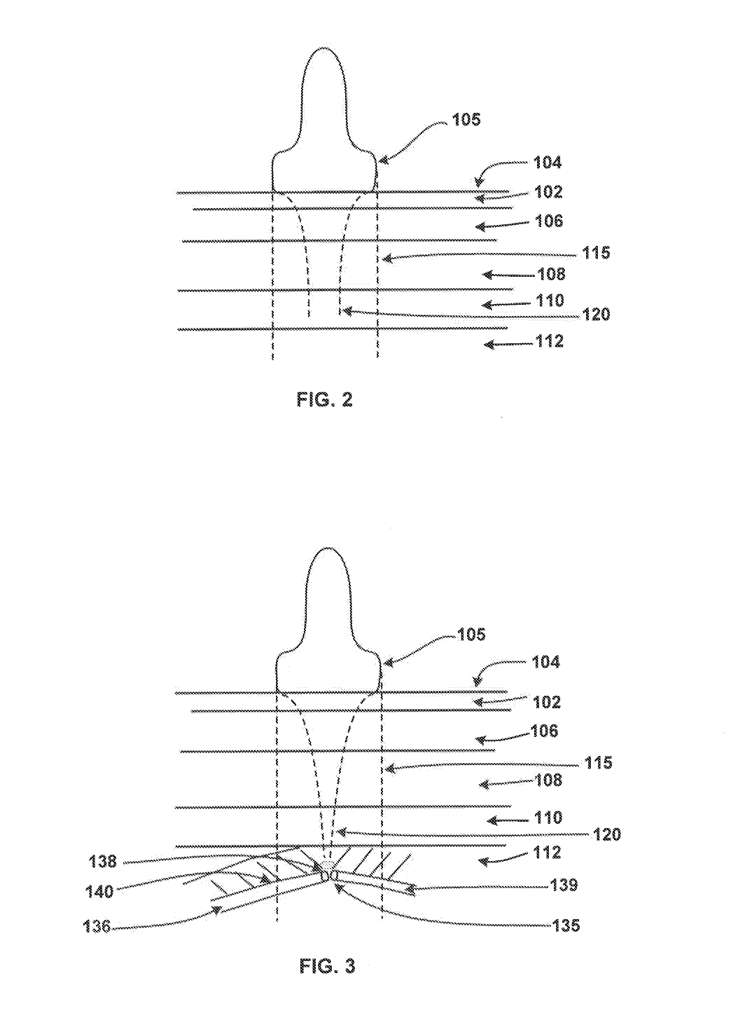

Now moving to FIG. 2, a cross sectional view of tissue layers and ultrasound energy directed to a muscle and connective tissue layer, according to various embodiments, is illustrated. In various embodiments, ultrasound energy 120 creates a conformal region of elevated temperature. In some embodiments, conformal region of elevated temperature is a conformal energy deposition, which increases the temperature in a conformal region of tissue in ROI 115 by about 5° C. to 65° C. above the internal body temperature or higher. In some embodiments, conformal region of elevated temperature is a conformal energy deposition, which is placed at a selected depth in the tissue in ROI 115 and has a defined shape and volume. In some embodiments, conformal region of elevated temperature is a shaped conformal distribution of elevated temperature in ROI 115, which can be created through adjustment of the strength, depth, and type of focusing, energy levels and timing cadence.

In various embodiment, ultrasound probe 105 is configured with the ability to controllably produce conformal distribution of elevated temperature in soft tissue within ROI 115 through precise spatial and temporal control of acoustic energy deposition, i.e., control of ultrasound probe 105 is confined within selected time and space parameters, with such control being independent of the tissue. The ultrasound energy 120 can be controlled to produce a conformal distribution of elevated temperature in soft tissue within ROI 115 using spatial parameters. The ultrasound energy 120 can be controlled to produce conformal distribution of elevated temperature in soft tissue within ROI 115 using temporal parameters. The ultrasound energy 120 can be controlled to produce a conformal distribution of elevated temperature in soft tissue within ROI 115 using a combination of spatial parameters and temporal parameters. In some embodiments, a conformal distribution of elevated temperature in soft tissue within ROI 115 is conformal region of elevated temperature in ROI 115.

In various embodiments, conformal region of elevated temperature can create a lesion in ROI 115. In various embodiments, conformal region of elevated temperature can initiate thermal injury in a portion of ROI 115. In various embodiments, conformal region of elevated temperature can initiate or stimulate coagulation in a portion of ROI 115. In various embodiments, conformal region of elevated temperature can be one of a series of micro scoring in ROI 115. In various embodiments, conformal region of elevated temperature can with a first ultrasound energy deposition and a second energy deposition. In one embodiment, second energy deposition is ultrasound energy. In some embodiments, second energy is any one of second energy that may be used for method 100, as discussed herein. In various embodiments, conformal region of elevated temperature can stimulate and/or initiate a therapeutic effect. In various embodiments, conformal region of elevated temperature can stimulate and/or initiate a biological effect. In various embodiments, conformal region of elevated temperature can denature tissue in ROI 115. In various embodiments, conformal region of elevated temperature can drive a medicant into ROI 115. In various embodiments, conformal region of elevated temperature can activate a medicant in ROI 115. In various embodiments, conformal region of elevated temperature can create immediate or delayed cell death (apoptosis) in the ROI. In various embodiments, conformal region of elevated temperature can create one or more ablation zones in ROI 115. In various embodiments, conformal region of elevated temperature can increase blood perfusion in ROI 115. In one embodiment, conformal region of elevated temperature can be created by heating a portion of ROI 115 with ultrasound energy 120. In one embodiment, conformal region of elevated temperature can be created by cavitation in ROI 115, which is initiated by ultrasound energy 120. In one embodiment, conformal region of elevated temperature can be created by streaming ultrasound energy 120 into ROI 115. In one embodiment, conformal region of elevated temperature can be created by vibro-accoustic stimulation in ROI 115, which is initiated by ultrasound energy 120. In one embodiment, conformal region of elevated temperature can be created by a combination of two or more of heating, cavitation, streaming, or vibro-accoustic stimulation.

In some embodiments, conformal region of elevated temperature can be a shaped lesion, which can be created through adjustment of the, strength, depth, and type of focusing, energy levels and timing cadence. For example, focused ultrasound energy 120 can be used to create precise arrays of microscopic thermal ablation zones. Ultrasound energy 120 can produce an array of ablation zones deep into the layers of the soft tissue. Detection of changes in the reflection of ultrasound energy can be used for feedback control to detect a desired effect on the tissue and used to control the exposure intensity, time, and/or position. In various embodiments, ultrasound probe 105 is configured with the ability to controllably produce conformal region of elevated temperature in soft tissue within ROI 115 through precise spatial and temporal control of acoustic energy deposition, i.e., control of ultrasound probe 105 is confined within selected time and space parameters, with such control being independent of the tissue.

In accordance with various embodiments, ultrasound probe 105 can be configured for spatial control of ultrasound energy 120 by controlling the manner of distribution of the ultrasound energy 120 to create conformal region of elevated temperature. For example, spatial control may be realized through selection of the type of one or more spatial parameters of the transducer configurations of ROI 115, selection of the placement and location of ultrasound probe 105 for delivery of ultrasound energy 120 relative to ROI 115 e.g., ultrasound probe 105 being configured for scanning over part or whole of ROI 115 to produce a contiguous conformal region of elevated temperature having a particular orientation or otherwise change in distance from ROI 115, and/or control of other environment parameters, e.g., the temperature at the acoustic coupling interface can be controlled, and/or the coupling of ultrasound probe 105 to tissue. Other spatial control can include but are not limited to geometry configuration of ultrasound probe 105 or transducer assembly, lens, variable focusing devices, variable focusing lens, stand-offs, movement of ultrasound probe, in any of six degrees of motion, transducer backing, matching layers, number of transduction elements in transducer, number of electrodes, or combinations thereof.

In various embodiments, ultrasound probe 105 can also be configured for temporal control of ultrasound energy 120 by controlling the timing of the distribution of the ultrasound energy 120 to create conformal region of elevated temperature. For example, temporal control may be realized through adjustment and optimization of one or more temporal parameters, such as for example, drive amplitude levels, frequency, waveform selections, e.g., the types of pulses, bursts or continuous waveforms, and timing sequences and other energy drive characteristics to control thermal ablation of tissue. Other temporal parameters can include but are not limited to full power burst of energy, shape of burst, timing of energy bursts, such as, pulse rate duration, continuous, delays, etc., change of frequency of burst, burst amplitude, phase, apodization, energy level, or combinations thereof. The spatial and/or temporal control can also be facilitated through open-loop and closed-loop feedback arrangements, such as through the monitoring of various spatial and temporal characteristics. As a result, control of acoustical energy within six degrees of freedom, e.g., spatially within the X, Y and Z domain, as well as the axis of rotation within the XY, YZ and XZ domains, can be suitably achieved to generate conformal region of elevated temperature of variable shape, size and orientation. For example, through such spatial and/or temporal control, ultrasound probe 105 can enable the regions of thermal injury to possess arbitrary shape and size and allow the tissue to be destroyed (ablated) in a controlled manner.

The tissue layers illustrated in FIG. 2 are skin surface 104, epidermal layer 102, dermis layer 106, fat layer 108, SMAS layer 110, and muscle and connective tissue layer 112. In some embodiments, muscle and connective tissue layer 112 comprises cartilage. Ultrasound probe 105 emits therapeutic ultrasound energy 120 in ROI 115. In various embodiments, ultrasound probe 105 is capable of emitting therapeutic ultrasound energy 120 at variable depths in ROI 115, such as, for example, the depths described herein. Ultrasound probe 105 is capable of emitting therapeutic ultrasound energy as a single frequency, variable frequencies, or a plurality of frequencies, such as, for example, the frequency ranges described herein. Ultrasound probe 105 is capable of emitting therapeutic ultrasound energy 120 for variable time periods or to pulse the emission over time, such as, for example, those time intervals described herein. Ultrasound probe 105 is capable of providing various energy levels of therapeutic ultrasound energy, such as, for example, the energy levels described herein. Ultrasound probe 105 may be individual hand-held device, or may be part of a treatment system. The ultrasound probe 105 can provide both therapeutic ultrasound energy and imaging ultrasound energy. However, ultrasound probe 105 may provide only therapeutic ultrasound energy. Ultrasound probe 105 may comprise a therapeutic transducer and a separate imaging transducer. Ultrasound probe 105 may comprise a transducer or a transducer array capable of both therapeutic and imaging applications. In some embodiments, ultrasound probe 105 emit therapeutic ultrasound energy 120, which creates a conformal region of elevated temperature in ROI 115. In various embodiments, ultrasound probe 105 may be used for method 100. In various embodiments, method 100 can be implemented using any or all of the elements illustrated in FIG. 2. As will be appreciated by those skilled in the art, at least a portion of method 100 or a variation of method 100 can be implemented using any or all of the elements illustrated in FIG. 2.

In FIG. 3, a cross sectional view of tissue layers and ultrasound energy directed to at least one of cartilage 140 and ligament 138, according to various embodiments, is illustrated. The tissue layers illustrated are skin surface 104, epidermal layer 102, dermis layer 106, fat layer 108, SMAS layer 110, and muscle and connective tissue layer 112, which comprises cartilage 140 and ligament 138. As well known to those skilled in the art, joint 135 can comprise ligament 138, cartilage 140, and bone 136. In some embodiments, ROI 115 comprises at least one of cartilage 140 and ligament 138. In some embodiments, ROI 115 can comprise at least a portion of joint 135. ROI 115 can comprise any or all of the following: skin surface 104, epidermal layer 1 02, dermis layer 106, fat layer 108, SMAS layer 110, and muscle and connective tissue 112, which comprises ligament 138 and cartilage 140. In some embodiments, ultrasound probe 105 can image at least a portion of one of skin surface 104, epidermal layer 102, dermis layer 106, fat layer 108, SMAS layer 110, ligament 13 8 and cartilage 140. Ultrasound probe 105 emits therapeutic ultrasound energy 120 to at least one of ligament 138 and cartilage 140. In various embodiments, therapeutic ultrasound energy 120 treats at least one of ligament 138 and cartilage 140. In various embodiments, therapeutic ultrasound energy 120 treats at least a portion of joint 135. In various embodiments, ultrasound probe 105 may be used for method 100. In various embodiments, method 100 can be implemented using any or all of the elements illustrated in FIG. 3. As will be appreciated by those skilled in the art, at least a portion of method 190 or a variation of method 100 can be implemented using any or all of the elements illustrated in FIG. 3.

In one embodiment, therapeutic ultrasound energy 120 ablates a portion of cartilage 140 creating a lesion. In one embodiment, therapeutic ultrasound energy 120 ablates a portion of joint 135 creating a lesion. In one embodiment therapeutic ultrasound energy coagulates a portion of cartilage 140. In one embodiment therapeutic ultrasound energy 120 coagulates a portion of joint 135. In some embodiments, therapeutic ultrasound energy 120 regenerates cartilage 140. In one embodiment, therapeutic ultrasound energy 120 ablates a portion of cartilage 140. In one embodiment, therapeutic ultrasound energy 120 increases perfusion of blood to a portion of cartilage 140. In one embodiment, therapeutic ultrasound energy 120 welds damaged cartilage 140 to repair a tear in cartilage 140.

In some embodiments, ultrasound probe 105 can be moved in at least one direction to provide a plurality of lesions in cartilage 140. In various embodiments, a plurality of lesions can be placed in a pattern in a portion of cartilage 140, such as, for example, a 1-D pattern, a 2-D pattern, a 3-D pattern, or combinations thereof. In one embodiment, therapeutic ultrasound energy 120 ablates a portion muscle 130 creating lesion. In one embodiment, therapeutic ultrasound energy 120 ablates a portion muscle 130 creating lesion. In one embodiment, therapeutic ultrasound energy 120 coagulates a portion of muscle 130. Therapeutic ultrasound energy 120 creates ablation zone in a tissue layer, at which a temperature of tissue is raised to at least 43° C., or is raised to a temperature in the range form about 43° C. to about 100° C., or from about 50° C. to about 90° C., or from about 55° C. to about 75° C., or from about 50° C. to about 65° C., or from about 60° C. to In some embodiments, ultrasound probe 105 can be moved in at least one direction to provide a plurality of lesions in a tissue layer. In various embodiments, a plurality of lesions can be placed in a pattern in at least one tissue layer, such as, for example, a 1-D pattern, a 2-D pattern, a 3-D pattern, or combinations thereof. In one embodiment, ultrasound probe 105 comprises a single transducer element and while emitting therapeutic ultrasound energy 120 in a pulsed matter, is moved in a linear motion along skin surface 104 to create a 1-D pattern of a plurality of lesions in at least one tissue layer. In one embodiment, ultrasound probe 105 comprises a linear array of transducers and while emitting therapeutic ultrasound energy 120 in a pulsed matter, is moved along the linear vector of the array on skin surface 104 to create a 1-D pattern of a plurality of lesions in at least one tissue layer. In one embodiment, ultrasound probe 105 comprises a linear array of transducers and while emitting therapeutic ultrasound energy 120 in a pulsed matter, is moved along the non-linear vector of the array on skin surface 104 to create a 2-D pattern of a plurality of lesions in at least one tissue layer. In one embodiment, ultrasound probe 105 comprises an array of transducers and while emitting therapeutic ultrasound energy 120 in a pulsed matter, is moved along skin surface 104 to create a 2-D pattern of a plurality of lesions in at least one tissue layer.

In one embodiment, ultrasound probe 105 comprises an array of transducers, wherein the array comprises a first portion focusing to a first depth and a second portion focusing to a second depth, and while emitting therapeutic ultrasound energy 120 in a pulsed matter, is moved along skin surface 104 to create a 3-D pattern of a plurality of lesions in at least one tissue layer. In one embodiments, ultrasound probe 105 comprises at least two arrays of transducers, wherein a first array focusing to a first depth and a second array focusing to a second depth, and while each of the arrays emitting therapeutic ultrasound energy 120 in a pulsed matter, is moved along skin surface 104 to create a 3-D pattern of a plurality of lesions in at least one tissue layer.

In one embodiment, ultrasound probe 105 comprises a linear array of transducers and while emitting therapeutic ultrasound energy 120 in a pulsed matter, is moved along the non-linear vector of the array on skin surface 104 focused to a first depth then moved in the same direction along skin surface focused at a second depth to create a 3-D pattern of a plurality of lesions in at least one tissue layer. In one embodiment, ultrasound probe 105 comprises an array of transducers and while emitting therapeutic ultrasound energy 120 in a pulsed matter, is moved along skin surface 104 focused to a first depth then moved in the same direction along skin surface focused at a second depth to create a 3-D pattern of a plurality of lesions in at least one tissue layer.

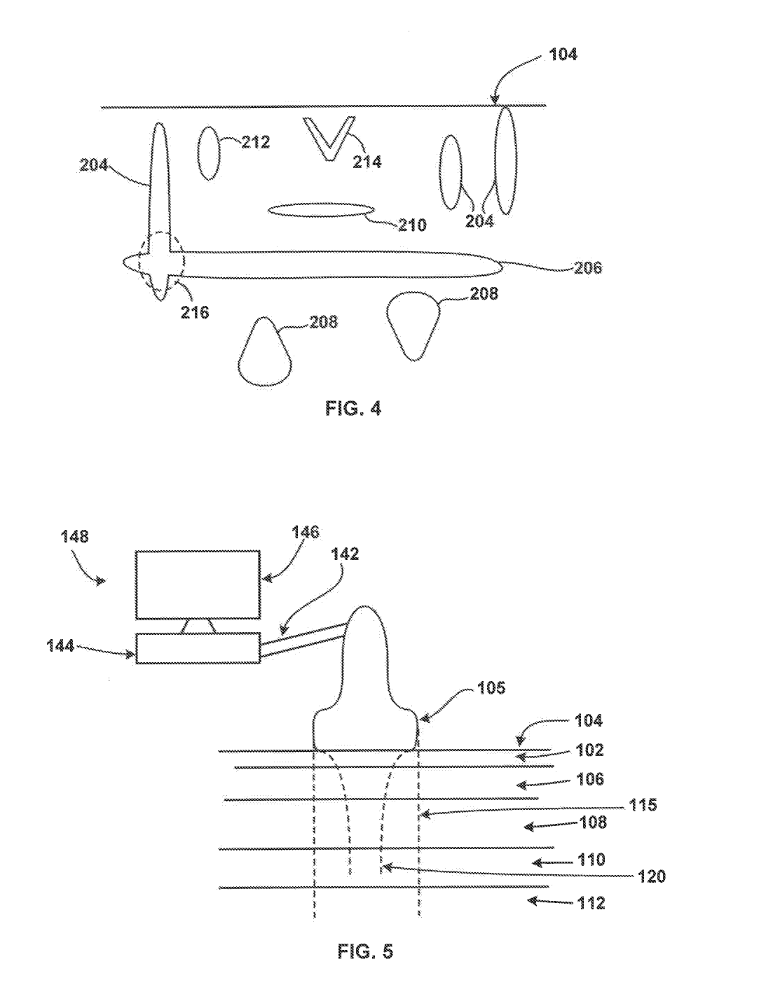

Referring to FIG. 4, various shapes of lesions, according to various embodiments, are illustrated. In various embodiment, ultrasound probe 105 is configured with the ability to controllably produce conformal lesions of thermal injury in muscle and connective tissue layer 112 within ROI 115 through precise spatial and temporal control of acoustic energy deposition, i.e., control of ultrasound probe 105 is confined within selected time and space parameters, with such control being independent of the tissue. In some embodiments, ultrasound probe 105 configured with the ability to controllably a conformal distribution of ultrasound energy. In one embodiment, conformal distribution of ultrasound energy can create a conformal lesion of thermal injury in subcutaneous tissue in ROI 115, for example in muscle and connective tissue layer 112 within ROI 115. In one embodiment, conformal distribution of ultrasound energy can create a conformal region of elevated temperature in subcutaneous tissue in ROI 115, for example in muscle and connective tissue layer 112 within ROI 115.

In accordance with one embodiment, control system and ultrasound probe 105 can be configured for spatial control of therapeutic ultrasound energy 120 by controlling the manner of distribution of the therapeutic ultrasound energy 120. For example, spatial control may be realized through selection of the type of one or more transducer configurations insonifying ROI 115, selection of the placement and location of ultrasound probe 105 for delivery of therapeutic ultrasound energy 120 relative to ROI 115 e.g., ultrasound probe 105 being configured for scanning over part or whole of ROI 115 to produce contiguous thermal injury having a particular orientation or otherwise change in distance from ROI 115, and/or control of other environment parameters, e.g., the temperature at the acoustic coupling interface can be controlled, and/or the coupling of ultrasound probe 105 to human tissue.

In addition to the spatial control parameters, control system and ultrasound probe 105 can also be configured for temporal control, such as through adjustment and optimization of drive amplitude levels, frequency/waveform selections, e.g., the types of pulses, bursts or continuous waveforms, and timing sequences and other energy drive characteristics to control thermal ablation of tissue. The spatial and/or temporal control can also be facilitated through open-loop and closed-loop feedback arrangements, such as through the monitoring of various spatial and temporal characteristics. As a result, control of acoustical energy within six degrees of freedom, e.g., spatially within the X, Y and Z domain, as well as the axis of rotation within the XY, YZ and XZ domains, can be suitably achieved to generate conformal lesions of variable shape, size and orientation.

For example, through such spatial and/or temporal control, ultrasound probe 105 can enable the regions of thermal injury to possess arbitrary shape and size and allow the tissue to be destroyed (ablated) in a controlled manner. With reference to FIG. 4, one or more thermal lesions may be created within muscle and connective tissue layer 112, with such thermal lesions having a narrow or wide lateral extent, long or short axial length, and/or deep or shallow placement in muscle and connective tissue 112. For example, cigar or line-shaped shaped lesions may be produced in a vertical disposition 204 and/or horizontal disposition 206. In addition, raindrop shaped lesions 208, flat planar lesions 210, round lesions 212 and/or other v-shaped/ellipsoidal lesions 214 may be formed, among others. For example, mushroom-shaped lesion may be provided, such as through initial generation of an initial round or cigar-shaped lesion, with continued application of therapeutic ultrasound energy 120 resulting in thermal expansion to further generate a growing lesion 224, such thermal expansion being continued until mushroom-shaped lesion 220 is achieved. The plurality of shapes can also be configured in various sizes and orientations, e.g., lesions 208 could be rotationally oriented clockwise or counterclockwise at any desired angle, or made larger or smaller as selected, all depending on spatial and/or temporal control. Moreover, separate islands of destruction, i.e., multiple lesions separated throughout muscle and connective tissue layer 112, may also be created over part of or the whole portion within ROI 115. In addition, contiguous structures and/or overlapping structures 216 may be provided from the controlled configuration of discrete lesions. For example, a series of one or more crossed-lesions 218 can be generated along a tissue region to facilitate various types of treatment methods.

The specific configurations of controlled thermal injury are selected to achieve the desired tissue and therapeutic effect. For example, any tissue effect can be realized, including but not limited to thermal and non-thermal streaming, cavitational, hydrodynamic, ablative, hemostatic, diathermic, and/or resonance-induced tissue effects. Additional embodiments useful for creating lesions may be found in US Patent Publication No. 20060116671 entitled “Method and System for Controlled Thermal Injury of Human Superficial Tissue” published Jun. 1, 2006 and incorporated by reference.

Now with reference to FIG. 5, treatment system 148, according to various embodiments, is illustrated. In various embodiments, treatment system comprises controller 144, display 146, ultrasound probe 105, and interface 142 for communication between ultrasound probe 105 and controller 144. Interface 142 can be a wired connection, a wireless connection or combinations thereof. Ultrasound probe 105 may be controlled and operated by controller 144, which also relays and processes images obtained by ultrasound probe 105 to display 146. In one embodiment, controller 144 is capable of coordination and control of the entire treatment process to achieve the desired therapeutic effect on muscle and connective tissue layer 112 within ROI 115. For example, in one embodiment, controller 144 may comprise power source components, sensing and monitoring components, cooling and coupling controls, and/or processing and control logic components. Controller 144 may be configured and optimized in a variety of ways with more or less subsystems and components to implement treatment system 148 for controlled targeting of a portion of muscle and connective tissue layer 112, and the embodiment in FIG. 4 is merely for illustration purposes.

For example, for power sourcing components, controller 144 may comprise one or more direct current (DC) power supplies capable of providing electrical energy for the entire controller 144, including power required by a transducer electronic amplifier/driver. A DC current or voltage sense device may also be provided to confirm the level of power entering amplifiers/drivers for safety and monitoring purposes.

In one embodiment, amplifiers/drivers may comprise multi-channel or single channel power amplifiers and/or drivers. In one embodiment for transducer array configurations, amplifiers/drivers may also be configured with a beamformer to facilitate array focusing. One beamformer may be electrically excited by an oscillator/digitally controlled waveform synthesizer with related switching logic.

Power sourcing components may also comprise various filtering configurations. For example, switchable harmonic filters and/or matching may be used at the output of amplifier/driver to increase the drive efficiency and effectiveness. Power detection components may also be included to confirm appropriate operation and calibration. For example, electric power and other energy detection components may be used to monitor the amount of power entering ultrasound probe 105.

Various sensing and monitoring components may also be implemented within controller 144. For example, in one embodiment, monitoring, sensing, and interface control components may be capable of operating with various motion detection systems implemented within ultrasound probe 105, to receive and process information such as acoustic or other spatial and temporal information from ROI 115. Sensing and monitoring components may also comprise various controls, interfacing, and switches and/or power detectors. Such sensing and monitoring components may facilitate open-loop and/or closed-loop feedback systems within treatment system 148. In one embodiment, sensing and monitoring components may further comprise a sensor that may be connected to an audio or visual alarm system to prevent overuse of system. In this exemplary embodiment, the sensor may be capable of sensing the amount of energy transferred to the skin, and/or the time that treatment system 148 has been actively emitting energy. When a certain time or temperature threshold has been reached, the alarm may sound an audible alarm, or cause a visual indicator to activate to alert the user that a threshold has been reached. This may prevent overuse of treatment system 148. In one embodiment, the sensor may be operatively connected to controller 144 and force controller 144, to stop emitting therapeutic ultrasound energy 120 from ultrasound probe 105.

Additionally, one controller 144 may further comprise a system processor and various digital control logic, such as one or more of microcontrollers, microprocessors, field-programmable gate arrays, computer boards, and associated components, including firmware and control software, which may be capable of interfacing with user controls and interfacing circuits as well as input/output circuits and systems for communications, displays, interfacing, storage, documentation, and other useful functions. System software may be capable of controlling all initialization, timing, level setting, monitoring, safety monitoring, and all other system functions required to accomplish user-defined treatment objectives. Further, various control switches may also be suitably configured to control operation.

With reference again to FIG. 5, one treatment system 148 also may comprise display 146 capable of providing images of ROI 115 in various embodiments where ultrasound energy may be emitted from ultrasound probe 105 in a manner for imaging. In one embodiment, display 146 is a computer monitor. Display 146 may be capable of enabling the user to facilitate localization of treatment area and surrounding structures, for example, identification of muscle and connective tissue layer 112. In an alternative exemplary embodiment, the user may know the location of the specific muscle and connective tissue layer 112 to be treated based at least in part upon prior experience or education and without display 146.

After localization, therapeutic ultrasound energy 120 is delivered at a depth, distribution, timing, and energy level to achieve the desired therapeutic effect at ROI 115 to treat injury. Before, during and/or after delivery of therapeutic ultrasound energy 120, monitoring of the treatment area and surrounding structures may be conducted to further plan and assess the results and/or provide feedback to controller 148, and to a system operator via display 146. In one embodiment, localization may be facilitated through ultrasound imaging that may be used to define the position of injury location and/or cartilage in ROI 115.

Feedback information may be generated or provided by any one or more acoustical sources, such as B-scan images, A-lines, Doppler or color flow images, surface acoustic wave devices, hydrophones, elasticity measurement, or shear wave based devices. In addition, optical sources can also be utilized, such as video and/or infrared cameras, laser Doppler imagers, optical coherence tomography imagers, and temperature sensors. Further, feedback information can also be provided by semiconductors, such as thermistors or solid state temperature sensors, by electronic and electromagnetic sensors, such as impedance and capacitance measurement devices and/or thermocouples, and by mechanical sensors, such as stiffness gages, strain gages or stress measurement sensors, or any suitably combination thereof. Moreover, various other switches, acoustic or other sensing mechanisms and methods may be employed to enable transducer 75 to be acoustically coupled to one or more ROI 115.

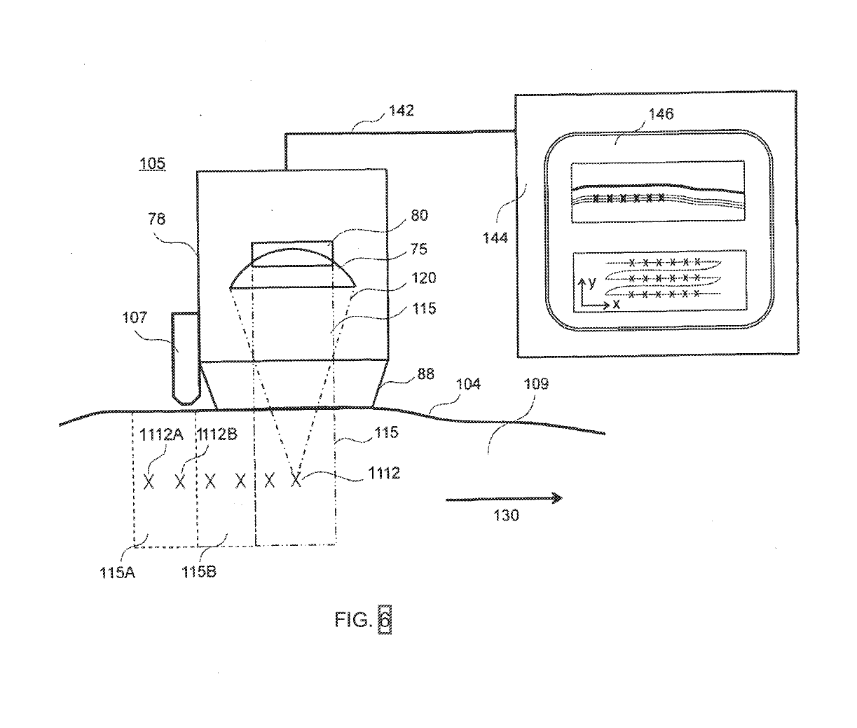

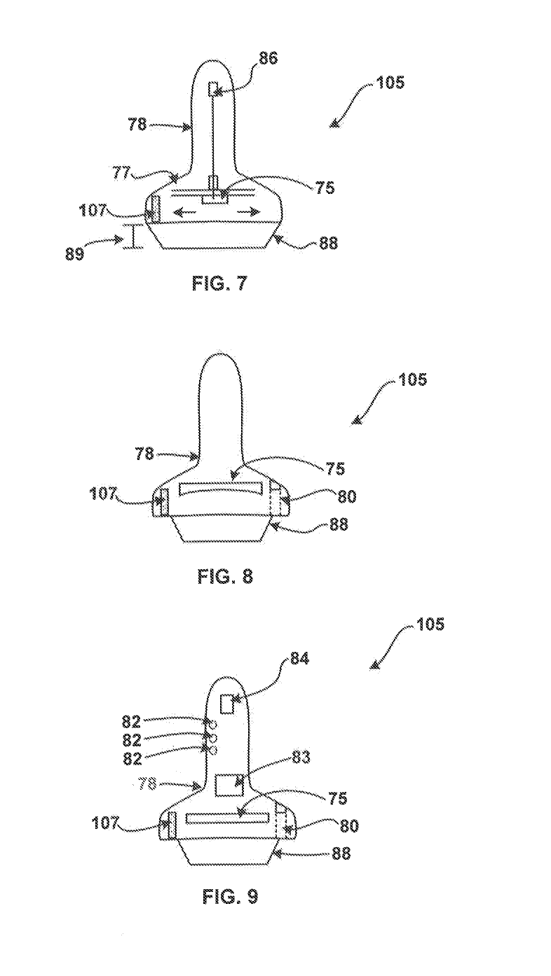

Moving to FIGS. 6 and 7, ultrasound probe 105 comprising a transducer and a motion mechanism, according to various embodiments, is illustrated. In various embodiments, ultrasound probe 105 comprises transducer 75. In some embodiments, ultrasound probe 105 comprises motion mechanism 77, which moves transducer 75 along a plane substantially parallel to skin surface 104. Motion mechanism 77 can be coupled to motor 86. Motion mechanism 77 can be controlled by controller 144. In some embodiments, ultrasound probe 105 comprises position sensor 107, as described herein.

In various embodiments, for example as illustrated in FIGS. 6-9, position sensor 107 can be integrated into ultrasound probe 105 or attached to ultrasound probe 105. In one embodiment, position sensor 107 is an optical sensor measuring 1-D, 2-D, or 3-D movement 130 of ultrasound probe 105 versus time while probe travels along skin surface 104. Such a position sensor may control ablation sequence 1112A, 1112B, . . . 1112n directly, by using position information in the treatment system to trigger ablation. In various embodiments, therapy can be triggered when the ultrasound probe 105 reaches a fixed or pre-determined range away from the last ablation zone 1112. Speed of motion can be used to control therapeutic ultrasound energy 108. For example, if the motion is too fast information can be provided to the user to slow down and/or energy can be dynamically adjusted within limits. Position information may also be used to suppress energy if crossing over the same spatial position, if desired. Such a position sensor 107 may also determine if ultrasound probe 105 is coupled to skin surface 104, to safely control energy delivery to subcutaneous tissue layer 109 and to provide information to users. Position sensor data acquisition can be synchronized with imaging sequence and monitoring sequence, to geo-tag and arrange the image frames 115A, 115B, . . . 115n and so on, in the correct spatial orientation to form an extended image, or likewise extended monitoring image, for display 146.

Extended position versus time data can be stored as tracking information, 123, and linked with the extended treatment sequence, 1112A, 1112B, . . . 1112n may be rendered as a graphical treatment map and rendered on display 146. Treatment map can be displayed as 2-D or multidimensional data, and can be real-time. In some embodiments, all extended images, extended monitoring images, treatment sequences, and treatment maps can be stored and played back as movies, images, or electronic records. Treatment map can be used to illustrate where treatment has occurred and/or to help the user fill-in untreated areas, especially if the user cannot see the treatment surface. In one embodiment, a projector can be used to overlay the treatment map atop the treatment surface, or the treatment map can be superimposed atop other visualizations of the treatment surface.

However, in various embodiments, ultrasound probe 105 comprises position sensor 107. Position sensor 107 can be integrated into ultrasound probe 105 or attached to ultrasound probe 105. In one embodiment, position sensor 107 is a motion sensor measuring movement of ultrasound probe 105. Such a motion sensor can calculate distance traveled along skin surface 104. Such a motion sensor may determine a speed of movement of ultrasound probe 105 along skin surface 104 and determine if the speed is accurate for treatment. For example if the speed is too fast, motion sensor can signal an indicator to slow the speed and/or can signal transducer to stop emitting therapeutic ultrasound energy 120.

In various embodiments, position sensor 107 comprises a visual element such as a camera or video capture device. In such embodiments, skin surface 104 can be geotagged. Features on the skin surface, such as, for example, a scar, a nipple, a belly button, a mole, an ankle, a knee cap, a hip bone, a mark, a tattoo, or combinations thereof and the like, may be geotagged using position sensor 107. A geotagged feature may be useful for treatment. A geotagged feature may be useful for setting parameters for treatment. A geotagged feature may be useful for determining progress or success of treatment. A geotagged feature may be useful to position ultrasound probe for a second treatment of injury location. A geotagged feature can be stored with other treatment parameters and/or treatment results.

In various embodiments, position sensor 107 can include a laser position sensor. For example, position sensor 107 can track position like a computer mouse that uses a laser sensor as opposed to an older version of a mouse with a roller ball. Position sensor 107 can communicate to a display to track a position of ultrasound probe 105, such as, for example, overlaid on an image of ROI 115, overlaid on an image of skin surface 104, as referenced to geotagged features, as reference to injury location, as referenced to a prior treatment, and combinations thereof. In one a treatment plan can include a movement pattern of ultrasound probe 105. Such a movement pattern can be displayed and the position sensor 1 07 can track a position of ultrasound probe 105 during treatment as compared to the movement pattern. Tracking ultrasound probe 105 with position sensor and comparing the tracked movement to a predetermined movement may be useful as a training tool. In one embodiment, laser position sensor can geotag a feature on skin surface 104.

In various embodiments, position sensor 107 may determine a distance 117 between pulses of therapeutic ultrasound energy 120 to create a plurality of lesions which are evenly space. As ultrasound probe 105 is moved in direction 130, position sensor 107 determines distance 117, regardless of a speed that ultrasound probe 105 is move, at which a pulse of therapeutic ultrasound energy 120 is to be emitted in to ROI 115.

Position sensor 107 may be located behind the transducer element, in front of the transducer element, or integrated into the transducer element. Ultrasound probe 105 may comprise more than one position sensor 107, such as, for example, a laser position sensor and a motion sensor, or a laser position sensor and a visual device, or a motion sensor and a visual device, or a laser position sensor, a motion sensor, and a visual device. Additional embodiments of position sensor 107 may be found in U.S. Pat. No. 7,142,905, entitled “Visual Imaging System for Ultrasonic Probe” issued Nov. 28, 2006, and U.S. Pat. No. 6,540,679, entitled “Visual Imaging System for Ultrasonic Probe” issued Apr. 1, 2003, both of which are incorporated by reference.

In some embodiments, transducer 75 is a single element operable for imaging and emitting therapeutic ultrasound energy 120, as described herein. In some embodiments, transducer 75 is a multi-element array operable for imaging and emitting therapeutic ultrasound energy 120, as described herein. However, in some embodiments, transducer 75 is operable for emitting therapeutic ultrasound energy 120 and is not operable for imaging, as described herein.

In various embodiments, transducer 75, motion mechanism 77, motor 87, optionally position sensor 107 can held within enclosure 78. In one embodiment, enclosure 78 is designed for comfort and control while used in an operator's hand. Enclosure 78 may also contain various electronics, EEPROM, interface connection, and/or ram for holding programs. In various embodiments, ultrasound probe 105 comprises tip 88. In some embodiments, tip 88 is gel and/or liquid filled. Tip can include EEPROM which is in communication with at least one of electronics in ultrasound probe 105 and controller 144. Data for EEPROM can be collected in controller 144 and connected to treatment data. In one embodiment, tip is disposable, and for example EEPROM determines if tip has been used and will not allow treatment to begin tip 88 that has been previously used. In some embodiments, tip 88 has height 89 which can control therapeutic ultrasound energy 120 depth into muscle and connective tissue layer 112. In some embodiments, a plurality of tips 88, each having a different height 89 may be used to direct therapeutic ultrasound energy 120 to a plurality of depths in muscle and connective tissue layer 112.

Transducer 75 may further comprise a functional surface, tip 88, or area at the end of the transducer 75 that modulates therapeutic ultrasound energy 120. This tip may enhance, magnify, or otherwise change therapeutic ultrasound energy 120 emitted from ultrasound probe 105. According to various embodiments, ultrasound probe 105 is coupled directly to cartilage, as opposed to skin surface 104, to treat cartilage. In some embodiments, ultrasound probe 105 can be integrated to or attached to a tool, such as, for example, an arthroscopic tool, laparoscopic tool, or an endoscopic tool that may be inserted into a patient's body with minimal invasiveness. In some embodiments, an arthroscopic tool can comprise probe 105 on a distal end. In some embodiments, probe 105 can be designed to be inserted into a body. In some embodiments, an arthroscopic probe can comprise a housing 78 on a distal end of the probe 105. In some embodiments, the housing 78 can contain an ultrasound transducer configured to focus a conformal distribution of ultrasound energy to ablate and fracture at least one of cartilage and surrounding tissue in an injury location; a position sensor configured to communicate a position of the housing and a speed of movement of the housing; a communication interface configured for wireless communication and in communication with the ultrasound transducer, and the position sensor; and a rechargeable power supply configured to supply power to the ultrasound transducer, the position sensor, and the communication interface.

Therapeutic ultrasound energy 120 from transducer 75 may be spatially and/or temporally controlled at least in part by changing the spatial parameters of transducer 75, such as the placement, distance, treatment depth and transducer 75 structure, as well as by changing the temporal parameters of transducer 75, such as the frequency, drive amplitude, and timing, with such control handled via controller 144. Such spatial and temporal parameters may also be monitored in open-loop and/or closed-loop feedback systems within treatment system 148. In various embodiments, ultrasound probe 105 comprises a transducer 75 capable of emitting therapeutic ultrasound energy 120 into ROI 115. This may heat ROI 115 at a specific depth to target muscle and connective tissue layer 112 causing that tissue to be ablated, micro-ablated, coagulated, incapacitated, partially incapacitated, rejuvenated, shortened, paralyzed, or removed.

A coupling gel may be used to couple ultrasound probe 105 to ROI 115. Therapeutic ultrasound energy 120 may be emitted in various energy fields in this exemplary embodiment. In this exemplary embodiment, the energy fields may be focused, defocused, and/or made substantially planar by transducer 75, to provide many different effects. Energy may be applied in a C-plane or C-scan. For example, in one exemplary embodiment, a generally substantially planar energy field may provide a heating and/or pretreatment effect, a focused energy field may provide a more concentrated source of heat or hypothermal effect, and a non-focused energy field may provide diffused heating effects. It should be noted that the term “nonfocused” as used throughout encompasses energy that is unfocused or defocused.

In various embodiments, transducer 75 may comprise one or more transducers elements for facilitating treatment. Transducer 75 may further comprise one or more transduction elements. Transduction element may comprise piezoelectrically active material, such as lead zirconate titanate (PZT), or other piezoelectrically active material such as, but not limited to, a piezoelectric ceramic, crystal, plastic, and/or composite materials, as well as lithium niobate, lead titanate, barium titanate, and/or lead metaniobate. In addition to, or instead of, a piezoelectrically active material. Transducer 75 may comprise any other materials configured for generating radiation and/or acoustical energy. Transducer 75 may also comprise one or more matching and/or backing layers configured along with the transduction element, such as being coupled to the piezoelectrically active material. Transducer 75 may also be configured with single or multiple damping elements along the transduction element.

In one embodiment, the thickness of the transduction element of transducer 75 may be configured to be uniform. That is, the transduction element may be configured to have a thickness that is generally substantially the same throughout. In another exemplary embodiment, the transduction element may also be configured with a variable thickness, and/or as a multiple damped device. For example, the transduction element of transducer 75 may be configured to have a first thickness selected to provide a center operating frequency of a lower range, for example from about 1 kHz to about 3 MHz, or from about 30 kHz to about 1 MHz, or from about 300 kHz to about 3 MHz, or about 500 kHz to about 1 MHz. The transduction element may also be configured with a second thickness selected to provide a center operating frequency of a higher range, for example from about 1 MHz to about 1 00 MHz, or from about 3 MHz to about 50 MHz, or from about 5 MHz to about 40 MHz, or from about 3 MHz to about 30 MHz, or any other frequency range described herein.

In yet another exemplary embodiment, transducer 75 may be configured as a single broadband transducer excited with two or more frequencies to provide an adequate output for raising the temperature within ROI 115 to the desired level. Transducer 75 may also be configured as two or more individual transducers, wherein each transducer 75 may comprise a transduction element. The thickness of the transduction elements may be configured to provide center-operating frequencies in a desired treatment range. For example, in one embodiment, transducer 75 may comprise a first transducer 75 configured with a first transduction element having a thickness corresponding to a center frequency range of about 1 kHz to about 3 MHz, or from about 30 kHz to about 1 MHz, or from about 300 kHz to about 3 MHz, or about 500 kHz to about 1 MHz and a second transducer 75 configured with a second transduction element having a thickness corresponding to a center frequency of about 1 MHz to about 100 MHz, or from about 3 MHz to about 50 MHz, or from about 5 MHz to about 40 MHz, or from about 3 MHz to about 30 MHz, or any other frequency range described herein.

Moreover, in some embodiments, any variety of mechanical lenses or variable focus lenses, e.g. liquid-filled lenses, may also be used to focus and or defocus the energy field. For example, transducer 75 may also be configured with an electronic focusing array in combination with one or more transduction elements to facilitate increased flexibility in treating ROI 115. Array may be configured in a manner similar to transducer 75. That is, array may be configured as an array of electronic apertures that may be operated by a variety of phases via variable electronic time delays. Accordingly, the electronic apertures of array may be manipulated, driven, used, configured to produce and/or deliver energy in a manner corresponding to the phase variation caused. by the electronic time delay. For example, these phase variations may be used to deliver defocused beams, planar beams, and/or focused beams, each of which may be used in combination to achieve different physiological effects in ROI 115. Transduction elements may be configured to be concave, convex, and/or planar. For example, transduction elements can be configured to be concave in order to provide focused energy for treatment of ROI 115. In another exemplary embodiment, transduction elements may be configured to be substantially flat in order to provide substantially uniform energy to ROI 115. In addition, transduction elements may be configured to be any combination of concave, convex, and/or substantially flat structures. For example, a first transduction element may be configured to be concave, while a second transduction element may be configured to be substantially flat.

Moreover, transduction element can be any distance from the patient's skin. In that regard, it can be far away from the skin surface 104 disposed within a long transducer 75 or it can be just a few millimeters from skin surface 104. In certain exemplary embodiments, positioning the transduction element closer to skin surface 104 is better for emitting ultrasound at high frequencies. Moreover, both two and three dimensional arrays of transduction elements can be used in various embodiments.

In some embodiments, transducer 75 may also be configured as an annular array to provide planar, focused and/or defocused acoustical energy. For example, in one embodiment, an annular array may comprise a plurality of rings. Rings may be mechanically and electrically isolated into a set of individual elements, and may create planar, focused, or defocused waves. For example, such waves can be centered on-axis, such as by methods of adjusting corresponding phase delays. An electronic focus may be moved along various depth positions in ROI 115, and may enable variable strength or beam tightness, while an electronic defocus may have varying amounts of defocusing. In one embodiment, a lens and/or convex or concave shaped annular array may also be provided to aid focusing or defocusing such that any time differential delays can be reduced. Movement of annular array in one, two or three dimensions, or along any path, such as through use of probes, motion mechanisms, any conventional robotic arm mechanisms, and the like may be implemented to scan and/or treat a volume or any corresponding space within ROI 115.

In some embodiments, a cooling/coupling control system may be provided, and may be capable of removing waste heat from ultrasound probe 105. Furthermore the cooling/coupling control system may be capable of providing a controlled temperature at skin surface 104 and deeper into tissue, and/or provide acoustic coupling from ultrasound probe 105 to ROI 115. Such cooling/coupling control systems can also be capable of operating in both open-loop and/or closed-loop feedback arrangements with various coupling and feedback components.

With reference to FIG. 8, an ultrasound probe comprising a transducer, according to various embodiments, is illustrated. In various embodiments, ultrasound probe 105 comprises transducer 75. In some embodiments, ultrasound probe 105 comprises imaging transducer 80. In some embodiments, ultrasound probe 105 comprises position sensor 107, as described herein. In some embodiments, transducer 75 is operable for emitting therapeutic ultrasound energy 120 and imaging transducer 80 is operable for imaging, as described herein.

In various embodiments, ultrasound probe 105 can comprise a tissue contact sensor. In one embodiment, tissue contact sensor communicates whether ultrasound probe 105 is coupled to the ROI 115. The tissue contact sensor may measure a capacity of a skin surface 104 above the ROI 115 and communicate a difference between the capacity of the contact to the skin surface 104 and the capacity of air. In one embodiment, the tissue contact sensor is initiated or turned on by pressing ultrasound probe against skin surface 104.

In various embodiments, transducer 75, imaging transducer 80, and optionally position sensor 107, can be held within enclosure 78. In one embodiment, enclosure 78 is designed for comfort and control while used in an operator's hand. Enclosure 78 may also contain various electronics, EEPROM, interface connection, and/or ram for holding programs. In various embodiments, ultrasound probe 105 comprises tip 88. In some embodiments, tip 88 is gel and/or liquid filled. Tip can include EEPROM which is in communication with at least one of electronics in ultrasound probe 105 and controller 144. Data for EEPROM can be collected in controller 144 and connected to treatment data. In one embodiment, tip is disposable, and for example EEPROM determines if tip has been used and will not allow treatment to begin tip 88 that has been previously used. In some embodiments, tip 88 has height 89 which can control therapeutic ultrasound energy 120 depth into muscle and connective tissue layer 112. In some embodiments, a plurality of tips 88, each having a different height 89 may be used to direct therapeutic ultrasound energy 120 to a plurality of depths in muscle and connective tissue layer 112.

With reference to FIG. 9, a hand held ultrasound probe, according to various embodiments, is illustrated. In various embodiments, ultrasound transducer 105 comprises transducer 75, as described herein, and may be controlled and operated by a hand-held format control system. An external battery charger can be used with rechargeable-type batteries 84 or the batteries 84 can be single-use disposable types, such as M-sized cells. Power converters produce voltages for powering a driver/feedback circuit with tuning network driving transducer 75. Ultrasound probe 105 is coupled to skin surface 104 via one or more tips 88, which can be composed of at least one of a solid media, semi-solid e.g. gelatinous media, and/or liquid media equivalent to an acoustic coupling agent (contained within a housing). Tip 88 is coupled to skin surface 104 with an acoustic coupling agent. In addition, a microcontroller and timing circuits with associated software and algorithms provide control and user interfacing via a display or LED-type indicators 83, and other input/output controls 82, such as switches and audio devices. A storage element, such as an Electrically Erasable Programmable Read-Only Memory (“EEPROM”), secure EEPROM, tamper-proof EEPROM, or similar device can hold calibration and usage data. A motion mechanism with feedback can be controlled to scan the transducer 75 in a linear pattern or a two-dimensional pattern or over a varied depth. Other feedback controls comprise capacitive, acoustic, or other coupling detection means, limiting controls, and thermal sensor. EEPROM can be coupled with at least one of tip 88, transducer 75, thermal sensor, coupling detector, and tuning network. Data for EEPROM can be collected in controller 144 and connected to treatment data.

Ultrasound probe 105 can comprise tip 88 that can be disposed of after contacting a patient. In one embodiment, tip is disposable, and for example EEPROM determines if tip has been used and will not allow treatment to begin if tip 88 has been previously used. In some embodiments, ultrasound probe 105 comprises imaging transducer 80. In some embodiments, ultrasound probe 105 comprises position sensor 107, as described herein. In some embodiments, transducer 75 is operable for emitting therapeutic ultrasound energy 120 and imaging transducer 80 is operable for imaging, as described herein.

In various embodiments, ultrasound probe 105 comprises transducer 75. In some embodiments, ultrasound probe 105 comprises position sensor 107, as described herein. In some embodiments, transducer 75 is a single element operable for imaging and emitting therapeutic ultrasound energy 120, as described herein. In some embodiments, transducer 75 is a multi-element array operable for imaging and emitting therapeutic ultrasound energy 120, as described herein. However, in some embodiments, transducer 75 is operable for emitting therapeutic ultrasound energy 120 and is not operable for imaging, as described herein.

In various embodiments, transducer 75, and optionally position sensor 107 can held within enclosure 78. In one embodiment, enclosure 78 is designed for comfort and control while used in an operator's hand. Enclosure 78 may also contain various electronics, EEPROM, interface connection, motion mechanisms, and/or ram for holding programs.

In various embodiments, ultrasound probe 105 can be in communication with wireless device via wireless interface. Typically, wireless device has display and a user interface such as, for example, a keyboard. Examples of wireless device can include but are not limited to: personal data assistants (“PDA”), cell phone, iPhone, iPad, computer, laptop, netbook, or any other such device now known or developed in the future. Examples of wireless interface include but are not limited to any wireless interface described herein and any such wireless interface now known or developed in the future. Accordingly, ultrasound probe 105 comprises any hardware, such as, for example, electronics, antenna, and the like, as well as, any software that may be used to communicate via wireless interface.

In various embodiments, wireless device can display an image generated by handheld probe 105. In various embodiments, wireless device can control handheld ultrasound probe 105. In various embodiments, wireless device can store data generated by handheld ultrasound probe 105.

Therapeutic ultrasound energy 120 from transducer 75 may be spatially and/or temporally controlled at least in part by changing the spatial parameters of transducer 75, such as the placement, distance, treatment depth and transducer 75 structure, as well as by changing the temporal parameters of transducer 75, such as the frequency, drive amplitude, and timing, with such control handled via controller in hand-held assembly of ultrasound probe 105. In various embodiments, ultrasound probe 105 comprises a transducer 75 capable of emitting therapeutic ultrasound energy 120 into ROI 115. This may heat ROI 115 at a specific depth to target muscle and connective tissue layer 112 causing that tissue to be ablated, micro-ablated, coagulated, incapacitated, partially incapacitated, rejuvenated, shortened, paralyzed, or removed.

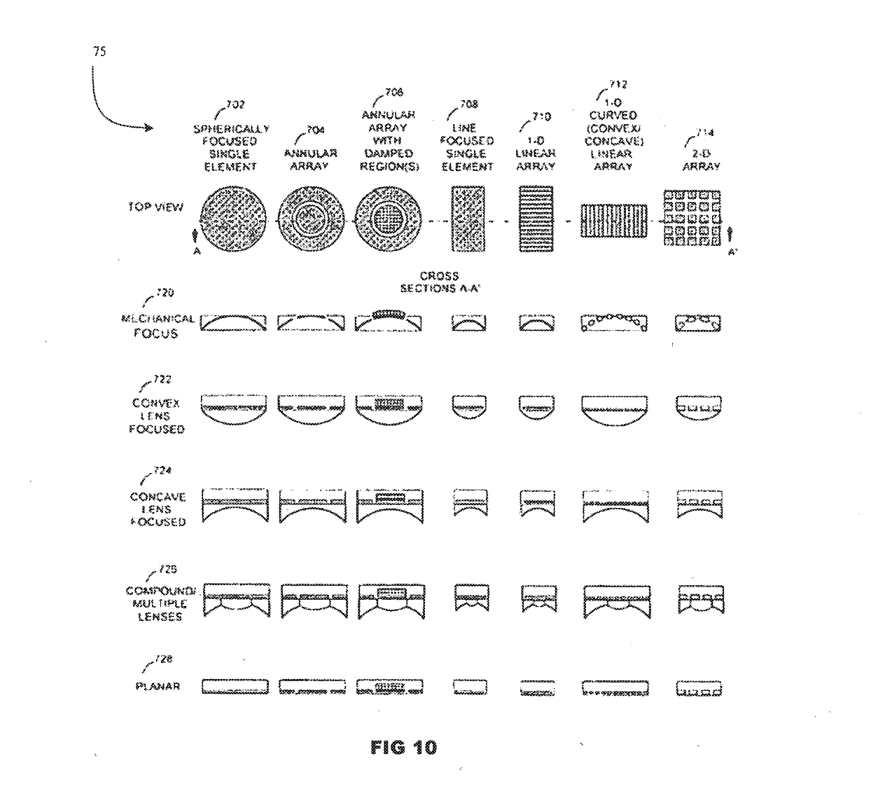

Referring to FIG. 10, a plurality of exemplary transducer configurations, according to various embodiments, is illustrated. In some embodiments, transducer 75 can be configured to comprise a spherically focused single element 36, annular/multi-element 38, annular with imaging region(s) 40, line-focused single element 42, 1-D linear array 44, 1-D curved (convex/concave) linear array 46, or 2-D array 48. With further reference to FIG. 10, any of the previous described configuration of transducer 75 can be coupled to one of mechanical focus 50, convex lens focus 52, concave lens focus 54, compound/multiple lens focused 56, or planar array form 58, and combinations thereof. Such transducer 75 configurations individually or coupled to a focusing element can achieve focused, unfocused, or defocused sound fields for at least one of imaging and therapy.

In some embodiments, damaged cartilage 140 can be from a joint injury, avascular necrosis, osteoarthritis, and rheumatoid arthritis. In one embodiment, the damaged cartilage 140 can be torn cartilage 140. In one embodiment, the damaged cartilage 140 can be a torn meniscus. In one embodiment, the damaged cartilage 140 is a partial tear in cartilage 140. In some embodiments, the damaged cartilage 140 is not in a joint, but rather in a nose, an ear, in a face, or any other such location in a body. In various embodiments, the damaged cartilage 140 is in a joint.

The meniscus is a C-shaped piece of cartilage 140. Cartilage 140 is found in certain joints and forms a buffer between the bones to protect the joint. The meniscus serves as a shock-absorption system, assists in lubricating the joint, and limits the ability to flex and extend the joint. Meniscal tears are most commonly caused by twisting or over-flexing the joint. The majority of the meniscus has a very poor blood supply, and does not heal.

The most commonly performed surgical procedures on the knee include a meniscectomy, meniscal repair, and ligament reconstruction. The traditional method of surgery for a torn meniscus involves admission to a hospital for several days, one or more surgical incisions that may average several inches, several weeks on crutches, and up to several months to completely rehabilitate the knee. Techniques of meniscus repair include using allhroscopically placed tacks or suturing the torn edges. Both procedures function by reapproximating the torn edges of the meniscus to allow them to heal in their proper place and not get caught in the knee.

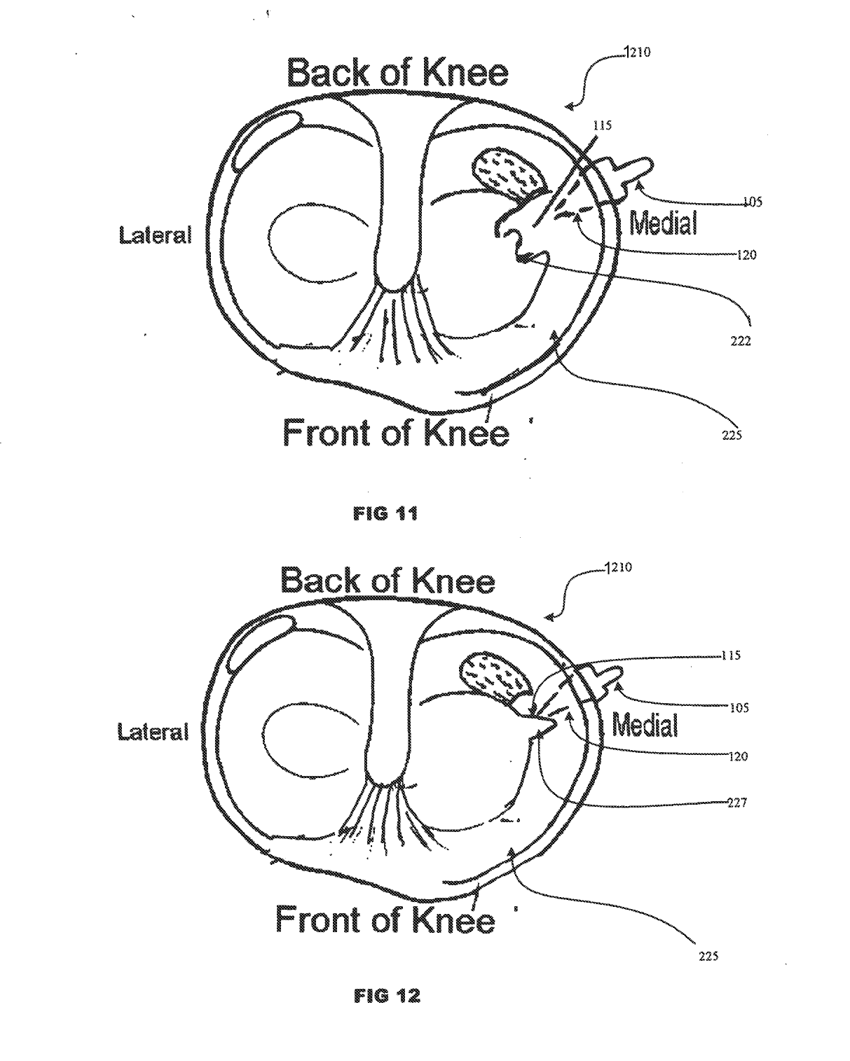

With reference to FIG. 11, according to various embodiments, methods of treating a torn meniscus 222 are provided. Such a method can include targeting the torn meniscus 222 in ROI 115, directing therapeutic ultrasound energy 120 to the torn meniscus 222 ablating at least a portion of the torn meniscus 222, and improving the torn meniscus 222. The method can include coupling ultrasound probe 105 to ROI 115. The method can include focusing therapeutic ultrasound energy 120 to create a conformal region of elevated temperature in a portion of the torn meniscus 222. In some embodiments, can include focusing therapeutic ultrasound energy 120 to create a lesion in a portion of the torn meniscus 222. The method can include creating a plurality of lesions in the torn meniscus 222. The method can include creating the plurality of lesion in a pattern, such as, a linear pattern, a 2-D pattern, or a 3-D pattern, and combinations thereof. The method further comprising measuring a distance on skin surface 104 and then directing therapeutic ultrasound energy 120 to the torn meniscus 222. The method can also include imaging the torn meniscus 222. The method can also include imaging the torn meniscus 222 after the ablating at least a portion of the torn meniscus 222. The method can include comparing a measurement of the torn meniscus 222 before and after the ablating step. The method can include directing acoustical pressure or cavitation to the torn meniscus 222 after the ablating step further improving torn meniscus 222. The method can include increasing blood perfusion to ROI 115. The method can include cutting the torn meniscus 222 from meniscus 225 with therapeutic ultrasound energy 120. The method can include imaging the torn meniscus 222 and cutting the torn meniscus 222 with therapeutic ultrasound energy 120 simultaneously. The method can include smoothing the meniscus 225 with therapeutic ultrasound energy 120. The method can include regenerating cartilage 140 in meniscus 225, according to methods described herein. The method can include administering a medicant to ROI 115. The method can be applied to cartilage in any joint on the body. The method can repair the function of a knee 1210. The method can also include any of the steps of method 100. According to various embodiments, ultrasound probe 105 is coupled directly to cartilage, as opposed to skin surface 104, to image, treat, and monitor cartilage. In some embodiments, ultrasound probe 105 can be integrated to or attached to a tool, such as, for example, an arthroscopic tool, laparoscopic tool, or an endoscopic tool that may be inserted into a patient's body with minimal invasiveness.

With reference to FIG. 12, according to various embodiments, methods of treating a torn meniscus 227 are provided. Such a method can include targeting the torn meniscus 227 in ROI 115, directing therapeutic ultrasound energy 120 to the torn meniscus 227 ablating at least a portion of the torn meniscus 227, and improving the torn meniscus 227. The method can include coupling ultrasound probe 105 to ROI 115. The method can include focusing therapeutic ultrasound energy 120 to create a conformal region of elevated temperature in a portion of the torn meniscus 222. In some embodiments, can include focusing therapeutic ultrasound energy 120 to create a lesion in a portion of the torn meniscus 227. The method can include creating a plurality of lesions in the torn meniscus 227. The method can include creating the plurality of lesion in a pattern, such as, a linear pattern, a 2-D pattern, or a 3-D. pattern, and combinations thereof. The method further comprising measuring a distance on skin surface 104 and then directing therapeutic ultrasound energy 120 to the torn meniscus 227. The method can also include imaging the torn meniscus 227. The method can also include imaging the torn meniscus 227 after the ablating at least a portion of the torn meniscus 227. The method can include comparing a measurement of the torn meniscus 227 before and after the ablating step. The method can include directing acoustical pressure or cavitation to the torn meniscus 227 after the ablating step further improving torn meniscus 227. The method can include increasing blood perfusion to ROI 115. The method can include welding together the torn meniscus 227 with therapeutic ultrasound energy 120. The method can include imaging the torn meniscus 227 and welding together the torn meniscus 227 with therapeutic ultrasound energy 120 simultaneously. The method can include smoothing the meniscus 227 with therapeutic ultrasound energy 120. The method can include regenerating cartilage 140 in meniscus 227, according to methods described herein. The method can include administering a medicant to ROI 115. The method can be applied to cartilage 140 in any joint on the body. The method can also include any of the steps of method 100.

According to various embodiments, methods of treating a damaged cartilage 140 are provided. Such a method can include targeting the damaged cartilage 140 in ROI 115, directing therapeutic ultrasound energy 120 to the damaged cartilage 140 ablating at least a portion of the damaged cartilage 140, and improving the damaged cartilage 140. The method can include coupling ultrasound probe 105 to ROI 115. The method can include focusing therapeutic ultrasound energy 120 to create a conformal region of elevated temperature in a portion of the torn meniscus 222. In some embodiments, can include focusing therapeutic ultrasound energy 120 to create a lesion in a portion of the damaged cartilage 140. The method can include creating a plurality of lesions in the damaged cartilage 140. The method can include creating the plurality of lesion in a pattern, such as, a linear pattern, a 2-D pattern, or a 3-D pattern, and combinations thereof. The method further comprising measuring a distance on skin surface 104 and then directing therapeutic ultrasound energy 120 to the damaged cartilage 140. The method can also include imaging the damaged cartilage 140. The method can also include imaging the damaged cartilage 140 after the ablating at least a portion of the damaged cartilage 140. The method can include comparing a measurement of the damaged cartilage 140 before and after the ablating step. The method can include directing acoustical pressure or cavitation to the damaged cartilage 140 after the ablating step further improving damaged cartilage 140. The method can include increasing blood perfusion to ROI 115. The method can include welding together the damaged cartilage 140 with therapeutic ultrasound energy 120. The method can include imaging the damaged cartilage 140 and welding together the damaged cartilage 140 with therapeutic ultrasound energy 120 simultaneously. The method can include cutting the damaged cartilage 140 and removing it from the joint with therapeutic ultrasound energy 120. The method can include smoothing the cartilage 140 with therapeutic ultrasound energy 120. The method can include regenerating cartilage 140, according to methods described herein. The method can include administering a medicant to ROI 115. The method can be applied to cartilage 140 in any joint on the body. The method can also include any of the steps of method 100.

Various embodiments include methods for treating cartilage 140. The method can include applying therapeutic ultrasound energy 120 to ROI 115, which comprises any area of a body that comprises cartilage 140. For example, ROI 115 can include locations in the head, such as, nose, ears, soft palate, or joint sockets, such as, the knee, elbow, shoulders, hips, or the spine, or any other area of the body that comprises cartilage. For example, ROI 115 can include locations between the joints that contain cartilage such as the elbows, knees, shoulders, and any other joint. The therapeutic ultrasound energy 120 is applied to ROI 115 until a specific bio-effect is achieved, such as, for example, through cutting, reabsorbing or manipulating the cartilage. Certain exemplary bio-effects achieved by cutting, reabsorbing or manipulating the cartilage can comprise, but are not limited to, incapacitating, partially incapacitating, rejuvenating, ablating, micro-ablating, modifying, shortening, coagulating, paralyzing, or causing the cartilage to be reabsorbed into the body. As used throughout, the term “ablate” means to destroy or coagulate tissue at ROI 115. The term “micro-ablate” means to ablate on a smaller scale. Upon the completion of bio-effects, cartilage is treated and a clinical outcome, such as, for example, repair to a meniscus, or regeneration of cartilage in a joint. In various embodiments, methods are provided for cartilage 140 regeneration. Removing a portion of cartilage 140 from a patient will initiate cartilage regeneration in that ROI 115. In this regard, traditionally invasive procedures that effectuate cartilage 140 regeneration can be performed non-invasively using energy such as therapeutic ultrasound energy 120. In some embodiments, therapeutic ultrasound energy 120 is applied at ablative levels at ROI 115 to remove a portion of cartilage 140. Removing a portion of cartilage 140 enables cartilage regeneration to occur. In some embodiments, non-invasive micro-fracture surgery using therapeutic ultrasound energy 120 can be performed to encourage cartilage 140 regeneration.

In various embodiments, during micro-fracture surgery, therapeutic ultrasound energy 120 is applied at ablative levels to target cartilage or other subcutaneous tissues near cartilage 140 in the knee joint. Applying therapeutic ultrasound energy 120 at ablative levels near the knee joint causes one or more fractures in cartilage 140 or other subcutaneous tissue such as bones. When bones or other subcutaneous tissues are targeted, sufficient therapeutic ultrasound energy 120 is applied to ablate those tissues. These fractures result in cartilage 140 re-growing in the place of the ablated subcutaneous tissues and a non-invasive micro-fracture surgery is performed.

In various embodiments, cartilage 140 between the joints is treated with method 100. In this regard, swollen or otherwise injured cartilage 140 responsible for osteoarthritis, rheumatoid arthritis, and juvenile rheumatoid arthritis can be treated with method 100. For example, ROI 115 may be along a patient's knees to treat cartilage that serves as a cushion in a patient's knee socket. Alternatively, ROI 115 can be disposed on a patient's shoulder area to treat cartilage 140 disposed on the shoulder joint. In some embodiments, therapeutic ultrasound energy 120 may not be applied at ablative levels but at levels that produce enough heat at ROI 115 to reduce swelling and the size of cartilage 140 within these joints.

In various embodiments, cartilage between bones in the spine is treated by method 100. In one embodiment, methods described herein may be used to treat degenerative disc disease. Still further, methods described herein may be used to treat a disc in the spine. For example, methods described herein may be used to weld a tear in a disc together. In another example, methods and systems described herein may be used to perform a intervertebral disc annuloplasty, whereby a disc is heated to over 80° C. or to over 90° C. to seal a disc. In one embodiment, a method of treating a disc includes a minimally invasive procedure to couple ultrasound probe 105 to disc to be treated.

According to various embodiments, ultrasound probe 105 is coupled directly to cartilage 140, as opposed to skin surface 104, to at least one of image and treat cartilage. In some embodiments, ultrasound probe 105 can be integrated to or attached to a tool, such as, for example, an arthroscopic tool, laparoscopic tool, or an endoscopic tool that may be inserted into a patient's body with minimal invasiveness. Any steps of a minimally invasive procedure, such as arthroscopy, laparoscopy, endoscopy, and the like may be incorporated with any method described herein, including method 100.

The following patents and patent applications are incorporated by reference: US Patent Application Publication No. 20050256406, entitled “Method and System for Controlled Scanning, Imaging, and/or Therapy” published Nov. 17, 2005; US Patent Application Publication No. 20060058664, entitled “System and Method for Variable Depth Ultrasound Treatment” published Mar. 16, 2006; US Patent Application Publication No. 20060084891, entitled Method and System for Ultra-High Frequency Ultrasound Treatment” published Apr. 20, 2006; U.S. Pat. No. 7,530,958, entitled “Method and System for Combined Ultrasound Treatment” issued. May 12, 2009; US Patent Application Publication No. 2008071255, entitled “Method and System for Treating Muscle, Tendon, Ligament, and Cartilage Tissue” published Mar. 20, 2008; U.S. Pat. No. 6,623,430, entitled “Method and Apparatus for Safely Delivering Medicants to a Region of Tissue Using Imaging, Therapy, and Temperature Monitoring Ultrasonic System, issued Sep. 23, 2003; U.S. Pat. No. 7,571,336, entitled” Method and System for Enhancing Safety with Medical Peripheral Device by Monitoring if Host Computer is AC Powered” issued Aug. 4, 2009; and US Patent Application Publication No. 20080281255, entitled “Methods and Systems for Modulating Medicants Using Acoustic Energy” published Nov. 13, 2008.

It is believed that the disclosure set forth above encompasses at least one distinct invention with independent utility. While the invention has been disclosed in the exemplary forms, the specific embodiments thereof as disclosed and illustrated herein are not to be considered in a limiting sense as numerous variations are possible. The subject matter of the inventions includes all novel and non-obvious combinations and sub combinations of the various elements, features, functions and/or properties disclosed herein. Various embodiments and the examples described herein are exemplary and not intended to be limiting in describing the full scope of compositions and methods of this invention. Equivalent changes, modifications and variations of various embodiments, materials, compositions and methods may be made within the scope of the present invention, with substantially similar results.

The following description is in no way intended to limit the various embodiments, their application, or uses. As used herein, the phrase “at least one of A, B, and C” should be construed to mean a logical (A or B or C), using a non-exclusive logical or. As used herein, the phrase “A, B and/or C” should be construed to mean (A, B, and C) or alternatively (A or B or C), using a non-exclusive logical or. It should be understood that steps within a method may be executed in different order without altering the principles of the present disclosure.

The drawings described herein are for illustrative purposes only of selected embodiments and not all possible implementations, and are not intended to limit the scope of any of the various embodiments disclosed herein or any equivalents thereof It is understood that the drawings are not drawn to scale. For purposes of clarity, the same reference numbers will be used in the drawings to identify similar elements.

The various embodiments may be described herein in terms of various functional components and processing steps. It should be appreciated that such components and steps may be realized by any number of hardware components configured to perform the specified functions. For example, various embodiments may employ various medical treatment devices, visual imaging and display devices, input terminals and the like, which may carry out a variety of functions under the control of one or more control systems or other control devices. In addition, the embodiments may be practiced in any number of medical contexts and that the various embodiments relating to a method and system for acoustic tissue treatment as described herein are merely indicative of examples of applications. For example, the principles, features and methods discussed may be applied to any medical application. Further, various aspects of the various embodiments may be suitably applied to cosmetic applications. Moreover, some of the embodiments may be applied to cosmetic enhancement of skin and/or various subcutaneous tissue layers.

According to various embodiments, methods and systems useful for treating injuries to joints are provided herein. In some embodiments, methods and systems useful for permanent relief of pain in joints are provided herein. Various embodiments provide for combining therapeutic ultrasound energy directed to a joint with a medicant injected into the joint.

According to various embodiments, methods and systems useful for treating joint injuries are provided herein. The methods and systems provided herein can be noninvasive, for example, no cutting or injecting into the skin is required. Treating an injury to a joint using the methods and systems provided herein minimize recover time and may in some cases eliminate downtime for recovery. Further treating an injury to a joint using the methods and systems provided herein minimize discomfort to a patient having such a procedure.

Various embodiments described herein, provide a method for treating an injury in a joint of a body, In some embodiments the method comprises targeting a region of interest comprising the injury in the joint and tissue surrounding the joint and imaging the injury in the region of interest In addition, the method can comprise delivering ultrasound energy to the joint, creating a conformal region of elevated temperature in the joint, and initiating at least one thermally induced biological effect in the joint.

In some embodiments, the method can further comprise delivering a medicant to the joint and optionally can comprise activating the medicant in the joint. In some embodiments, the method can further comprise applying mechanical ultrasound energy to region of interest and delivering the medicant to the tissue surrounding the injury. In some embodiments, the delivering the medicant to the tissue surrounding the injury can minimize formation of scar tissue in the surrounding tissue.

In some embodiments, the method can further comprise stimulating a change to at least one of concentration and an activity of at least one of an inflammatory mediator and a growth factor in the joint. In some embodiments, the thermally induced biological effect is at least one of coagulation, increased perfusion, reduction of inflammation, generation of heat shock proteins, and initiation of healing cascade.

In some embodiments, the method can further comprise further comprising creating a lesion in the tissue in the joint and stimulating a wound healing cascade in the region of interest In some embodiments, the method can further comprise directing a second energy into the region of interest and creating a second therapeutic effect in the joint with the second energy. In some embodiments, the second energy is one of radiofrequency energy, photon-based energy, plasma-based energy, magnetic resonance energy, microwave energy, and mechanical energy. In some embodiments, the second energy is a second ultrasound emission at a different frequency. In some embodiments, the second therapeutic effect in the region of interest is one of is at least one of coagulation, increased perfusion, reduction of inflammation, generation of heat shock proteins, and initiation of healing cascade.

Various embodiments provide methods of treating an injury in a joint. In some embodiments, the method can comprise targeting injured fibrous soft tissue located in at least one of at and proximate to an injury location comprising a portion of a joint and directing therapeutic ultrasound energy to the injured fibrous soft tissue. In some embodiments, the method can comprise creating a conformal region of elevated temperature in the injured fibrous soft tissue, and creating at least one thermally induced biological effect in the injured fibrous soft tissue.

In some embodiments, the thermally induced biological effect is at least one of coagulation, increased perfusion, reduction of inflammation, generation of heat shock proteins, and initiation of healing cascade. In some embodiments, the method can further comprise imaging the injured soft fibrous tissue. In some embodiments, the method can further comprise driving a medicant into the injured soft fibrous tissue. In some embodiments, the method can further comprise activating the medicant with the therapeutic ultrasound energy. In some embodiments, the method can further comprise peaking inflammation in the injury location and initiating a coagulation cascade in at least a portion of the joint.

In some embodiments, the method can further comprise welding a portion of the injured fibrous soft tissue with the conformal ultrasound energy and repairing a tear in the portion of the joint. In some embodiments, the method can further comprise stimulating collagen growth in a portion of the joint with the conformal ultrasound energy. In some embodiments, the method can further comprise creating a plurality of lesions in a portion of a tendon of the joint; scoring a portion of the tendon; releasing strain in the tendon; and stimulating healing in the tendon. In some embodiments, the method can further comprise sparing intervening tissue between the injury in the joint and a skin surface above the region of interest.

Various embodiments provide a method of treating scar tissue in a joint. In some embodiments, the method can comprise targeting scar tissue in a joint; directing mechanical ultrasound energy to the scar tissue in the joint; and breaking up the scar tissue. In some embodiments, the method can further comprise directing ablative ultrasound energy to the joint: triggering inflammation in the joint with the ablative ultrasound energy; peaking inflammation in the joint; and accelerating healing in the joint.

In some embodiments, the method can further comprise shrinking at least a portion of the scar tissue in the joint. In some embodiments, the method can further comprise imaging the scar tissue in the joint. In some embodiments, the method can further comprise initiating a coagulation cascade in at least a portion of the joint. In some embodiments, the method can further comprise stimulating a change to at least one of concentration and an activity of at least one of an inflammatory mediator and a growth factor.

In some embodiments, the method can further comprise initiating a thermally induced biological effect in the joint. In some embodiments, the thermally induced biological effect is at least one of coagulation, increased perfusion, reduction of inflammation, generation of heat shock proteins, and initiation of healing cascade.

In some embodiments, the method can further comprise delivering a medicant to the joint and optionally activating the medicant in the joint. In some embodiments, the medicant reduces at least one of inflammation in the joint and pain in the joint. In some embodiments, the medicant reduces scarring in the joint. In some embodiments, the method can further comprise shrinking the scar tissue in the joint.

Various embodiments provide a method of providing pain relief in a joint. In some embodiments, the method can comprise identifying a location of pain in a joint; imaging the location in the joint; and identifying a nerve ending responsible for the pain in the joint. In some embodiments, the method can further comprise focusing ultrasound energy onto the nerve ending responsible for the pain in the joint; ablating the nerve ending with the ultrasound energy; disabling function of the nerve ending; and eliminating the pain in the joint.

In some embodiments, the method can further comprise directing ablative ultrasound energy to the joint; triggering inflammation in the joint with the ablative ultrasound energy; peaking inflammation in the joint; and accelerating healing in the joint.

In some embodiments, the method can further comprise delivering a medicant to the nerve ending. In some embodiments, the medicant is BoTox and the medicant is operable to disable function of the nerve ending. In some embodiments, the medicant is operable to stimulate healing in the joint. In some embodiments, the eliminating the pain in the joint is permanent. In some embodiments, the nerve is a sensory nerve and is not a nerve that controls motor function.

Various embodiments provide methods for treating a frozen joint. In one embodiment, the method can treat a frozen shoulder. Frozen shoulder, medically referred to as adhesive capsulitis, is a disorder in which the shoulder capsule, the connective tissue surrounding the glenohumeral joint of the shoulder, becomes inflamed and stiff: greatly restricting motion and causing chronic pain.

Such a method of treating a frozen shoulder can include targeting inflamed tissue near or at a portion of a capsule in the shoulder in ROI, directing therapeutic ultrasound energy to the inflamed tissue near or at a portion of a capsule in the shoulder, ablating at least a portion of the inflamed tissue near or at a portion of a capsule in the shoulder, and improving the inflamed tissue near or at a portion of a capsule in the shoulder. The method can include coupling ultrasound probe to ROI. The method can include focusing therapeutic ultrasound energy to create a lesion in a portion of the inflamed tissue near or at a portion of a capsule in the shoulder. The method can include creating a plurality of lesions in the inflamed tissue near or at a portion of a capsule in the shoulder. The method can include creating the plurality of lesion in a pattern, such as, a linear pattern, a 2-D pattern, or a 3-D pattern, and combinations thereof. The method further comprising measuring a distance on skin surface and then directing therapeutic ultrasound energy to the inflamed tissue near or at a portion of a capsule in the shoulder, The method can also include imaging an inflamed portion of a portion of a capsule in the shoulder. The method can also include imaging the inflamed tissue near or at a portion of a capsule in the shoulder after the ablating at least a portion of the inflamed tissue near or at a portion of a capsule in the shoulder. The method can include comparing a measurement of the inflamed tissue near or at a portion of a capsule in the shoulder before and after the ablating step. The method can include directing acoustical pressure or cavitation to the inflamed tissue near or at a portion of a capsule in the shoulder after the ablating step further improving the inflamed tissue near or at a portion of a capsule in the shoulder. The method can include increasing blood perfusion to the ROI.

According to various embodiments, methods of treating a frozen shoulder are provided. Such a method can include: targeting micro-tears within a portion of a capsule in the shoulder in ROI, directing therapeutic ultrasound energy to the micro-tears within a portion of a capsule in the shoulder, ablating at least a portion of the micro-tears within a portion of a capsule in the shoulder, and improving the micro-tears within a portion of a capsule in the shoulder. The method can include coupling ultrasound probe to ROI. The method can include focusing therapeutic ultrasound energy to create a lesion in a portion of the micro-tears within a portion of a capsule in the shoulder. The method can include creating a plurality of lesions in the micro-tears within a portion of a capsule in the shoulder. The method can include creating the plurality of lesion in a pattern, such as, a linear pattern, a 2-D pattern, or a 3-D pattern, and combinations thereof. The method further comprising measuring a distance on skin surface and then directing therapeutic ultrasound energy to the micro-tears within a portion of a capsule in the shoulder. The method can also include imaging micro-tears within a portion of a capsule in the shoulder. The method can also include imaging micro-tears within a portion of a capsule in the shoulder after the ablating at least a portion of the micro-tears within a portion of a capsule in the shoulder. The method can include comparing a measurement of the micro-tears within a portion of a capsule in the shoulder before and after the ablating step. The method can include directing acoustical pressure or cavitation to the micro-tears within a portion of a capsule in the shoulder after the ablating step further improving the micro-tears within a portion of a capsule in the shoulder. The method can include welding the micro-tears within a portion of a capsule in the shoulder with therapeutic ultrasound energy. The method can include increasing blood perfusion to the ROI. The method can include administering a medicant to the ROI.

Of course, the method described above for treating a frozen shoulder can be modified to treat any joint which is restricted in movement by an injured and/or inflamed capsule. For example, various embodiments provide a method for treating an injured capsule in a knee. In another example, various embodiments provide a method for treating an injured capsule in an ankle. In another example, various embodiments provide a method for treating an injured capsule in an elbow. Various embodiments provide a method for treating an injured capsule in any joint in a body.

In various embodiments, a method of treating a hyperextended capsule and/or partially torn capsule can include targeting the hyperextended capsule and/or partially torn capsule in ROI, directing therapeutic ultrasound energy 120 to the hyperextended capsule and/or partially torn capsule, ablating at least a portion of the inflamed tissue near or at a portion of a capsule in the shoulder, and improving the inflamed tissue near or at a portion of a capsule in the shoulder. The method can include coupling ultrasound probe to ROI. The method can include focusing therapeutic ultrasound energy to create a lesion in a portion of the hyperextended capsule and/or partially torn capsule. The method can include creating a plurality of lesions in the hyperextended capsule and/or partially torn capsule. The method can include creating the plurality of lesion in a pattern, such as, a linear pattern, a 2-D pattern, or a 3-D pattern, and combinations thereof. The method further comprising measuring a distance on skin surface 104 and then directing therapeutic ultrasound energy to the hyperextended capsule and/or partially torn capsule. The method can also include imaging a hyperextended capsule and/or partially torn capsule. The method can include directing acoustical pressure or cavitation to the hyperextended capsule and/or partially torn capsule after the ablating step further improving the hyperextended capsule and/or partially torn capsule hyperextended capsule and/or partially torn capsule. The method can include increasing blood perfusion to the ROI. The method can include administering a medicant to the ROI. According to various embodiments, methods of treating a hyperextended capsule and/or partially torn capsule. Such a method can include targeting micro-tears within a portion of a capsule in the shoulder in ROI, directing therapeutic ultrasound energy to the micro-tears within a portion of a capsule in the shoulder, ablating at least a portion of the micro-tears within a portion of a capsule in the shoulder, and improving the micro-tears within a portion of a capsule in the shoulder. The method can include coupling ultrasound probe to ROI. The method can include focusing therapeutic ultrasound energy to create a lesion in a portion of the micro-tears within a portion of a capsule in the shoulder. The method can include creating a plurality of lesions in the micro-tears within a portion of a capsule in the shoulder. The method can include creating the plurality of lesion in a pattern, such as, a linear pattern, a 2-D pattern, or a 3-D pattern, and combinations thereof. The method further comprising measuring a distance on skin surface 104 and then directing therapeutic ultrasound energy to the micro-tears within a portion of a capsule in the shoulder.

The method can also include imaging micro-tears within a portion of a capsule in the shoulder. The method can also include imaging micro-tears within a portion of a capsule in the shoulder after the ablating at least a portion of the micro-tears within a portion of a capsule in the shoulder. The method can include comparing a measurement of the micro-tears within a portion of a capsule in the shoulder before and after the ablating step. The method can include directing acoustical pressure or cavitation to the micro-tears within a portion of a capsule in the shoulder after the ablating step further improving the micro-tears within a portion of a capsule in the shoulder. The method can include welding the micro-tears within a portion of a capsule in the shoulder with therapeutic ultrasound energy. The method can include increasing blood perfusion to the ROI. The method can include administering a medicant to the ROI.

Of course, the method described above for treating a hyperextended capsule and/or partially torn capsule can be modified to treat any joint. For example, various embodiments provide a method for treating a hyperextended capsule and/or partially torn capsule in a knee. In another example, various embodiments provide a method for treating a hyperextended capsule and/or partially torn capsule in an ankle. In another example, various embodiments provide a method for treating a hyperextended capsule and/or partially torn capsule in an elbow. Various embodiments provide a method for treating a hyperextended capsule and/or partially torn capsule in any joint in a body.

Various embodiments provide a method for shrinking tissue in an inflamed capsule.

In some embodiments, cosmetic enhancement can refer to procedures, which are not medically necessary and are used to improve or change the appearance of a portion of the body. For example, a cosmetic enhancement can be a procedure but not limited to procedures that are used to improve or change the appearance of a nose, eyes, eyebrows and/or other facial features, or to improve or change the appearance and/or the texture and/or the elasticity of skin, or to improve or change the appearance of a mark or scar on a skin surface. According to various embodiments, method 100 results in cosmetic enhancement of a portion of the body.

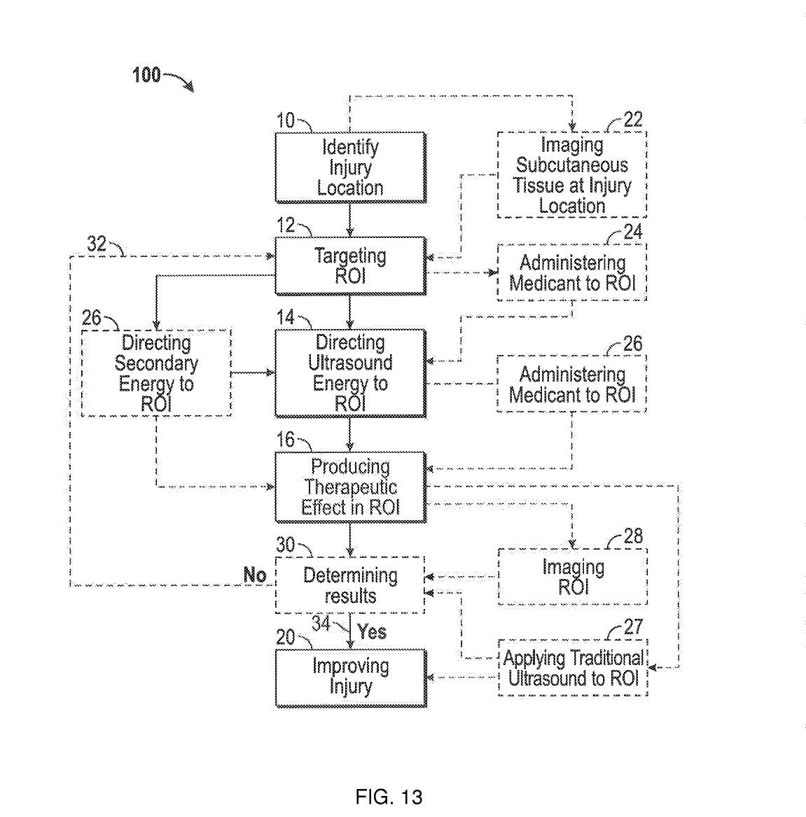

With reference to FIG. 13, a method of treatment is illustrated according to various embodiments. Step 10 is identifying the injury location. The injury location maybe anywhere in the body, such as, for example, in any of the following: leg, arm, wrist, hand, ankle, knee, foot, hip, shoulder, back, neck, chest, abdomen, and combinations thereof. Next, Step 12 is targeting a ROI. The ROI can be located in subcutaneous tissue below the skin surface of the injury location, which can be anywhere in the body, such as, those listed previously. In various embodiments, the ROI includes a portion of tissue in the joint. The muscle and connective layer can comprise any or all of the following tissues: muscle, tendon, ligament, and cartilage.

In various embodiments, the ROI comprises fibrous soft tissue. In some embodiments, the fibrous soft tissue is a muscle and connective tissue layer. In various embodiments, the fibrous soft tissue can comprise any or all of the following tissues: a muscle, a tendon, a ligament, fascia, a sheath, cartilage, and an articular capsule. In various embodiments, a muscle and connective layer is a fibrous connective layer, In various embodiments, the fibrous soft tissue is a fibrous connective tissue layer. In some embodiments, the fibrous soft tissue comprises a tendon. In some embodiments, the fibrous soft tissue comprises a tendon and a sheath. In some embodiments, the fibrous soft tissue comprises a tendon, a sheath, and a portion of muscle connected to the tendon. In some embodiments, the fibrous soft tissue comprises a tendon, fascia, and a muscle connected to the tendon. In some embodiments, the fibrous soft tissue comprises a ligament. In some embodiments, the fibrous soft tissue comprises a ligament and a portion of an articular capsule. In some embodiments, the fibrous soft tissue can include subcutaneous tissue surrounding fibrous connective tissue.

Optionally, step 22 is imaging subcutaneous tissue at the injury location and can be between steps 10 and 12 or can be substantially simultaneous with or be part of step 12.

After step 12, step 14 is directing therapeutic ultrasound energy to ROI. The therapeutic ultrasound energy may be focused or unfocused. The therapeutic ultrasound energy can be focused to a portion of tissue in the joint. The therapeutic ultrasound energy may ablate a portion of a portion of tissue in the joint. The therapeutic ultrasound energy may coagulate a portion of a portion of tissue in the joint. The therapeutic ultrasound energy can produce at least one lesion in a portion of tissue in the joint. The therapeutic ultrasound energy may micro-score a portion of a portion of tissue in the joint. The therapeutic ultrasound energy may be streaming. The therapeutic ultrasound energy may be directed to a first depth and then directed to a second depth. The therapeutic ultrasound energy may force a pressure gradient in a portion of tissue in the joint. The therapeutic ultrasound energy may be cavitation. The therapeutic ultrasound energy may be a first ultrasound energy effect, which comprises an ablative or a hemostatic effect, and a second ultrasound energy effect, which comprises at least one of non-thermal streaming, hydrodynamic, diathermic, and resonance induced tissue effects. Directing therapeutic ultrasound energy to the ROI is a non-invasive technique. As such, the layers above a portion of tissue in the joint are spared from injury. Such treatment does not require an incision in order to reach a portion of tissue in the joint to perform treatment for the injury.

In various embodiments, the ultrasound energy level for ablating a portion of tissue in a joint is in a range of about 0.1 joules to about 500 joules in order to create an ablative lesion. However, the ultrasound energy 108 level can be in a range of from about 0.1 joules to about 100 joules, or from about 1 joules to about 50 joules, or from about 0.1 joules to about 10 joules, or from about 50 joules to about 100 joules, or from about 100 joules to about 500 joules, or from about 50 joules to about 250 joules.

Further, the amount of time ultrasound energy is applied at these levels to create a lesion varies in the range from approximately 1 millisecond to several minutes. However, a range can be from about 1 millisecond to about 5 minutes, or from about 1 millisecond to about 1 minute, or from about 1 millisecond to about 30 seconds, or from about 1 millisecond to about 10 seconds, or from about 1 millisecond to about 1 second, or from about 1 millisecond to about 0.1 seconds, or about 0.1 seconds to about 10 seconds, or about 0.1 seconds to about 1 second, or from about 1 milliseconds to about 200 milliseconds, or from about 1 millisecond to about 0.5 seconds.

The frequency of the ultrasound energy can be in a range from about 0.1 MHz to about 100 MHz, or from about 0.1 MHz to about 50 MHz, or from about 1 MHz to about 50 MHz or about 0.1 MHz to about 30 MHz, or from about 10 MHz to about 30 MHz, or from about 0.1 MHz to about 20 MHz, or from about 1 MHz to about 20 MHz, or from about 20 MHz to about 30 MHz.

The frequency of the ultrasound energy can be in a range from about 1 MHz to about 12 MHz, or from about 5 MHz to about 15 MHz, or from about 2 MHz to about 12 MHz or from about 3 MHz to about 7 MHz.

In some embodiments, the ultrasound energy can be emitted to depths at or below a skin surface in a range from about 0 mm to about 150 mm, or from about 0 mm to about 100 mm, or from about 0 mm to about 50 mm, or from about 0 mm to about 30 mm, or from about 0 mm to about 20 mm, or from about 0 mm to about 10 mm, or from about 0 mm to about 5 mm. In some embodiments, the ultrasound energy can be emitted to depths below a skin surface in a range from about 5 mm to about 150 mm, or from about 5 mm to about 100 mm, or from about 5 mm to about 50 mm, or from about 5 mm to about 30 mm, or from about 5 mm to about 20 mm, or from about 5 mm to about 10 mm. In some embodiments, the ultrasound energy can be emitted to depths below a skin surface in a range from about 10 mm to about 150 mm, or from about 10 mm to about 100 mm, or from about 10 mm to about 50 mm, or from about 10 mm to about 30 mm, or from about 10 mm to about 20 mm, or from about 0 mm to about 10 mm.

In some embodiments, the ultrasound energy can be emitted to depths at or below a skin surface in the range from about 20 mm to about 150 mm, or from about 20 mm to about 100 mm, or from about 20 mm to about 50 mm, or from about 20 mm to about 30 mm. In some embodiments, the ultrasound energy can be emitted to depths at or below a skin surface in a range from about 30 mm to about 150 mm, or from about 30 mm to about 100 mm, or from about 30 mm to about 50 mm. In some embodiments, the ultrasound energy can be emitted to depths at or below a skin surface in a range from about 50 mm to about 150 mm, or from about 50 mm to about 100 mm. In some embodiments, the ultrasound energy can be emitted to depths at or below a skin surface in a range from about 20 mm to about 60 mm, or from about 40 mm to about 80 mm, or from about 10 mm to about 40 mm, or from about 5 mm to about 40 mm, or from about 0 mm to about 40 mm, or from about 10 mm to about 30 mm, or from about 5 mm to about 30 mm, or from about 0 mm to about 30 mm.

In various embodiments, a temperature of tissue receiving the ultrasound energy can be in a range from 30° C. to about 100° C., or from 43° C. to about 60° C., or from 50° C. to about 70° C., or from 30° C. to about 50° C., or from 43° C. to about 100° C., or from 33° C. to about 100° C., or from 30° C. to about 65° C., or from 33° C. to about 70° C., as well as variations thereof.

Optionally, step 24, which is administering a medicant to the ROI, can be between steps 12 and 14. The medicant can be any chemical or naturally occurring substance that can assist in treating the injury. For example the medicant can be an anti-inflammant, or a steroid, or a blood vessel dilator. The medicant can be administered by applying it to the skin above the ROI. The medicant can be administered to the circulatory system. For example, the medicant can be in the blood stream and can be activated or moved to the ROI by the therapeutic ultrasound energy. Any naturally occurring proteins, stem cells, growth factors and the like can be used as medicant in accordance to various embodiments. A medicant can be mixed in a coupling gel or can be used as a coupling gel. Medicants are further discussed herein.

Step 16 is producing a therapeutic effect in the ROI. A therapeutic effect can be cauterizing and repairing a portion of tissue in the joint. A therapeutic effect can be stimulating or increase an amount of beat shock proteins. Increasing temperature of the joint can stimulate a change to at least one of a concentration and an activity of growth factors and/or heat shock proteins in the joint. Such a therapeutic effect can cause white blood cells to promote healing of a portion of the muscle and connective layer in the ROI. A therapeutic effect can be peaking inflammation in a portion of the ROI to decrease pain at the injury location. Peaking inflammation can cause suppression of the immune system around and in the joint. Peaking inflammation can accelerate a healing cascade, such as, for example, the coagulation cascade.

A therapeutic effect can be creating lesion to restart or increase the wound healing cascade at the injury location. A therapeutic effect can be increasing the blood perfusion to the injury location which can accelerate healing at the site. Such a therapeutic effect would not require ablative ultrasound energy. A therapeutic effect can be encouraging collagen growth. A therapeutic effect can be relieving pain. A therapeutic effect may increase the “wound healing” response through the liberation of cytokines and may produce reactive changes within the tendon and muscle itself, helping to limit surrounding tissue edema and decrease an inflammatory response to an injury to a joint. A therapeutic effect can be synergetic with the medicant administered to ROI in steps 24 and/or 26. A therapeutic effect can be healing an injury to a muscle. A therapeutic effect can be repairing a tendon. A therapeutic effect can be repairing a ligament A therapeutic effect can be repairing a muscle and a tendon connected to the muscle. Therapeutic effects can be combined.

Optionally, step 26, which is administering medicant to ROI, can be between steps 14 and 16 or can be substantially simultaneous with or be part of step 16. The medicants useful in step 26 are essentially the same as those discussed for step 24.

Optionally, after step 12, step 25, which is directing secondary energy to the ROI can be substantially simultaneous with or be part of step 16. However, step 25 can be administered at least one of before and after step 16. Step 25 can be alternated with step 16, which can create a pulse of two different energy emissions to the ROI. Secondary energy can be provided by a laser source, or an intense pulsed light source, or a light emitting diode, or a radio frequency, or a plasma source, or a magnetic resonance source, or a mechanical energy source, or any other photon-based energy source. Secondary energy can be provided by any appropriate energy source now known or created in the future. More than one secondary energy source may be used for step 25.

Furthermore, various embodiments provide energy, which may be a first energy and a second energy. For example, a first energy may be followed by a second energy, either immediately or after a delay period. In another example, a first energy and a second energy can be delivered simultaneously. In some embodiments, the first energy and the second energy is ultrasound energy. In some embodiments, the first energy is ultrasound and the second energy is generated by one of a laser, an intense pulsed light, a light emitting diode, a radiofrequency generator, photon-based energy source, plasma source, a magnetic resonance source, or a mechanical energy source, such as for example, pressure, either positive or negative. In other embodiments, energy may be a first energy, a second energy, and a third energy, emitted simultaneously or with a time delay or a combination thereof. In some embodiments, energy may be a first energy, a second energy, a third energy, and an nth energy, emitted simultaneously or with a time delay or a combination thereof. Any of the a first energy, a second energy, a third energy, and a nth maybe generated by at least one of a laser, an intense pulsed light, a light emitting diode, a radiofrequency generator, an acoustic source, photon-based energy source, plasma source, a magnetic resonance source, and/or a mechanical energy source.

Step 20 is improving the injury. Optionally, between steps 16 and 20 is step 30, which is determining results. Between steps 16 and 30 is option step 28, which is imaging the ROI. The images of the ROI from step 28 can be useful for the determining results of step 30. If the results of step 30 are acceptable within the parameters of the treatment then Yes direction 34 is followed to step 20. If the results of step 30 are not acceptable within the parameters of the treatment then No direction 32 is followed back to step 12. After step 16, optionally traditional ultrasound heating can be applied to the ROI in step 27. This application of traditional ultrasound heating to the ROI can be useful in keeping a medicant active or providing heat to support blood perfusion to the ROI after step 16, Further examples and variations of treatment method 100 are discussed herein.

In addition, various different subcutaneous tissues, including for example, muscle and connective layer, may be treated by method 100 to produce different bin-effects, according to some embodiments of the present disclosure. Furthermore, any portion of a joint may be treated by method 100 to produce one or more bio-effects, as described herein, in accordance to various embodiments. In order to treat a specific injury location and to achieve a desired bio-effect, therapeutic ultrasound energy may be directed to a specific depth within ROI to reach the targeted subcutaneous tissue, such as, for example, muscle and connective layer. For example, if it is desired to cut muscle by applying therapeutic ultrasound energy 120 at ablative levels, which may be approximately 5 mm to 15 mm below skin surface or at other depths as described herein. An example of ablating muscle can include a series of lesions ablated into muscle. Besides ablating a portion of tissue in the joint, other bio-effects may comprise incapacitating, partially incapacitating, severing, rejuvenating, removing, ablating, micro-ablating, shortening, manipulating, or removing tissue either instantly or over time, and combinations thereof.

Depending at least in part upon the desired bio-effect and the subcutaneous tissue being treated, method 100 may be used with an extracorporeal, non-invasive procedure. Also, depending at least in part upon the specific bio-effect and tissue targeted, temperature may increase within ROI may range from approximately 30° C.′ to about 100° C., or in a range from about 30° C. to about 100° C., or in other appropriate temperature ranges that are described herein.

Other bio-effects to target tissue, such as, a portion of tissue in the joint, can include heating, cavitation, steaming, or vibro-accoustic stimulation, and combinations thereof. In various embodiments, therapeutic ultrasound energy is deposited in a matrices of micro-coagulative zones to an already injured tendon or muscle can increase the “wound healing” response through the liberation of cytokines and may produce reactive changes within the tendon and muscle itself, helping to limit surrounding tissue edema and decrease the inflammatory response to an injury to a joint. In various embodiments, therapeutic ultrasound energy is deposited in a matrices of micro-coagulative zones to an already injured tendon or muscle changes at least one of concentration and activity of inflammatory mediators (such as but not limited to TNF-A, IL-1) as well as growth factors (such as but not limited to TGF-B1, TGF-B3) at the site of the injure tendon or muscle.

In various embodiments, therapeutic ultrasound energy is deposited in a matrices of micro-coagulative zones to an already injured tendon or muscle, which can stimulate a change in at least one of concentration and activity of one or more of the following: Adrenomedullin (AM), Autocrine motility factor, Bone morphogenetic proteins (BMPs), Brain-derived neurotrophic factor (BDNF), Epidemal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony stimulating factor (GM-CSF), Growth differentiation factor-9 (GDF9), Hepatocyte growth factor (HGF), Hepatoma-derived growth factor (HDGF), Insulin-like growth factor (IGF), Migrationstimulating factor, Myostatin (GDF-8), Nerve growth factor (NGF) and other neurotrophins, Platelet-derived growth factor (PDGF), Thrombopoietin (TPO), Transforming growrth factor alpha(TGF-α), Transforming growth factor beta(TGF-β), Tumor necrosis factor-alpha(TNFα), Vascular endothelial growth factor (VEGF), Wnt Signaling Pathway, placental growth factor (PIGF), [(Foetal Bovine Somatotrophin)] (FBS), IL-1-Cofactor for IL-3 and IL-6, which can activate T cells, IL-2-T-cell growth factor, which can stimulate IL-1 synthesis and can activate B-cells and NK cells, IL-3, which can stimulate production of all nonlymphoid cells, IL-4-Growth factor for activating B cells, resting T cells, and mast cells, IL-5, which can induce differentiation of activated B cells and eosinophils, IL-6, which can stimulate Ig synthesis and growth factor for plasma cells, IL-7 growth factor for pre-B cells, and/or any other growth factor not listed herein, and combinations thereof.

Further, medicants, as described above, can include a drug, a medicine, or a protein, and combinations thereof. Medicants can also include adsorbent chemicals, such as zeolites, and other hemostatic agents are used in sealing severe injuries quickly. Thrombin and fibrin glue are used surgically to treat bleeding and to thrombose aneurysms. Medicants can include Desmopressin is used to improve platelet function by activating arginine vasopressin receptor 1A. Medicants can include coagulation factor concentrates are used to treat hemophilia, to reverse the effects of anticoagulants, and to treat bleeding in patients with impaired coagulation factor synthesis or increased consumption. Prothrombin complex concentrate, cryoprecipitate and fresh frozen plasma are commonly-used coagulation factor products. Recombinant activated human factor VII can be used in the treatment of major bleeding. Medicants can include tranexamic acid and aminocaproic acid, can inhibit fibrinolysis, and lead to a de facto reduced bleeding rate. In addition, medicants can include steroids like the glucocorticoid cortisol.

According to various embodiments of method 100, ultrasound probe is coupled directly to ROI, as opposed to skin surface 104, to treat targeted tissue. For example, ultrasound probe can be integrated to or attached to a tool, such as, for example, an arthroscopic tool, laparoscopic tool, or an endoscopic tool that may be inserted into a patient's body with minimal invasiveness.

In various embodiments, method 100 can treat either recent or older injuries, or combinations thereof Inflammation can be classified as either acute or chronic. Acute inflammation is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process. in various embodiments, method 100 can treat chronic inflammation. In various embodiments, method 100 can treat acute inflammation. In some embodiments, method 100 can treat a combination of acute and chronic inflammation.

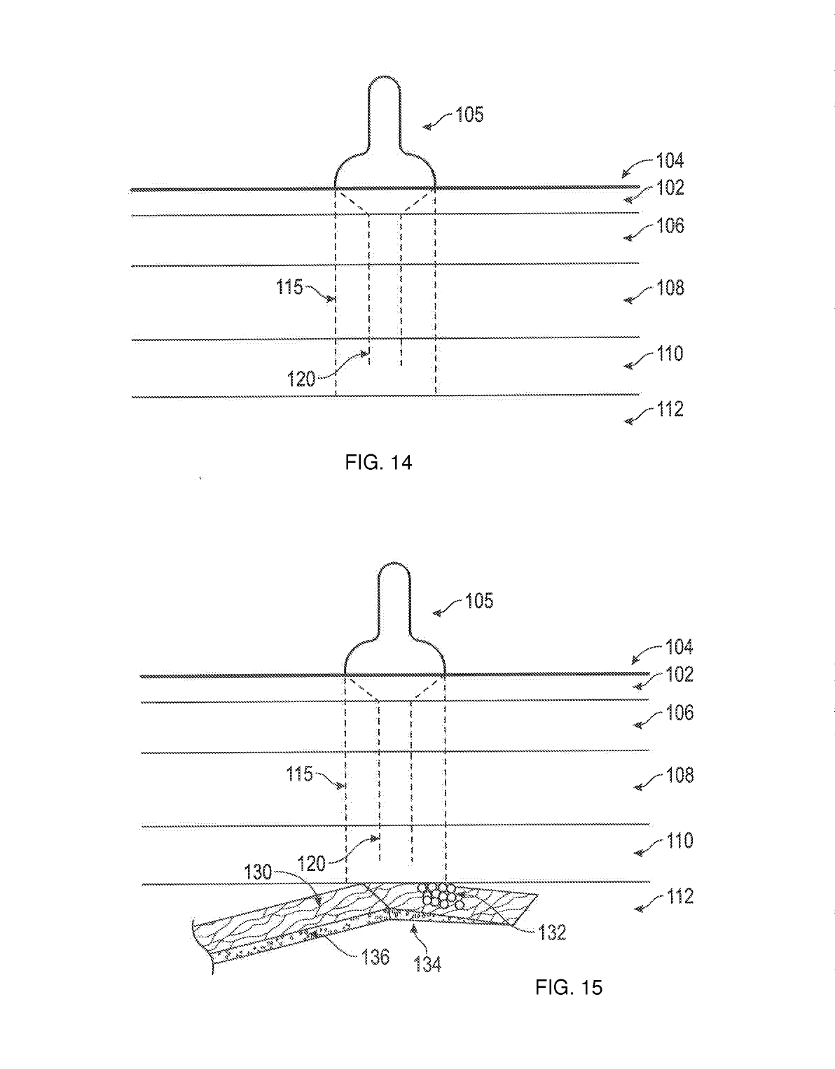

Now moving to FIG. 14, a cross sectional view of tissue layers and ultrasound energy directed to a portion of tissue in the joint, according to various embodiments, is illustrated. Typically, ultrasound energy propagates as a wave with relatively little scattering, over depths up to many centimeters in tissue depending on the ultrasound frequency. The focal spot size achievable with any propagating wave energy, depends on wavelength. Ultrasound wavelength is equal to the acoustic velocity divided by the ultrasound frequency. Attenuation (absorption, mainly) of ultrasound by tissue also depends on frequency Shaped lesion can be created through adjustment of the strength, depth, and type of focusing, energy levels and timing cadence. For example, focused ultrasound can be used to create precise arrays of microscopic thermal ablation zones. Ultrasound energy 120 can produce an array of ablation zones deep into the layers of the soft tissue. Detection of changes in the reflection of ultrasound energy can be used for feedback control to detect a desired effect on the tissue and used to control the exposure intensity, time, and/or position. In various embodiment, ultrasound probe 105 is configured with the ability to controllably produce conformal lesions of thermal injury in soft tissue within ROI 115 through precise spatial and temporal control of acoustic energy deposition, i.e., control of ultrasound probe 105 is confined within selected time and space parameters, with such control being independent of the tissue. The ultrasound energy 120 can be controlled using spatial parameters. The ultrasound energy 120 can be controlled using temporal parameters. The ultrasound energy 120 can be controlled using a combination of temporal parameters and spatial parameters.

In accordance with various embodiments, control system and ultrasound probe 105 can be configured for spatial control of ultrasound energy 120 by controlling the manner of distribution of the ultrasound energy 120. For example, spatial control may be realized through selection of the type of one or more transducer configurations insonifying ROI 115, selection of the placement and location of ultrasound probe 105 for delivery of ultrasound energy 120 relative to ROI 115 e.g., ultrasound probe 105 being configured for scanning over part or whole of ROI 115 to produce contiguous thermal injury having a particular orientation or otherwise change in distance from ROI 115, and/or control of other environment parameters, e.g., the temperature at the acoustic coupling interface can be controlled, and/or the coupling of ultrasound probe 105 to tissue. Other spatial control can include but are not limited to geometry configuration of ultrasound probe 105 or transducer assembly, lens, variable focusing devices, variable focusing lens, stand-offs, movement of ultrasound probe, in any of six degrees of motion, transducer backing, matching layers, number of transduction elements in transducer, number of electrodes, or combinations thereof. In various embodiments, control system and ultrasound probe 105 can also be configured for temporal control, such as through adjustment and optimization of drive amplitude levels, frequency, waveform selections, e.g., the types of pulses, bursts or continuous waveforms, and timing sequences and other energy drive characteristics to control thermal ablation of tissue. Other temporal control can include but are not limited to full power burst of energy, shape of burst, timing of energy bursts, such as, pulse rate duration, continuous, delays, etc., change of frequency of burst, burst amplitude, phase, apodization, energy level or combinations thereof.

The spatial and/or temporal control can also be facilitated through open-loop and closed-loop feedback arrangements, such as through the monitoring of various spatial and temporal characteristics. As a result, control of acoustical energy within six degrees of freedom, e.g., spatially within the X, Y and Z domain, as well as the axis of rotation within the XY, YZ and XZ domains, can be suitably achieved to generate conformal lesions of variable shape, size and orientation. For example, through such spatial and/or temporal control, ultrasound probe 105 can enable the regions of thermal injury to possess arbitrary shape and size and allow the tissue to be destroyed (ablated) in a controlled manner.

The tissue layers illustrated in FIG. 14 are skin surface 104, epidermal layer 102, dermis layer 106, fat layer 108, SMAS layer 110, and muscle and connective tissue layer 112. Ultrasound probe 105 emits therapeutic ultrasound energy 120 in ROI 115. In various embodiments, ultrasound probe 105 is capable of emitting therapeutic ultrasound energy 120 at variable depths in ROI 115, such as, for example, the depths described herein. Ultrasound probe 105 is capable of emitting therapeutic ultrasound energy as a single frequency, variable frequencies, or a plurality of frequencies, such as, for example, the frequency ranges described herein. Ultrasound probe 105 is capable of emitting therapeutic ultrasound energy 120 for variable time periods or to pulse the emission over time, such as, for example, those time intervals described herein. Ultrasound probe 105 is capable of providing various energy levels of therapeutic ultrasound energy, such as, for example, the energy levels described herein. Ultrasound probe 105 may be individual hand-held device, or may be part of a treatment system. The ultrasound probe 105 can provide both therapeutic ultrasound energy and imaging ultrasound energy. However, ultrasound probe 105 may provide only therapeutic ultrasound energy. Ultrasound probe 105 may comprise a therapeutic transducer and a separate imaging transducer. Ultrasound probe 105 may comprise a transducer or a transducer array capable of both therapeutic and imaging applications. Accordingly an alternative embodiment, ultrasound probe 105 is coupled directly to one of the tissue layers, as opposed to skin surface 104 to treat the tissue layer. For example, ultrasound probe can be integrated to or attached to a tool, such as, for example, an arthroscopic tool, laparoscopic tool, or an endoscopic tool that may be inserted into a patient's body with minimal invasiveness.

In various embodiments, ultrasound probe 105 may be used for method 100. In various embodiments, method 100 can be implemented using any or all of the elements illustrated in FIG. 14. As will be appreciated by those skilled in the art, at least a portion of method 100 or a variation of method 100 can be implemented using any or all of the elements illustrated in FIG. 14.

With reference to FIG. 15, a cross sectional view of tissue layers and ultrasound energy directed to at least one of muscle 130 and tendon 134, according to various embodiments, is illustrated. The tissue layers illustrated are skin surface 104, epidermal layer 102, dermis layer 106, fat layer 108, SMAS layer 110, tendon 134, muscle 130, and fat 132. In some embodiments, ROI 115 comprises at least one of muscle 130 and tendon 134. In some embodiments, ROI 115 can comprise skin surface 104, epidermal layer 102, dermis layer 106, fat layer 108, SMAS layer 110, and muscle and connective tissue layer 112, which comprises tendon 134, muscle 130, and fat 132. In some embodiments, ultrasound probe 105 images at least a portion of one of skin surface 104, epidermal layer 102, dermis layer 106, fat layer 108, SMAS layer 110, and muscle and connective tissue layer 112, which comprises tendon 134, muscle 130, and fat 132. In some embodiments, ultrasound probe 105 images at least one muscle 130 and tendon 134. Ultrasound probe 105 emits therapeutic ultrasound energy 120 to at least one of muscle 130 and tendon 134. As well known to those skilled in the art, tendon 134 attaches muscle 130 to bone 136. In various embodiments, therapeutic ultrasound energy 120 treats at least one of muscle 130 and tendon 134. In some embodiments, therapeutic ultrasound energy 120 ablates a portion of at least one a muscle 130 and tendon 134 creating a lesion. In some embodiments therapeutic ultrasound energy 120 coagulates a portion of at least one of muscle 130 and tendon 134. Accordingly an alternative embodiment, ultrasound probe 105 is coupled directly to a portion of at least one of muscle 130 and tendon 134, as opposed to skin surface 104, to treat the a portion of at least one of muscle 130 and tendon 134. For example, ultrasound probe can be integrated to or attached to a tool, such as, for example, an arthroscopic tool, laparoscopic tool, or an endoscopic tool that may be inserted into a patient's body with minimal invasiveness,

The tissue layers illustrated are skin surface 104, epidermal layer 102, dermis layer 106, fat layer 108, S MAS layer 110, and muscle and connective tissue layer 112, which comprises cartilage 140 and ligament 138. As well known to those skilled in the art, joint 135 can comprise ligament 138, cartilage 140, and bone 136. In some embodiments, ROI 115 comprises at least one of cartilage 140 and ligament 138. In some embodiments, ROI 115 can comprise at least a portion of joint 135. ROI 115 can comprise any or all of the following: skin surface 104, epidermal layer 102, dermis layer 106, fat layer 108, SMAS layer 110, and muscle and connective tissue 112, which comprises ligament 138 and cartilage 140. In some embodiments, ultrasound probe 105 can image at least a portion of one of skin surface 104, epidermal layer 102, dermis layer 106, fat layer 108, SMAS layer 110, ligament 138 and cartilage 140. Ultrasound probe 105 emits therapeutic ultrasound energy 120 to at least one of ligament 138 and cartilage 140. In various embodiments, therapeutic ultrasound energy 120 treats at least one of ligament 138 and cartilage 140. In various embodiments, therapeutic ultrasound energy 120 treats at least a portion of joint 135.

In some embodiments, therapeutic ultrasound energy 120 ablates a portion of cartilage 140 creating a lesion. In some embodiments, therapeutic ultrasound energy 120 ablates a portion of joint 135 creating a lesion. In some embodiments therapeutic ultrasound energy coagulates a portion of cartilage 140. In some embodiments therapeutic ultrasound energy 120 coagulates a portion of joint 135. In some embodiments, therapeutic ultrasound energy 120 regenerates cartilage 140. In some embodiments, therapeutic ultrasound energy 120 ablates a portion of cartilage 140. In some embodiments, therapeutic ultrasound energy 120 increases perfusion of blood to a portion of cartilage 140. In some embodiments, therapeutic ultrasound energy 120 welds torn cartilage 140 to repair a tear in cartilage 140.

In some embodiments, ultrasound probe 105 can be removed in at least one direction to provide a plurality of lesions 25 in cartilage 140. In various embodiments, a plurality of lesions 25 can be placed in a pattern in a portion of cartilage 140, such as, for example, a 1-D pattern, a 2-D pattern, a 3-D pattern, or combinations thereof. In some embodiments, therapeutic ultrasound energy 120 ablates a portion muscle 130 creating lesion 25. In some embodiments, therapeutic ultrasound energy 120 ablates a portion muscle 130 creating lesion 25. In some embodiments, therapeutic ultrasound energy 120 coagulates a portion of muscle 130.

Therapeutic ultrasound energy 120 creates ablation zone in a tissue layer, at which a temperature of tissue is raised to at least 43° C., or is raised to a temperature in the range form about 43° C. to about 100° C., or from about 50° C. to about 90° C., or from about 55° C. to about 75° C., or from about 50° C. to about 65° C., or from about 60° C. to about 68° C. In some embodiments, ultrasound probe 105 can be moved in at least one direction to provide a plurality of lesions 25 in a tissue layer. In various embodiments, a plurality of lesions 25 can be placed in a pattern in at least one tissue layer, such as, for example, a 1-D pattern, a 2-D pattern, a 3-D pattern, or combinations thereof. In some embodiments, ultrasound probe 105 comprises a single transducer element and while emitting therapeutic ultrasound energy 120 in a pulsed matter, is moved in a linear motion along skin surface 104 to create a 1-D pattern of a plurality of lesions 25 in at least one tissue layer. In some embodiments, ultrasound probe 105 comprises a linear array of transducers and while emitting therapeutic ultrasound energy 120 in a pulsed matter, is moved along the linear vector of the array on skin surface 104 to create a 1-D pattern of a plurality of lesions 25 in at least one tissue layer.

In various embodiments, ultrasound probe 105 may be used for method 100. In various embodiments, method 100 can be implemented using any or all of the elements illustrated in FIG. 15. As will be appreciated by those skilled in the art, at least a portion of method 100 or a variation of method 100 can be implemented using any or all of the elements illustrated in FIG. 15.

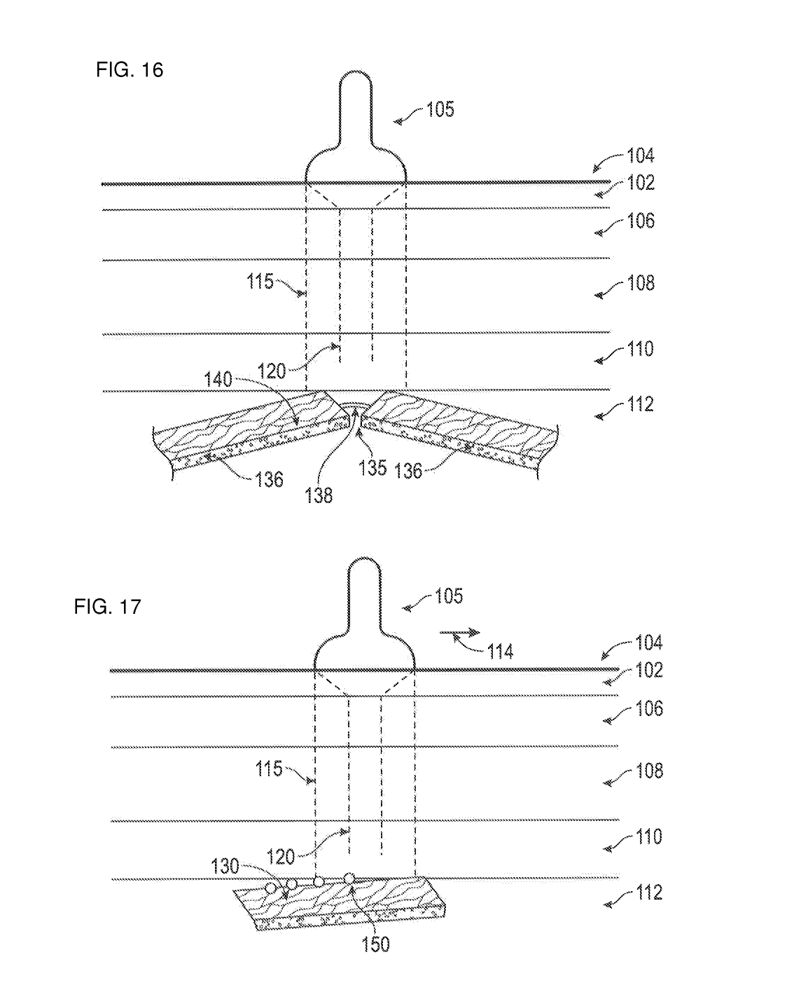

In FIG. 16, a cross sectional view of tissue layers and ultrasound energy directed to at least one of cartilage 140 and ligament 138, according to various embodiments, is illustrated. The tissue layers illustrated are skin surface 104, epidermal layer 102, dermis layer 106, fat layer 108, SMAS layer 110, and muscle and connective tissue layer 112, which comprises cartilage 140 and ligament 138. As well known to those skilled in the art, joint 135 can comprise ligament 138, cartilage 140, and bone 136. In some embodiments, ROI 115 comprises at least one of cartilage 140 and ligament 138. In some embodiments, ROI 115 can comprise at least a portion of joint 135. ROI 115 can comprise any or all of the following: skin surface 104, epidermal layer 102, dermis layer 106, fat layer 108, SMAS layer 110, and muscle and connective tissue 112, which comprises ligament 138 and cartilage 140. In some embodiments, ultrasound probe 105 can image at least a portion of one of skin surface 104, epidermal layer 102, dermis layer 106, fat layer 108, SMAS layer 110, ligament 138 and cartilage 140. Ultrasound probe 105 emits therapeutic ultrasound energy 120 to ligament 138. In various embodiments, therapeutic ultrasound energy 120 treats ligament 138. In various embodiments, therapeutic ultrasound energy 120 treats at least a portion of joint 135. According an alternative embodiment, ultrasound probe 105 is coupled directly to a portion of joint 135, as opposed to skin surface 104, to treat the a portion of joint 135. For example, ultrasound probe can be integrated to or attached to a tool, such as, for example, an arthroscopic tool, laparoscopic tool, or an endoscopic tool that may be inserted into a patient's body with minimal invasiveness. In various embodiments, ultrasound probe 105 may be used for method 100. In various embodiments, method 100 can be implemented using any or all of the elements illustrated in FIG. 16. As will be appreciated by those skilled in the art, at least a portion of method 100 or a variation of method 100 can be implemented using any or all of the elements illustrated in FIG. 16.

In some embodiments, therapeutic ultrasound energy 120 ablates a portion of a ligament 138 creating a lesion. In some embodiments, therapeutic ultrasound energy ablates a portion of joint 135 creating a lesion. In some embodiments therapeutic ultrasound energy coagulates a portion of ligament 138. In some embodiments therapeutic ultrasound energy 120 coagulates a portion of joint 135.

Referring to FIG. 17, a cross sectional view of tissue layers and ultrasound energy creating a plurality of lesions in muscle tissue, according to various embodiments of the present invention, is illustrated. The tissue layers illustrated are skin surface 104, epidermal layer 102, dermis layer 106, fat layer 108, SMAS layer 110, and muscle 130. In some embodiments, ROI 115 comprises a portion of muscle 130. In some embodiments, ROI 115 can comprise skin surface 104, epidermal layer 102, dermis layer 106, fat layer 108, SMAS layer 110, and muscle and connective tissue layer 112, which comprises at least a portion of muscle 130. In some embodiments, ultrasound probe 105 images at least a portion of one of skin surface 104, epidermal layer 102, dermis layer 106, fat layer 108, SMAS layer 110, and muscle and connective tissue layer 112, which comprises at least a portion of muscle 130. In some embodiments, ultrasound probe 105 images at least a portion of muscle 130. Ultrasound probe 105 emits therapeutic ultrasound energy 120 to at least a portion of muscle 130. In various embodiments, therapeutic ultrasound energy 120 treats a portion of muscle 130. In various embodiments, ultrasound probe 105 may be used for method 100. In various embodiments, method 100 can be implemented using any or all of the elements illustrated in FIG. 17. As will be appreciated by those skilled in the art, at least a portion of method 100 or a variation of method 100 can be implemented using any or all of the elements illustrated in FIG. 17.

In some embodiments, ultrasound probe 105 can be moved in at least one direction 114 to provide a plurality of lesions 25 in muscle 130. In various embodiments, a plurality of lesions 25 can be placed in a pattern in a portion of muscle 130, such as, for example, a 1-D pattern, a 2-D pattern, a 3-D pattern, or combinations thereof. In some embodiments, therapeutic ultrasound energy 120 ablates a portion muscle 130 creating lesion 25. In some embodiments, therapeutic ultrasound energy 120 ablates a portion muscle 130 creating lesion 25. In some embodiments, therapeutic ultrasound energy 120 coagulates a portion of muscle 130.

Therapeutic ultrasound energy 120 creates ablation zone 150 in a tissue layer, at which a temperature of tissue is raised to at least 43° C., or is raised to a temperature in the range form about 43° C. to about 100° C., or from about 50° C. to about 90° C., or from about 55° C. to about 75° C., or from about 50° C. to about 65° C., or from about 60° C. to about 68° C.

In some embodiments, ultrasound probe 105 can be moved in at least one direction 114 to provide a plurality of lesions 25 in a tissue layer. In various embodiments, a plurality of lesions 25 can be placed in a pattern in at least one tissue layer, such as, for example, a 1-D pattern, a 2-D pattern, a 3-D pattern, or combinations thereof. In some embodiments, ultrasound probe 105 comprises a single transducer element and while emitting therapeutic ultrasound energy 120 in a pulsed matter, is moved in a linear motion along skin surface 104 to create a 1-D pattern of a plurality of lesions 25 in at least one tissue layer. In some embodiments, ultrasound probe 105 comprises a linear array of transducers and while emitting therapeutic ultrasound energy 120 in a pulsed matter, is moved along the linear vector of the array on skin surface 104 to create a 1-D pattern of a plurality of lesions 25 in at least one tissue layer.

In some embodiments, ultrasound probe 105 comprises a linear array of transducers and while emitting therapeutic ultrasound energy 120 in a pulsed matter, is moved along the non-linear vector of the array on skin surface 104 to create a 2-D pattern of a plurality of lesions 25 in at least one tissue layer. In some embodiments, ultrasound probe 105 comprises an array of transducers and while emitting therapeutic ultrasound energy 120 in a pulsed matter, is moved along skin surface 104 to create a 2-D pattern of a plurality of lesions 25 in at least one tissue layer.

In some embodiments, ultrasound probe 105 comprises an array of transducers, wherein the array comprises a first portion focusing to a first depth and a second portion focusing to a second depth, and while emitting therapeutic ultrasound energy 120 in a pulsed matter, is moved along skin surface 104 to create a 3-D pattern of a plurality of lesions 25 in at least one tissue layer. In some embodiments, ultrasound probe 105 comprises at least two arrays of transducers, wherein a first array focusing to a first depth and a second array focusing to a second depth, and while each of the arrays emitting therapeutic ultrasound energy 120 in a pulsed matter, is moved along skin surface 104 to create a 3-D pattern of a plurality of lesions 25 in at least one tissue layer. In some embodiments, ultrasound probe 105 comprises a linear array of transducers and while emitting therapeutic ultrasound energy 120 in a pulsed matter, is moved along the non-linear vector of the array on skin surface 104 focused to a first depth then moved in the same direction along skin surface focused at a second depth to create a 3-D pattern of a plurality of lesions 25 in at least one tissue layer. In some embodiments, ultrasound probe 105 comprises an array of transducers and while emitting therapeutic ultrasound energy 120 in a pulsed matter, is moved along skin surface 104 focused to a first depth then moved in the same direction along skin surface focused at a second depth to create a 3-D pattern of a plurality of lesions 25 in at least one tissue layer.

In various embodiments, methods of building muscle are provided. The method can include targeting the muscle 130 to be strengthened, directing therapeutic ultrasound energy to the muscle 130, creating a pattern of a plurality of lesions 25, allowing the muscle to heal, thereby strengthening the muscle 130. In addition, such methods can useful for building muscle 130 mass. Still further, such methods can be useful for treating stroke victims.

A tendon is a tough yet flexible band of fibrous connective tissue that usually connects muscle to bone, it transmits the force of the muscle contraction to the bone which enables movement. Normal healthy tendons are composed of parallel arrays of collagen fibers closely packed together. The fibers are mostly collagen type I, however, both collagen type III and V may be present. Collagen molecules are produced by tenocytes and aggregate end-to-end and side-to side to produce collagen fibrils, organized fibril bundles form fibers, groups of fibers form macroaggregates, groups of macroaggregates bounded by endotendon form fascicles and groups of fascicles bounded by epitendon and peritendon form the tendon organ.

The specific configurations of controlled thermal injury are selected to achieve the desired tissue and therapeutic effect. For example, any tissue effect can be realized, including but not limited to thermal and non-thermal streaming, cavitational, hydrodynamic, ablative, hemostatic, diathermic, and/or resonance-induced tissue effects. Additional embodiments useful for creating lesions may be found in US Patent Publication No. 20060116671 entitled “Method and System for Controlled Thermal Injury of Human Superficial Tissue” published Jun. 1, 2006 and incorporated by reference.

In various embodiments, methods, described herein, can stimulate coagulation by depositing target ultrasound energy with or without a medicant. Coagulation is a complex process by which blood forms clots. It is an important part of hemostasis (the cessation of blood loss from a damaged vessel), wherein a damaged blood vessel wall is covered by a platelet and fibrin-containing clot to stop bleeding and begin repair of the damaged vessel Disorders of coagulation can lead to an increased risk of bleeding (hemorrhage) or obstructive clotting (thrombosis).

Coagulation begins almost instantly after an injury to the blood vessel has damaged the endothelium (lining of the vessel). Exposure of the blood to proteins such as tissue factor initiates changes to blood platelets and the plasma protein fibrinogen, a clotting factor. Platelets immediately form a plug at the site of injury; this is called primary hemostasis. Secondary hemostasis occurs simultaneously: Proteins in the blood plasma, called coagulation factors or clotting factors, respond in a complex cascade to form fibrin strands, which strengthen the platelet plug.

In some embodiments, methods, described herein, can initiate coagulation cascade by depositing target ultrasound energy with or without a medicant. The coagulation cascade of secondary hemostasis has two pathways which lead to fibrin formation. These are the contact activation pathway (formerly known as the intrinsic pathway), and the tissue factor pathway (formerly known as the extrinsic pathway). It was previously thought that the coagulation cascade consisted of two pathways of equal importance joined to a common pathway. It is now known that the primary pathway for the initiation of blood coagulation is the tissue factor pathway. The pathways are a series of reactions, in which a zymogen (inactive enzyme precursor) of a serine protease and its glycoprotein co-factor are activated to become active components that then catalyze the next reaction in the cascade, ultimately resulting in cross-linked fibrin.

The coagulation factors are generally serine proteases (enzymes). There are some exceptions. For example, FVIII and FV are glycoproteins, and Factor XIII is a transglutaminase. Serine proteases act by cleaving other proteins at specific sites. The coagulation factors circulate as inactive zymogens. The coagulation cascade is classically divided into three pathways. The tissue factor and contact activation pathways both activate the “final common pathway” of factor X, thrombin and fibrin.

Soon after injury, a wound healing cascade is unleashed. This cascade is usually said to take place in three phases: the inflammatory, proliferative, and maturation stages.

In some embodiments, methods, described herein, can peak inflammation by depositing target ultrasound energy with or without a medicant in the inflammatory phase, macrophages and other phagocytic cells kill bacteria, debride damaged tissue and release chemical factors such as growth hormones that encourage fibroblasts, epithelial cells and endothelial cells which make new capillaries to migrate to the area and divide.

In the proliferative phase, immature granulation tissue containing plump active fibroblasts forms. Fibroblasts quickly produce abundant type HI collagen, which fills the defect left by an open wound. Granulation tissue moves, as a wave, from the border of the injury towards the center.

As granulation tissue matures, the fibroblasts produce less collagen and become more spindly in appearance. They begin to produce the much stronger type I collagen. Some of the fibroblasts mature into myofibroblasts which contain the same type of actin found in smooth muscle, which enables them to contract and reduce the size of the wound.

During the maturation phase of wound healing, unnecessary vessels fanned in granulation tissue are removed by apoptosis, and type III collagen is largely replaced by type I. Collagen which was originally disorganized is cross-linked and aligned along tension lines. This phase can last a year or longer. Ultimately a scar made of collagen, containing a small number of fibroblasts is left.

In various embodiments, methods described herein can treat either recent or older injuries, or combinations thereof. Inflammation can be classified as either acute or chronic. Acute inflammation is the initial response of the body to harmful stimuli and is achieved by the increased movement of plasma and leukocytes (especially granulocytes) from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation and is characterized by simultaneous destruction and healing of the tissue from the inflammatory process. In various embodiments, methods can treat chronic inflammation. In various embodiments, methods can treat acute inflammation. In some embodiments, method 100 can treat a combination of acute and chronic inflammation. In some embodiments, methods described herein can treat scar material in tissue at an older injury site. In some embodiments, methods described herein can treat an abscess in tissue at an older injury site. In some embodiments, methods described herein can treat damaged tissue at an older injury site. In one outcome of inflammation and healing, fibrosis can occur. Large amounts of tissue destruction, or damage in tissues unable to regenerate, cannot be regenerated completely by the body. Fibrous scarring occurs in these areas of damage, forming a scar composed primarily of collagen. The scar will not contain any specialized structures, such as parenchymal cells, hence functional impairment may occur.

According to various embodiments, methods can include non-invasive shrinkage or removal of a fibrous scar located in a portion of tissue in the joint. Such a method can include targeting the fibrous scar in ROI 115, directing ablative ultrasound energy to the fibrous scar, ablating at least a portion of the fibrous scar, and shrinking or removing the fibrous scar. The method can also include imaging the fibrous scar. The method can also include imaging the scar after the ablating at least a portion of the fibrous scar. The method can include comparing a measurement of the scar before and after the ablating step. The method can include directing acoustical pressure or cavitation to the scar after the ablating step to further break up the sear. The method can include increasing blood perfusion to the ROI 115. The method can also include any of the steps of method 100.

In another outcome of inflammation and healing, an abscess can be formed. A cavity is formed containing pus, which is a liquid comprising dead white blood cell, and bacteria mixed with destroyed cells, According to various embodiments, methods can include non-invasive removal of an abscess located in a portion of tissue in the joint. Such a method can include targeting the abscess in ROI 115, directing ablative ultrasound energy to the abscess, ablating at least a portion of the abscess, and shrinking or removing the abscess. The method can also include imaging the abscess. The method can also include imaging the abscess after the ablating at least a portion of the abscess. The method can include comparing a measurement of the abscess before and after the ablating step. The method can include directing acoustical pressure or cavitation to the scar after the ablating step to further break up the abscess. The method can include destroying bacteria located in the abscess. The method can include increasing blood perfusion to the ROI 115. The method can include administering a medicant to the ROI 115. The method can also include any of the steps of method 100.

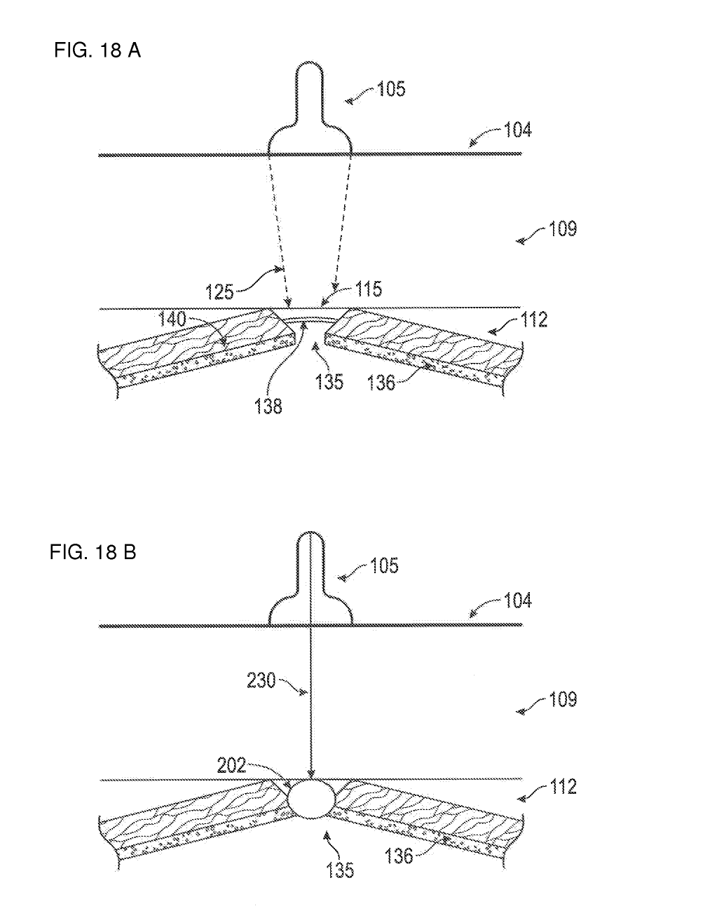

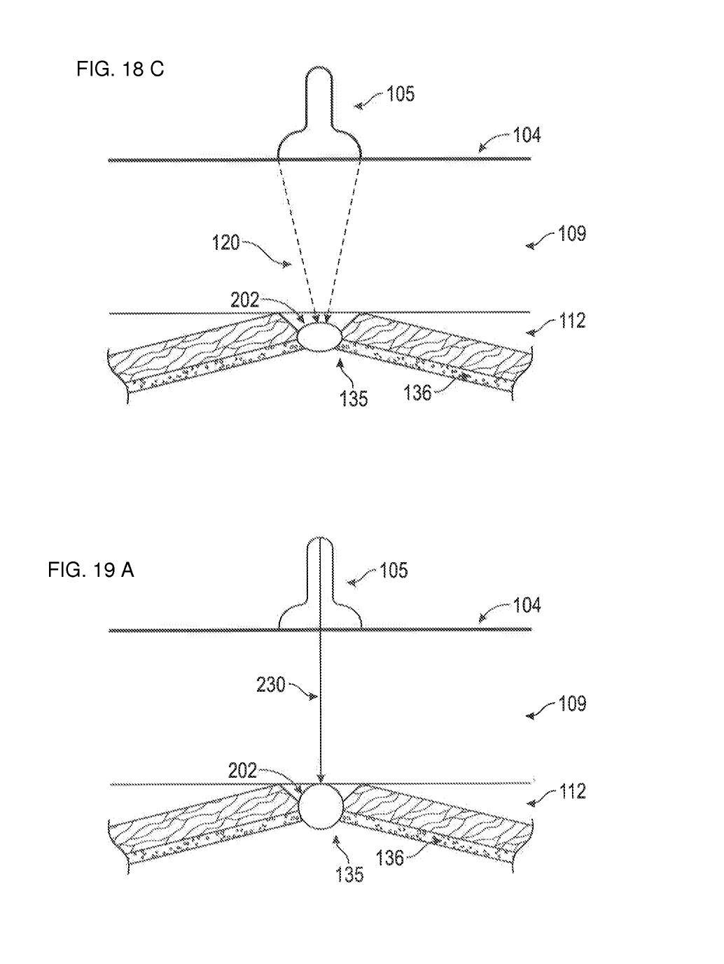

With reference to FIGS. 18A-C, method and apparatus for treating injuries to joints are illustrated. According to various embodiments, joint 135 located below surface 104. Between joint 135 and surface 104 is subcutaneous tissue 109 which can comprise muscle 112. As discussed herein, subcutaneous tissue 109 can comprise various layers such as an epidermal layer, a dermal layer, a fat layer, a SMAS layer, connective tissue, and/or muscle. Joint 135 can comprise bone 136, cartilage 140, and/or tendon 138. In various embodiments, probe 105 can be coupled to surface 104 and can emit ultrasound energy 125 into ROI 115. In various embodiments, a method can comprise imaging ROI 115 and in some embodiments, ROI 115 can comprise joint 135.

In various embodiments, needle 230 can be inserted through surface 104 and employed to direct medicant 202 to joint 135. In other embodiments, ultrasound energy can create a pressure gradient to direct medicant 202 through surface 104 to joint 135. In various embodiments, therapeutic ultrasound energy 120 is directed to joint 135. In some embodiments, therapeutic ultrasound energy 120 can ablate a portion of joint 135. The some embodiments, therapeutic ultrasound energy 120 can be focused to a portion of joint 135. In some embodiments, therapeutic ultrasound imaging 120 can create a lesion in a portion of joint 135. In some embodiments, therapeutic ultrasound energy can coagulate a portion of joint 135. In some embodiments, therapeutic ultrasound energy 120 can weld a portion of joint 135, such as for example tendon 138. In some embodiments, therapeutic ultrasound energy 120 increases blood perfusion to joint 135. In some embodiments, therapeutic ultrasound energy accelerates inflammation peaking which may stimulate healing in joint 135. In some embodiments, therapeutic ultrasound energy 120 activates medicant 202. For example, medicant 202 can be one of Etanercept, Abatacept, Adalimumah, or Infliximab, which is direct to joint 135 and therapeutic ultrasound energy 125 can be directed to the joint 135 to improve joint 135. A second medicant 202 can be PRP which is directed to joint 135 following the therapeutic ultrasound energy 125. In a further example, therapeutic ultrasound energy 125 can be directed to the joint 135 to activate the PRP and improve joint 135.

Medicant 202 can be any chemical or naturally occurring substance that has an active component. For example a medicant 202 can be, but not limited to, a pharmaceutical, a drug, a medication, a vaccine, an antibody, a nutriceutical, an herb, a vitamin, a cosmetic, an amino acid, a protein, a sugar, a recombinant material, a collagen derivative, blood, blood components, somatic cell, gene therapy, tissue, recombinant therapeutic protein, stem cells, a holistic mixture, an anti-inflammatory, or combinations thereof or mixtures thereof. Medicant 202 can also include a biologic, such as for example a recombinant DNA therapy, synthetic growth hormone, monoclonal antibodies, or receptor constructs or combinations thereof or mixtures thereof.

Medicant 202 can be administered by applying it to the skin above the ROI. Medicant 202 can be driven into subcutaneous tissue below the skin by ultrasound energy. The ultrasound energy may be provide mechanical motion, such as, vibrational, cavitation, harmonics, and/or pressure gradients, or provide a thermal gradient. A medicant 202 can be mixed in a coupling gel or can be used as a coupling gel. The medicant 202 can be administered to the circulatory system, For example, the medicant 202 can be in the blood stream and can be activated or moved to the ROI by the ultrasound energy. Medicant 202 can be administered by injection into or near the ROI. The medicant 202 can be activated by ultrasound energy.

Any naturally occurring proteins, stem cells, growth factors and the like can be used as medicant 202 in accordance to various embodiments. A medicant 202 can also include adsorbent chemicals, such as zeolites, and other hemostatic agents are used in sealing severe injuries quickly. Medicant 202 can be thrombin and/or fibrin glue, which can be used surgically to treat bleeding and to thrombose aneurysms. Medicant 202 can include Desmopressin, which can be used to improve platelet function by activating arginine vasopressin receptor 1A. Medicant 202 can include coagulation factor concentrates, which can be used to treat hemophilia, to reverse the effects of anticoagulants, and to treat bleeding in patients with impaired coagulation factor synthesis or increased consumption. Prothrombin complex concentrate, cryoprecipitate and fresh frozen plasma are commonly-used coagulation factor products. Recombinant activated human factor VII can be used in the treatment of major bleeding. Medicant 202 can include tranexamic acid and arninocaproic acid, which can inhibit fibrinolysis, and lead to a de facto reduced bleeding rate, In addition, medicant 202 can include steroids, (anabolic steroids and/or costisol steroids), for example glucocorticoid cortisol or prednisone. Medicant 202 can include can include compounds as alpha lipoic acid, DMAE, vitamin C ester, tocotrienols, and phospholipids.

Medicant 202 can be a pharmaceutical compound such as for example, cortisone, Etanercept, Abatacept, Adalimumab, or Infliximab. Medicant 202 can include platelet-rich plasma (PRP), mesenchymal stem cells, or growth factors. For example, PRP is typically a fraction of blood that has been centrifuged. The PRP is then used for stimulating healing of the injury. The PRP typically contains tbrombocytes (platelets) and cytokines (growth factors). The PRP may also contain thrombin and may contain fibenogen, which when combined can form fibrin glue. Medicant 202 can be a prothrombin complex concentrate, cryoprecipitate and fresh frozen plasma, which are commonly-used coagulation factor products. Medicant 202 can be a recombinant activated human factor VII, which can be used in the treatment of major bleeding. Medicant 202 can include tranexamic acid and aminocaproic acid, can inhibit fibrinolysis, and lead to a de facto reduced bleeding rate. In some embodiments, medicant can be Botox.

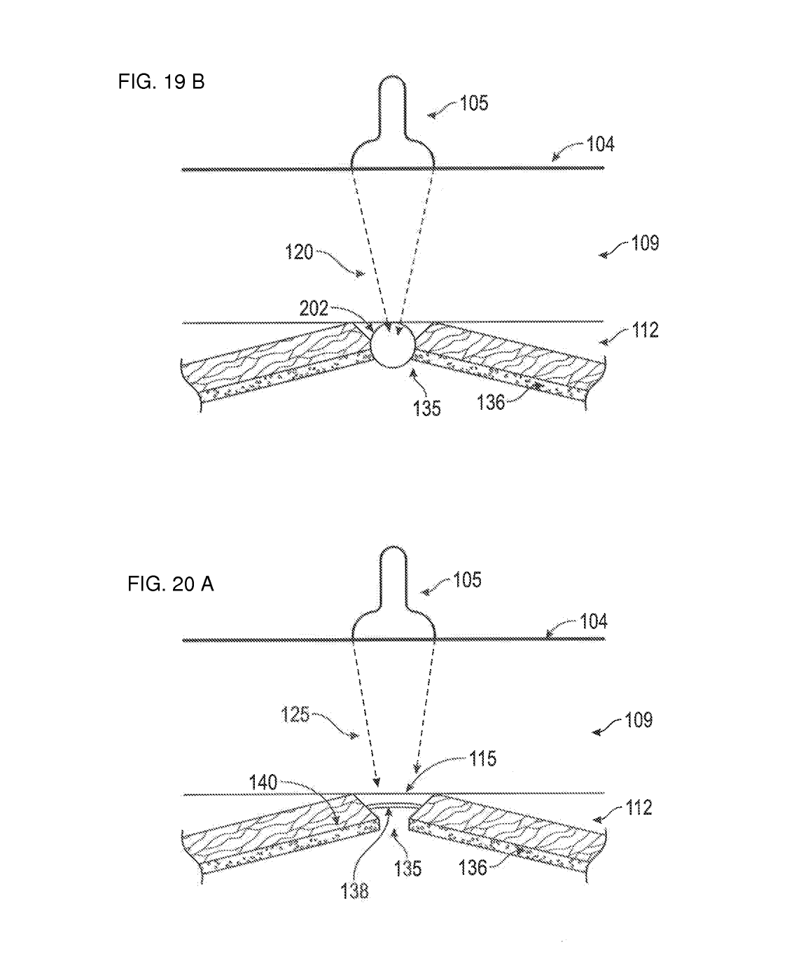

With reference to FIGS. 19A-B, method and apparatus for treating injuries to joints are illustrated. According to various embodiments, joint 135 located below surface 104. In various embodiments, needle 230 can be inserted through surface 104 and employed to direct medicant 202 to joint 135. In other embodiments, ultrasound energy can create a pressure gradient to direct medicant 202 through surface 104 to joint 135. In various embodiments, therapeutic ultrasound energy 120 is directed to joint 135. In some embodiments, therapeutic ultrasound energy 120 can ablate a portion of joint 135. The some embodiments, therapeutic ultrasound energy 120 can be focused to a portion of joint 135. In some embodiments, therapeutic ultrasound imaging 120 can create a lesion in a portion of joint 135. In some embodiments, therapeutic ultrasound energy can coagulate a portion of joint 135. In some embodiments, therapeutic ultrasound energy 120 can weld a portion of joint 135, such as for example tendon 138. In some embodiments, therapeutic ultrasound energy 120 increases blood perfusion to joint 135. In some embodiments, therapeutic ultrasound energy accelerates inflammation peaking which may stimulate healing in joint 135. In some embodiments, therapeutic ultrasound energy 120 activates medicant 202. For example, medicant 202 can be one of Etanercept, Abatacept, Adalimumab, or Infliximab, which is direct to joint 135 and therapeutic ultrasound energy 125 can be directed to the joint 135 to improve joint 135. A second medicant 202 can be PRP which is directed to joint 135 following the therapeutic ultrasound energy 125. In a further example, therapeutic ultrasound energy 125 can be directed to the joint 135 to activate the PRP and improve joint 135.

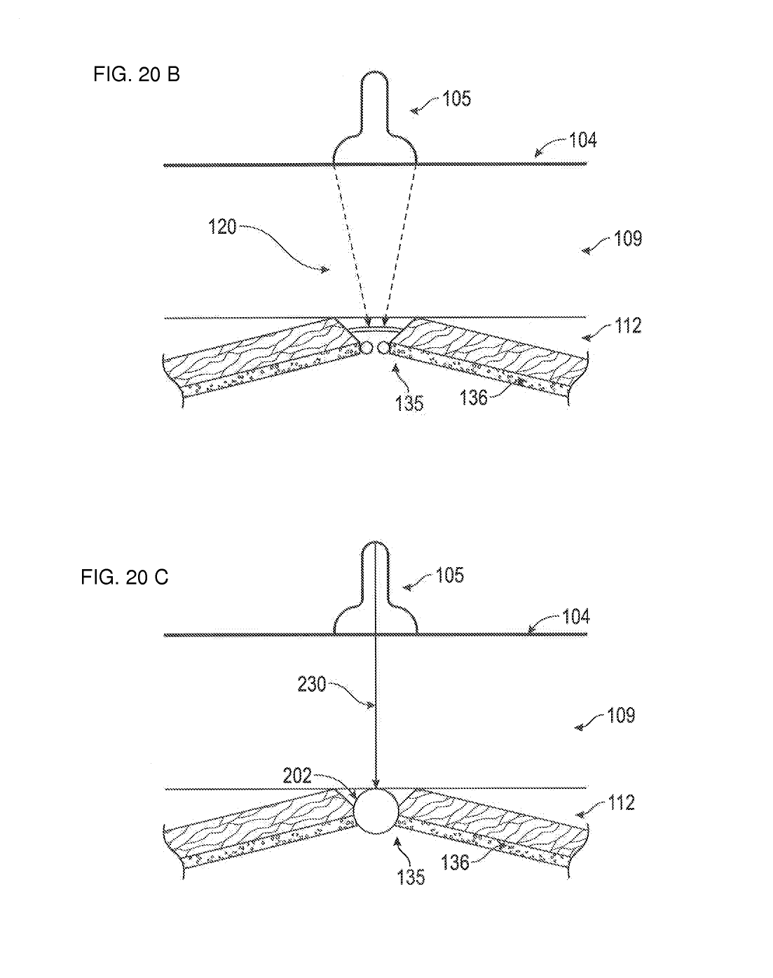

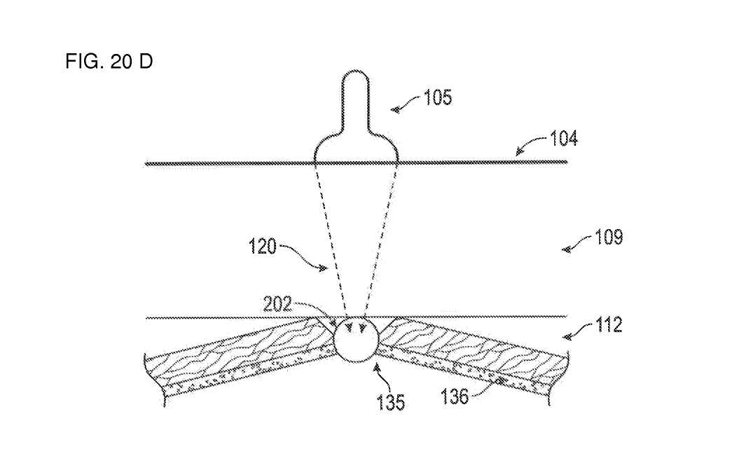

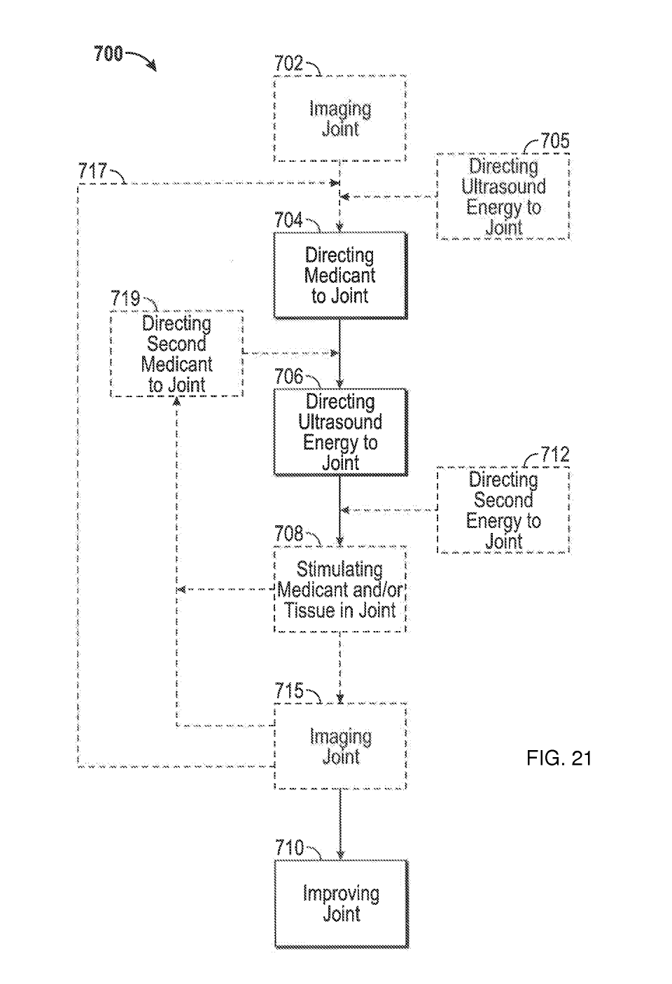

Moving to FIGS. 20A-D, method and apparatus for accelerating integration of implant into a site are illustrated. According to various embodiments, joint 135 located below surface 104. Between joint 135 and surface 104 is subcutaneous tissue 109 which can comprise muscle 112. In various embodiments, therapeutic ultrasound energy 120 is directed to joint 135. In some embodiments, therapeutic ultrasound energy 120 can ablate a portion of joint 135. In some embodiments, therapeutic ultrasound energy 120 can be focused to a portion of joint 135. In some embodiments, therapeutic ultrasound imaging 120 can create a lesion in a portion of joint 135, In some embodiments, therapeutic ultrasound energy can coagulate a portion of joint 135. In some embodiments, therapeutic ultrasound energy 120 can weld a portion of joint 135, such as for example tendon 138. In some embodiments, therapeutic ultrasound energy 120 increases blood perfusion to joint 135. In some embodiments, therapeutic ultrasound energy accelerates inflammation peaking which may stimulate healing in joint 135.

In various embodiments, needle 230 can be inserted through surface 104 and employed to direct medicant 202 to joint 135. In other embodiments, ultrasound energy can create a pressure gradient to direct medicant 202 through surface 104 to joint 135. In various embodiments, therapeutic ultrasound energy 120 is directed to joint 135. In some embodiments, therapeutic ultrasound energy 120 can ablate a portion of joint 135. The some embodiments, therapeutic ultrasound energy 120 can be focused to a portion of joint 135. In some embodiments, therapeutic ultrasound imaging 120 can create a lesion in a portion of joint 135. In some embodiments, therapeutic ultrasound energy can coagulate a portion of joint 135. In some embodiments, therapeutic ultrasound energy 120 can weld a portion of joint 135, such as for example tendon 138. In some embodiments, therapeutic ultrasound energy 120 increases blood perfusion to joint 135. In some embodiments, therapeutic ultrasound energy accelerates inflammation peaking which may stimulate healing in joint 135. In some embodiments, therapeutic ultrasound energy 120 activates medicant 202. For example, medicant 202 can be one of Etanercept, Abatacept, Adalimumab, or Infliximab, which is direct to joint 135 and therapeutic ultrasound energy 125 can be directed to the joint 135 to improve joint 135. A second medicant 202 can be PRP which is directed to joint 135 following the therapeutic ultrasound energy 125. In a further example, therapeutic ultrasound energy 125 can be directed to the joint 135 to activate the PRP and improve joint 135.

Now referring to FIG. 21, a method of treating injury in a joint is illustrated. In some embodiments, a method can optionally include imaging joint 702. In various embodiments, a method can include placing or directing a medicant 704 to joint. In some embodiments, a method can optionally include directing therapeutic ultrasound energy 705 to the site before the placing or directing a medicant 704 to joint. In various embodiments, a method can include directing therapeutic ultrasound energy 706 joint. In various embodiments, a method can include stimulating or activating 708 at least one of medicant and native tissue in the joint. In some embodiments, a method can optionally include directing a second energy 712 to the joint after include directing therapeutic ultrasound energy 706 to joint. In various embodiments, method can include improving joint 710. In some embodiments, a method can include imaging joint 715 after stimulating or activating 708 at least one of medicant and native tissue in the joint In some embodiments, the method can include placing a second medicant to joint 719 then directing therapeutic ultrasound energy 706 to joint. In some embodiments, after imaging joint 715, a decision 717 can be made to loop back and repeat certain steps of method as described herein. As will be apparent to those skilled in the art, hashed lines and hashed boxes indicate steps which are optional in method 700.

In various embodiments, method 700 can treat either recent or older injuries, or combinations thereof. Inflammation can be classified as either acute or chronic, as described herein. In various embodiments, method 700 can treat chronic inflammation. In various embodiments, method 700 can treat acute inflammation. In some embodiments, method 700 can treat a combination of acute and chronic inflammation.

In various embodiments, method 700 can include improving joint 710, which can include initiating a biological effect. A biological effect can be stimulating or increase an amount of heat shock proteins. Such a biological effect can cause white blood cells to promote healing of a portion of the subcutaneous layer in joint. A biological effect can be to restart or increase the wound healing cascade in joint. A biological effect can be increasing the blood perfusion in joint. A biological effect can be encouraging collagen growth. A biological effect may increase the liberation of cytokines and may produce reactive changes in joint. A biological effect may by peaking inflammation in joint. A biological effect may be the disruption or modification of biochemical cascades. A biological effect may be the production of new collagen. A biological effect may be a stimulation of cell growth in joint. A biological effect may be angiogenesis. A biological effect may be stimulation or activation of coagulation factors. A biological effect may a cell permeability response. A biological effect may be an enhanced delivery of medicants in joint.

In various embodiments, therapeutic ultrasound energy changes at least one of concentration and activity of inflammatory mediators (TNF-A, IL-1) as well as growth factors (TGF-B1, TGF-B3) at site. In various embodiments, therapeutic ultrasound energy accelerates inflammation peaking, which can accelerate various healing cascades.

In various embodiments, method 700 can include improving joint 710, which can include stimulating a change in at least one of concentration and activity of one or more of the following: Adrenomeduilin (AM), Autocrine motility factor, Bone morphogenetic proteins (BMPs), Brain-derived neurotrophic factor (BDNF), Epidermal growth factor (EGF), Erythropoietin (EPO), Fibroblast growth factor (FGF), Glial cell line-derived neurotrophic factor (GDNF), Granulocyte colony-stimulating factor (G-CSF), Granulocyte macrophage colony-stimulating factor (GM-CSF), Growth differentiation factor-9 (GDF9), Hepatocyte growth factor (HGF), Hepatoma-derived growth factor (HDGF), Insulin-like growth factor (IGF), Migration-stimulating factor, Myostatin (GDF-8), Nerve growth factor (NGF) and other neurotrophins, Platelet-derived growth factor (PDGF), Thrombopoietin (TP0), Transforming growth factor alpha(TGF-α.), Transforming growth factor beta(TGF-β), Tumor necrosis factor-alpha(TNF-α), Vascular endothelial growth factor (VEGF), Wnt Signaling Pathway, placental growth factor (PIGF), [(Foetal Bovine Somatotrophin)] (FBS), IL-1-Cofactor for IL-3 and IL-6, which can activate T cells, IL-2-T-cell growth factor, which can stimulate IL-1 synthesis and can activate B-cells and NK cells, IL-3, which can stimulate production of all non-lymphoid cells, IL-4-Growth factor for activating B cells, resting T cells, and mast cells, IL-5, which can induce differentiation of activated B cells and eosinophils, IL-6, which can stimulate Ig synthesis and growth factor for plasma cells, IL-7 growth factor for pre-B cells, and/or any other growth factor not listed herein, and combinations thereof.

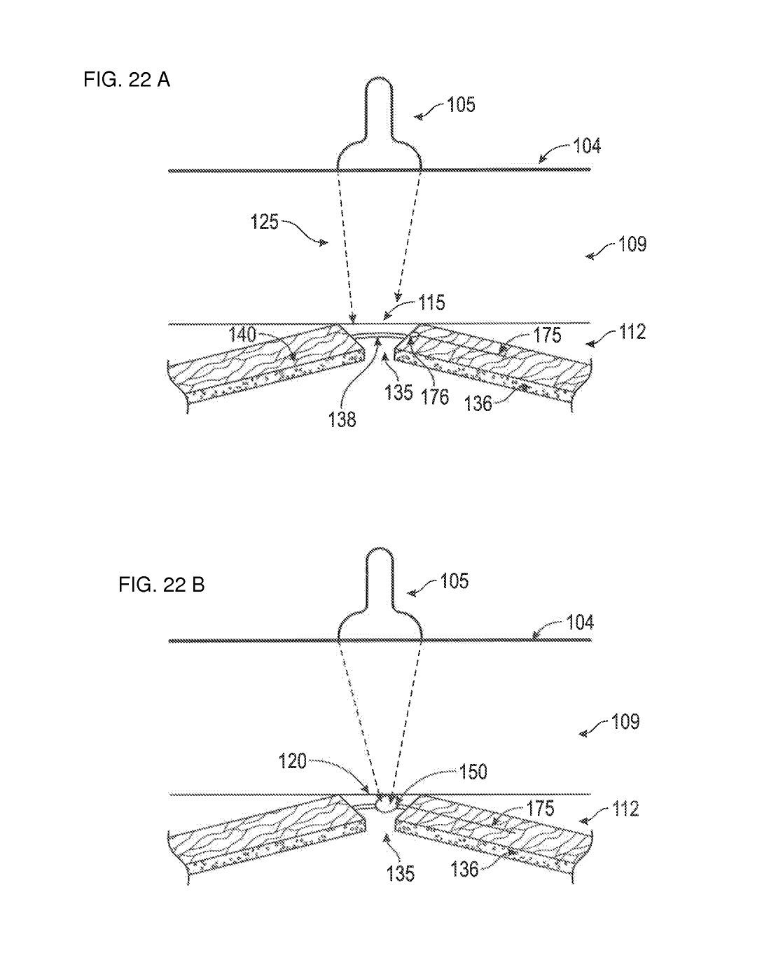

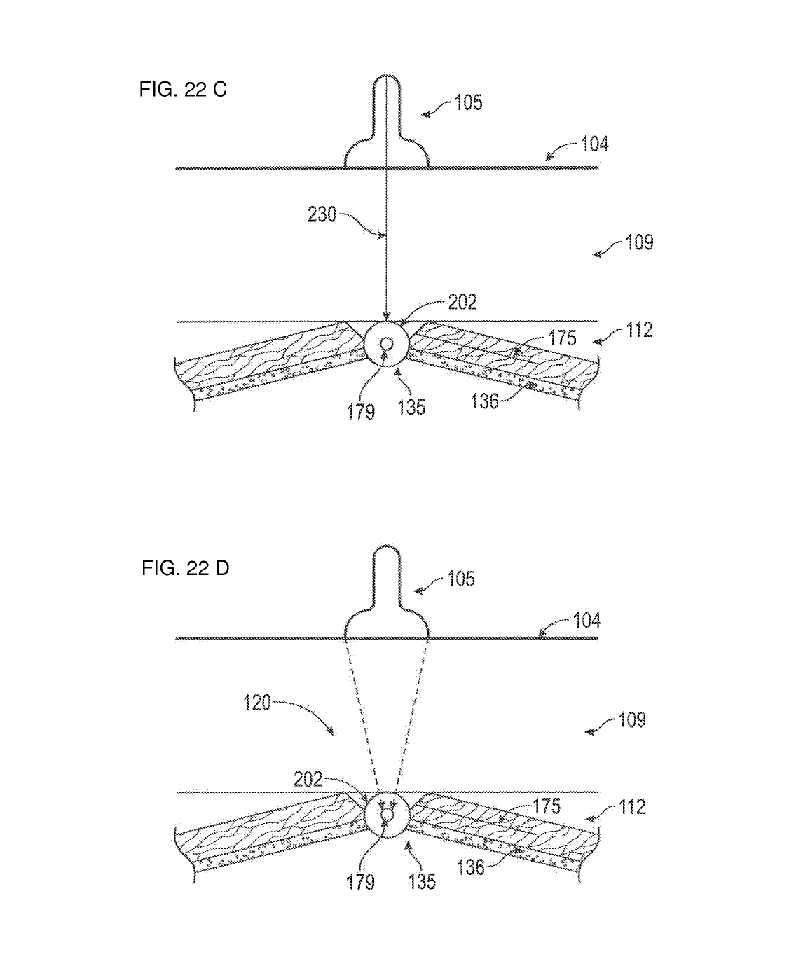

Turning to FIGS. 22 A-D, method and apparatus for permanent pain relief in joints are illustrated. According to various embodiments, joint 135 located below surface 104. Between joint 135 and surface 104 is subcutaneous tissue 109 which can comprise muscle 112. As discussed herein, subcutaneous tissue 109 can comprise various layers such as an epidermal layer, a dermal layer, a fat layer, a SMAS layer, connective tissue, and/or muscle. Joint 135 can comprise bone 136, cartilage 140, and/or tendon 138. Nerve 175 is connected to joint 135 and nerve ending 176 is part of joint 135. In some embodiments, pain in joint 135 is generated by nerve ending 176.

In various embodiments, probe 105 can be coupled to surface 104 and can emit ultrasound energy 125 into ROI 115. In various embodiments, a method can comprise imaging ROI 115 and in some embodiments, ROI 115 can comprise joint 135. In some embodiments, ROI 115 can comprise nerve ending 176. In various embodiments, therapeutic ultrasound energy 120 is directed to nerve ending 176. In some embodiments, therapeutic ultrasound energy 120 can ablate nerve ending 176. In some embodiments, therapeutic ultrasound energy 120 can be focused to a portion of nerve ending 176. In some embodiments, therapeutic ultrasound imaging 120 can create a lesion in a portion of nerve ending 176. In some embodiments, therapeutic ultrasound imaging 120 can destroy nerve ending 176.

In various embodiments, destruction of nerve ending 176 can provide permanent pain relief in joint 135. Nerve ending 176 can be a sensory nerve and typically is not a nerve that controls motor function. In some embodiments, destruction of nerve ending 176 can employ a combination of therapeutic ultrasound energy 120 and deposition of medicant 202, such as for example Botox, on nerve ending 176. In some embodiments, deposited medicant 202 can be directed to surrounding tissue 179 near nerve ending 176 to stimulate healing of the tissue.

In various embodiments, needle 230 can be inserted through surface 104 and employed to direct medicant 202 to joint 135. In other embodiments, ultrasound energy can create a pressure gradient to direct medicant 202 through surface 104 to joint 135. In various embodiments, therapeutic ultrasound energy 120 is directed to surrounding tissue 179 near nerve ending 176. In some embodiments, therapeutic ultrasound energy 120 can ablate a portion surrounding tissue 179 near nerve ending 176. In some embodiments, therapeutic ultrasound energy 120 can be focused to a portion of surrounding tissue 179 near nerve ending 176. In some embodiments, therapeutic ultrasound imaging 120 can create a lesion in a portion surrounding tissue 179 near nerve ending 176. In some embodiments, therapeutic ultrasound energy can coagulate a portion of surrounding tissue 179 near nerve ending 176. In some embodiments, therapeutic ultrasound energy 120 can weld a portion of surrounding tissue 179 near nerve ending 176. In some embodiments, therapeutic ultrasound energy 120 increases blood perfusion to surrounding tissue 179 near nerve ending 176. In some embodiments, therapeutic ultrasound energy accelerates inflammation peaking which may stimulate healing in surrounding tissue 179 near nerve ending 176. In some embodiments, therapeutic ultrasound energy 120 activates medicant 202. For example, medicant 202 can be Botox, which is direct to nerve ending 176 and therapeutic ultrasound energy 125 can be directed to the joint 135 to permanently remove pain from joint 135. A second medicant 202 can be PRP which is directed to joint 135 following the therapeutic ultrasound energy 125. In a further example, therapeutic ultrasound energy 125 can be directed to the joint 135 to activate the PRP and improve joint 135.

In various embodiments, cartilage 140 between the joints is treated with method 100 or method 700 or variations thereof. In this regard, swollen or otherwise injured cartilage 140 responsible for osteoarthritis, rheumatoid arthritis, and juvenile rheumatoid arthritis can be treated with method 100. For example, ROI 115 may be along a patient's knees to treat cartilage that serves as a cushion in a patient's knee socket Alternatively, ROI 115 can be disposed on a patient's shoulder area to treat cartilage 140 disposed on the shoulder joint. In some embodiments, therapeutic ultrasound energy 120 may not be applied at ablative levels but at levels that produce enough heat at ROI 115 to reduce swelling and the size of cartilage 140 within these joints. In various embodiments, needle 230 can be inserted through surface 104 and employed to direct medicant 202 to joint 135.

In various embodiments, cartilage between bones in the spine is treated by method 100. In an exemplary embodiment, methods described herein may be used to treat degenerative disc disease. Still further, methods described herein may be used to treat a disc in the spine. For example, methods described herein may be used to weld a tear in a disc together. In another example, methods and systems described herein may be used to perform an intervertebral disc annuloplasty, whereby a disc is heated to over 80° C. or to over 90° C. to seal a disc. In an exemplary embodiment, a method of treating a disc includes a minimally invasive procedure to couple ultrasound probe 105 to disc to be treated. In various embodiments, needle can be inserted through surface 104 and employed to direct medicant 202 to disc. In some embodiments, therapeutic ultrasound imaging 120 can destroy nerve ending 176 proximate to disc.

According to various embodiments, ultrasound probe 105 is coupled directly to cartilage, as opposed to skin surface 104, to at least one of image and treat cartilage. In some embodiments, ultrasound probe 105 can be integrated to or attached to a tool, such as, for example, an arthroscopic tool, laparoscopic tool, or an endoscopic tool that may be inserted into a patient's body with minimal invasiveness. Any steps of a minimally invasive procedure, such as arthroscopy, laparoscopy, endoscopy, and the like may be incorporated with any method described herein, including method 100 or method 700 or variations thereof.

The following patents and patent applications are incorporated by reference: US Patent Application Publication No. 20050256406, entitled “Method and System for Controlled Scanning, Imaging, and/or Therapy” published Nov. 17, 2005; US Patent Application Publication No. 20060058664, entitled “System and Method for Variable Depth Ultrasound Treatment” published Mar. 16, 2006; US Patent Application Publication No. 20060084891 entitled Method and System for Ultra-High Frequency Ultrasound Treatment” published Apr. 20, 2006; U.S. Pat. No. 7,530,958, entitled “Method and System for Combined Ultrasound Treatment” issued May 12, 2009; US Patent Application Publication No. 2008071255, entitled “Method and System for Treating Muscle, Tendon, Ligament, and Cartilage Tissue” published Mar. 20, 2008; U.S. Pat. No. 6,623,430, entitled “Method and Apparatus for Safely Delivering Medicants to a Region of Tissue Using Imaging, Therapy, and Temperature Monitoring Ultrasonic System, issued Sep. 23, 2003; U.S. Pat. No. 7,571,336, entitled “Method and System for Enhancing Safety with Medical Peripheral Device by Monitoring if Host Computer is AC Powered” issued Aug. 4, 2009; US Patent Application Publication No, 20080281255, entitled “Methods and Systems for Modulating Medicants Using Acoustic Energy” published Nov. 13, 2008 US Patent Application Publication No, 20060116671, entitled “Method and System for Controlled Thermal Injury of Human Superficial Tissue,” published Jun. 1, 2006; US Patent Application Publication No, 20060111744, entitled “Method and System for Treatment of Sweat Glands,” published May 25, 2006; US Patent Application Publication No. 20080294073, entitled “Method and System for Non-Ablative Acne Treatment and Prevention,” published Oct. 8, 2009; U.S. Pat. No. 8,133,180, entitled “Method and System for Treating Cellulite,” issued Mar. 13, 2012; U.S. Pat. No. 8,066,641, entitled “Method and System for Photoaged Tissue,” issued Nov. 29, 2011; U.S. Pat. No. 7,491,171, entitled “Method and System for Treating Acne and Sebaceous Glands,” issued Feb. 17, 2009; U.S. Pat. No. 7,615,016, entitled “Method and System for Treating Stretch Marks,” issued Nov. 10, 2009; and U.S. Pat. No. 7,530,356, entitled “Method and System for Noninvasive Mastopexy,” issued May 12, 2009.

It is believed that the disclosure set forth above encompasses at least one distinct invention with independent utility. While the invention has been disclosed in the various embodiments thereof as disclosed and illustrated herein are not to be considered in a limiting sense as numerous variations are possible. The subject matter of the inventions includes all novel and non-obvious combinations and sub combinations of the various elements, features, functions and/or properties disclosed herein.

Various embodiments and the examples described herein are one and not intended to be limiting in describing the full scope of compositions and methods of this invention. Equivalent changes, modifications and variations of various embodiments, materials, compositions and methods may be made within the scope of the present invention, with substantially similar results.

The present invention has been described in terms of one or more preferred embodiments, and it should be appreciated that many equivalents, alternatives, variations, and modifications, aside from those expressly stated, are possible and within the scope of the invention.

1. A method of non-invasive micro-fraction surgery, the method comprising:
identifying an injury location comprising damaged cartilage;
directing a conformal distribution of ultrasound energy to at least one of cartilage and surrounding subcutaneous tissue in the injury location;
ablating the at least one of cartilage and surrounding subcutaneous tissue in the injury location;
fracturing a portion of the cartilage in the injury location;
initiating re-growth of the cartilage at the injury location; and
sparing intervening tissue between a surface of skin above the injury location and the at least one of cartilage and surrounding subcutaneous tissue in the injury location.

2. The method according to claim 1, further comprising welding a portion of the cartilage at the injury location with the conformal distribution of ultrasound energy.

3. The method according to claim 1, further comprising creating a plurality of micro ablations in at least one of the cartilage and the surrounding subcutaneous tissue in the injury location.

4. The method according to claim 1, further comprising increasing blood perfusion to the injury location.

5. The method according to claim 1, wherein the surrounding subcutaneous tissue is bone.

6. The method according the claim 5, further comprising microscoring a portion of the bone with the conformal distribution of ultrasound energy to initiate re-growth of the cartilage onto the bone.

7. A method of treating an injury in a joint, the method comprising:
targeting injured fibrous soft tissue located in at least one of at and proximate to an injury location comprising a portion of a joint;
directing therapeutic ultrasound energy to the injured fibrous son tissue;
creating a conformal region of elevated temperature in the injured fibrous soft tissue; and
creating at least one thermally induced biological effect in the injured fibrous soft tissue.

8. The method according to claim 7, wherein the thermally induced biological effect is at least one of coagulation, increased perfusion, reduction of inflammation, generation of heat shock proteins, and initiation of healing cascade.

9. The method according to claim 7, further comprising targeting a capsule in the portion of the joint; and treating inflamed tissue at nr proximate to the capsule.

10. The method according to claim 7, further comprising driving a medicant into the injured soft fibrous tissue.

11. The method according to claim 10, further comprising activating the medicant with the therapeutic ultrasound energy, wherein the medicant is a steroid.

12. The method according to claim 7, further comprising peaking inflammation in the injury location and initiating a coagulation cascade in at least a portion of the joint.

13. The method according to claim 7, further comprising welding a portion of the injured fibrous soft tissue with the conformal ultrasound energy and repairing a tear in the portion of the joint.

14. The method according to claim 7, further comprising stimulating collagen growth in a portion of the joint with the conformal ultrasound energy.

15. The method according to claim 7, further comprising creating a plurality of micro lesions in a portion of a tendon of the joint; scoring a portion of the tendon; releasing strain in the tendon; and stimulating healing in the tendon.

16. The method according to claim 7, wherein the injured fibrous soft tissue is one of a muscle, a tendon, a ligament, and a capsule.
